SEQUENCE LISTING (50 sequences)

SEQ ID NO: 1 - DNA, 61 bp, Artificial sequence
  Synthetic sequence
tcaaagtgcttacagtgcaggtagtgatatgtgcatctactgcagtgaaggcacttgtagc

SEQ ID NO: 2 - DNA, 50 bp, Artificial sequence
  Synthetic sequence
ggccggatcctaatacgactcactataggtcaaagtgcttacagtgcagg

SEQ ID NO: 3 - DNA, 19 bp, Artificial sequence
  Synthetic sequence
gctacaagtgccttcactg

SEQ ID NO: 4 - DNA, 23 bp, Artificial sequence
  Synthetic sequence
caaagtgcttacagtgcaggtac

SEQ ID NO: 5 - DNA, 23 bp, Artificial sequence
  Synthetic sequence
taaggtgcatctagtgcagatag

SEQ ID NO: 6 - DNA, 23 bp, Artificial sequence
  Synthetic sequence
tgtgcaaatctatgcaaaactga

SEQ ID NO: 7 - DNA, 23 bp, Artificial sequence
  Synthetic sequence
taaagtgcttatagtgcaggtag

SEQ ID NO: 8 - DNA, 23 bp, Artificial sequence
  Synthetic sequence
tgtgcaaatccatgcaaaactga

SEQ ID NO: 9 - DNA, 22 bp, Artificial sequence
  Synthetic sequence
tattgcacttgtcccggcctgt

SEQ ID NO: 10 - DNA, 23 bp, Artificial sequence
  Synthetic sequence
acacgcaaattcgtgaagcgttc

SEQ ID NO: 11 - DNA, 20 bp, Artificial sequence
  Synthetic sequence
gaatcgagcaccagttacgc

SEQ ID NO: 12 - DNA, 27 bp, Artificial sequence
  Synthetic sequence
ggaattaattgctgttaggaggttgga

SEQ ID NO: 13 - DNA, 20 bp, Artificial sequence
  Synthetic sequence
aggtccacgtgtatgactgg

SEQ ID NO: 14 - DNA, 26 bp, Artificial sequence
  Synthetic sequence
ttatgttccctactccctacgtaagc

SEQ ID NO: 15 - DNA, 26 bp, Artificial sequence
  Synthetic sequence
agaaaagagagaaggcagaaatgctg

SEQ ID NO: 16 - DNA, 20 bp, Artificial sequence
  Synthetic sequence
gatgggcactgtccgagtag

SEQ ID NO: 17 - DNA, 19 bp, Artificial sequence
  Synthetic sequence
gcaacgtgtcatcgtgctc

SEQ ID NO: 18 - DNA, 22 bp, Artificial sequence
  Synthetic sequence
ggcacgaactgacaagacttga

SEQ ID NO: 19 - DNA, 17 bp, Artificial sequence
  Synthetic sequence
tgtggcgacgtgattaa

SEQ ID NO: 20 - DNA, 20 bp, Artificial sequence
  Synthetic sequence
gtaacccgttgaaccccatt

SEQ ID NO: 21 - DNA, 20 bp, Artificial sequence
  Synthetic sequence
ccatccaatcggtagtagcg

SEQ ID NO: 22 - DNA, 20 bp, Artificial sequence
  Synthetic sequence
gttcgacagtcagccgcatc

SEQ ID NO: 23 - DNA, 19 bp, Artificial sequence
  Synthetic sequence
ggaatttgccatgggtgga

SEQ ID NO: 24 - RNA, 31 nt, Artificial sequence
  Synthetic sequence
gugcagguagugauaugugcaucuacugcac

SEQ ID NO: 25 - DNA, 19 bp, Artificial sequence
  Synthetic sequence
taatacgactcactatagg

SEQ ID NO: 26 - DNA, 51 bp, Artificial sequence
  Synthetic sequence
taatacgactcactatagggtgcaggtagatgatatgtgcatctactgcac

SEQ ID NO: 27 - DNA, 18 bp, Artificial sequence
  Synthetic sequence
gtgcagtagatgcacata

SEQ ID NO: 28 - DNA, 51 bp, Artificial sequence
  Synthetic sequence
taatacgactcactatagggtgcaggtagtgatatgtgcatctacctgcac

SEQ ID NO: 29 - DNA, 18 bp, Artificial sequence
  Synthetic sequence
gtgcaggtagatgcacat

SEQ ID NO: 30 - RNA, 33 nt, Artificial sequence
  Synthetic sequence
gugcagguagaugauaugugcaucuaccugcac

SEQ ID NO: 31 - DNA, 50 bp, Artificial sequence; identical to SEQ ID NO: 2
  Synthetic sequence

SEQ ID NO: 32 - DNA, 19 bp, Artificial sequence
  Synthetic sequence
gctacaagtgccttcactg

SEQ ID NO: 33 - DNA, 61 bp, Artificial sequence; identical to SEQ ID NO: 1
  Synthetic sequence

SEQ ID NO: 34 - DNA, 62 bp, Artificial sequence
  Synthetic sequence
tcaaagtgcttacagtgcaggtagtgatatgtgcatctacctgcagtgaaggcacttgtagc

SEQ ID NO: 35 - DNA, 62 bp, Artificial sequence
  Synthetic sequence
tcaaagtgcttacagtgcaggtagatgatatgtgcatctactgcagtgaaggcacttgtagc

SEQ ID NO: 36 - DNA, 59 bp, Artificial sequence
  Synthetic sequence
tcaaagtgcttacagtgcaggtagatgatatgtgcatctacctgcagtgaaggcacttg

SEQ ID NO: 37 - DNA, 48 bp, Artificial sequence
  Synthetic sequence
ggccgcatggtaatacgactcactataggtaaggtgcatctagtgcag

SEQ ID NO: 38 - DNA, 18 bp, Artificial sequence
  Synthetic sequence
ccagaaggagcacttagg

SEQ ID NO: 39 - DNA, 64 bp, Artificial sequence
  Synthetic sequence
taaggtgcatctagtgcagatagtgaagtagattagcatctactgccctaagtgctccttctgg

SEQ ID NO: 40 - DNA, 65 bp, Artificial sequence
  Synthetic sequence
taaggtgcatctagtgcagatagatgaagtagattagcatctactgccctaagtgctccttctgg

SEQ ID NO: 41 - DNA, 52 bp, Artificial sequence
  Synthetic sequence
ggccggatcctaatacgactcactatagggactaaagtgcttatagtgcagg

SEQ ID NO: 42 - DNA, 22 bp, Artificial sequence
  Synthetic sequence
actttaagtgctcataatgcag

SEQ ID NO: 43 - DNA, 61 bp, Artificial sequence
  Synthetic sequence
actaaagtgcttatagtgcaggtagtgtttagttatctactgcattatgagcacttaaagt

SEQ ID NO: 44 - DNA, 51 bp, Artificial sequence
  Synthetic sequence
ggccggatcctaatacgactcactatagggttagttttgcatagttgcact

SEQ ID NO: 45 - DNA, 21 bp, Artificial sequence
  Synthetic sequence
tcagttttgcatagatttgca

SEQ ID NO: 46 - DNA, 60 bp, Artificial sequence
  Synthetic sequence
ttagttttgcatagttgcactacaagaagaatgtagttgtgcaaatctatgcaaaactga

SEQ ID NO: 47 - DNA, 49 bp, Artificial sequence
  Synthetic sequence
ggccggatcctaatacgactcactatagggttagttttgcaggtttgca

SEQ ID NO: 48 - DNA, 21 bp, Artificial sequence
  Synthetic sequence
agtcagttttgcatggatttg

SEQ ID NO: 49 - DNA, 67 bp, Artificial sequence
  Synthetic sequence
ggttagttttgcaggtttgcatccagctgtgtgatattctgctgtgcaaatccatgcaaaactgact

SEQ ID NO: 50 - DNA, 48 bp, Artificial sequence
  Synthetic sequence
ggccggatcctaatacgactcactatagggcacaggttgggatcggtt

(19) United States
(12) Patent Application Publication
(10) Pub. No.: US 2023/0149554 A1
(43) Pub. Date: May 18, 2023

(54) TARGETED DEGRADATION OF THE ONCOGENIC MICRORNA 17-92 CLUSTER BY STRUCTURE-TARGETING LIGANDS

(71) Applicant: University of Florida Research Foundation, Incorporated, Gainesville, FL (US)

(72) Inventor: Matthew D. Disney, Jupiter, FL (US)

(73) Assignee: University of Florida Research Foundation, Incorporated, Gainesville, FL (US)

(21) Appl. No.: 17/915,985

(22) PCT Filed: Mar. 24, 2021
(86) PCT No.: PCT/US2021/023976

§ 371 Date: Sep. 29, 2022

Related U.S. Application Data
(60) Provisional application No. 63/001,936, filed on Mar. 30, 2020.

(51) Int. Cl. A61K 47/64; A61K 47/22; A61K 47/54; A61P 13/08; A61P 35/00

(52) U.S. Cl. CPC A61K 47/64; A61K 47/22; A61K 47/549; A61P 13/08; A61P 35/00

(57) ABSTRACT

Embodiments of methods are disclosed for inhibiting, regulating and/or otherwise affecting or managing the pri-miR-17-92 cluster, and certain pre-miRNA's embedded in the cluster as well as the pre-miRNA's themselves as isolated forms, as members of a library, present in oncogenic and/or polycystic cell lines and/or that are present in breast cancer, prostate cancer and/or polycystic kidney disease in animals or humans, or present in any other disease in which the pri-miR-17-92 cluster and the certain pre-miRNAs within in it cause or contribute to disease. The methods utilize compounds that target the structural feature or features of the pri-miR-17-92 cluster and/or the certain pre-miRNA's. The pre-miRNA's are members of the pre-miRNA-X group which includes one or more of pre-miR-17, pre-miR-18a, pre-miR-19a, pre-miR-19b-1, pre-miR-20a, and pre-miR-92a-1 or any combination thereof. The compounds incorporate a dimeric formula of a binding moiety for the certain pre-miRNA-X's. The binding moiety can bind or complex with structural feature(s) of the certain pre-miRNA-X's without specificity for the particular nucleotide sequence of the structural features. The binding moiety nevertheless is selective for the certain structural feature(s) so that it does not bind with other pre-miRNA's not having the certain structural feature(s).

### RELATED APPLICATIONS

This application is a national stage filing under 35 U.S.C. § 371 of International Patent Application Serial No. PCT/US2021/023976, filed Mar. 24, 2021, which claims the benefit under 35 U.S.C. § 119(e) of the filing date of U.S. Provisional Application Ser. No. 63/001,936, filed Mar. 30, 2020, the entire contents of each of which are incorporated herein by reference in their entirety.

### STATEMENT OF GOVERNMENT SUPPORT

This invention was made with Government support under grant number R01 GM097455 awarded by the National Institutes of Health. The Government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Sep. 27, 2022, is named U120270107US01-SEQ-JDH and is 10,591 bytes in size.

### BACKGROUND

RNA is involved with a myriad of cellular roles beyond merely encoding and assembling proteins. The Encyclopedia of DNA Elements project and subsequent analyses showed that only 1-2% of our genome encodes for protein yet about 80% of it is transcribed into RNA (ENCODE, 2012). Although the majority of transcribed RNAs are non-coding, many non-coding RNAs are functionally involved in modulating cell activities and disease states.

The development of therapeutics that target RNA has mostly centered on using oligonucleotides. RNA is most commonly targeted with antisense oligonucleotide-based modalities (ASOs), a strategy developed in the late 1970's by Paul Zamecnik and co-workers.^{1,2} Since this landmark discovery, much activity in the area has shown that RNA biology can be affected by simple binding of the ASO or by recruiting endogenous RNase H to cleave the RNA strand in the RNA-DNA hybrid.³ RNA interference (RNAi) has also emerged as an important oligonucleotide-based approach that targets an RNA for destruction; that is triggering RNA degradation is RNAi's only mode of action.⁴ Both ASOs and RNAi have achieved success in the clinic as FDA-approved medicines.⁵ CRISPR-based strategies to target RNA are rapidly emerging and have potential to impact how diseases are treated.⁶

Each oligonucleotide-based approach recognizes RNA via sequence, and various studies have shown that unstructured regions are their ideal target sites.⁷ Approximately 50% of nucleotides in an RNA target are unstructured or non-canonically paired, leaving approximately half of its sequence unavailable for targeting purposes.⁸ Further, structured regions have been shown to regulate RNA function and processing⁹ and many directly mediate disease biology. Thus, a complement to the sequence-based recognition of oligonucleotides is structure-based recognition by using organic ligands.¹⁰ Indeed, various studies have shown that RNA can be targeted with structure-binding small molecules, compounds that can decipher biology and can be developed into preclinical candidates.¹¹⁻¹³ A strategy for the sequence-based design of structure-specific ligands, named Inforna, has been developed for RNA targets.^{14,15} This approach has been deployed successfully to rescue phenotypes associated with various diseases, to exploit known biology, to provide chemical probes to understand novel RNA biology,¹⁶ and to develop lead medicines.¹⁷⁻²⁰

It is therefore an object to develop structure specific ligands that target RNA structure modalities associated with cellular abnormalities, in particular oncogenic abnormalities. A further object is the development of ligands that target structure specific sites of RNA such as the Dicer processing sites. Yet another object is the development of ligands targeting such sites in the pri miR-17-92 cluster.

### SUMMARY OF THE INVENTION

These and other objects are achieved through ligand targeting of the primary microRNA-17-92 (pri-miR-17-92) cluster which contains six microRNAs (miRNAs) that collectively act in several disease settings. Accordingly, the invention is directed to a sequence-based design of structure-specific ligands to target a common structure in the Dicer processing sites of certain pri-miRNA's and pre-miRNA's including one or more of pre-miR-17, pre-miR-18a, pre-19a, pre-19b-1, pre-miR-20a, pre-miR-92a and/or mixtures thereof. Among these embodiments of the invention are exemplary developments directed to a series of ligands which bind certain of the miRNAs of the cluster. More specifically, the exemplary developments are directed to the targeting of one or more of at least three pre-miRNAs whether embedded within the primary cluster transcript, which in preferred embodiments are pre-miRNA-Xs selected from one or more of pre-miR-17, pre-miR-18a and pre-miR-20a and/or mixtures thereof.¹ The targeting binds the pri- and/or pre miRNA-x's through the targeting of the common structure of the Dicer processing sites of these miRNA-X's. The mature miRNA's are not targeted or bound. Thus, the targeting enables inhibition of the biogenesis of the miRNA-X's of the cluster as well as inhibiting individual pre-miRNAs. These structure-specific ligands are also imbued with substituents providing the ability to cleave the pri-miR-17-92, and/or pre-miR-17, pre-miR-18a, and pre-miR-20a and mixtures thereof, whether directly or through nuclease recruitment. ¹In this specification, the notations miR-17, miR-18a, miR-19a, miR-19b-1, miR-20a and miR-92a-1 in the context of targeting and binding by embodiments of the binding compound according to the invention mean the pre-miRNA-X's and/or their embedment within the pri-miRNA complex and not the mature miRNA's resulting from cytoplasmic Dicer ribonuclease cleavage of the pre-miRNA's to yield mature miRNA's as short (20-24 nucleotide) non-coding miRNA's.

Thus, embodiments of the invention are directed to methods for targeting the above identified pri-miRNA cluster and pre-miRNA-X's including one or more of the pri-miR-17-92 cluster and pre-miRNA-X's including one or more of pre-miR-17, pre-miR-18a, and/or pre-miR-20a and/or mixtures thereof. According to the invention, embodiments of the methods for targeting enable the compound embodiments including Compound 1D, Compound 2, Compound 5, Compound 4FL and Compound 7, the structures of which are set forth in the following paragraphs, and any combination thereof to bind with the corresponding pre-miRNA-X's as well as mixtures thereof and pri-miR-17-92. In addition, the compound embodiment, Compound 1D, constitutes an agent or instrument to enable determination of appropriate ligand binding, the most potent of which is Compound 2. Preferably, the binding is selective so that Compounds 1D, 2, 5, 4FL and 7 do not bind with pre-miRNA's or pri-miRNA's that are not one or more of the pre-miRNA-X's or pri-miR17-92.

Embodiments of the invention are also directed to methods for targeting the pri-miR-17-92 and pre-miRNA-Xs at cellular level including such cell lines as TNBC breast cancer cell line, the MDA-MD-231 breast cancer cell line, the DU-145 prostate cancer cell line, and the WT 9-12 polycystic kidney cell line. The targeting is accomplished with the compounds disclosed in the following paragraphs. The targeting enables Compound 1D, Compound 2, Compound 5, Compound 4FL and Compound 7 to bind with the pri-miR17-92, one or more of the pre-miRNA-X's, or mixtures thereof. The binding demonstrates a very low dissociation constant K_{d} and selectivity. Based on the selectivity and binding capability of these compounds, their inhibitory effects upon ordinary, non-oncogenic cells such as normal human cells and cell lines are believed to be sufficiently minimal so that such non-oncogenic cells do not succumb as a result of toxicity and/or apoptosis.

Embodiments of the invention are also directed to methods for treatment of MDA-MD-231 breast cancer cells, TNBC breast cancer cells, DU-145 prostate cancer cells, or WT 9-12 polycystic kidney cells present in a host such as a laboratory animal or present as the corresponding disease in a human. The treatment enables Compound 2, Compound 5 and/or Compound 7 and any combination thereof to bind with the pre-miRNA-Xs and/or the pri-miR-17-92 of the cells. According to the invention, the binding inhibits the oncogenic and cystic formation capabilities of the pri-miR-17-92 and/or pre-miRNA-Xs. The inhibition accordingly retards and/or inhibits invasion, apoptosis, or cyst formation correspondingly.

Embodiments of the invention directed to targeting of oncogenic cell lines with Compound 2, Compound 5 and/or Compound 7 and/or any combination thereof also enable a decrease or diminishment of the invasive characteristic of TNBC cell lines, anti-apoptotic characteristic of prostate cancer cells, and the cyst formation characteristic of cystic kidney cells. The first two aspects dampen and/or inhibit oncogenic seed cell transfer from an ongoing oncogenic cell site to a new site within a host having the oncogenic cells.

Embodiments of the invention as well target the pri-miR-17-92 cluster with compound 2, compound 5 and/or compound 7. The targeting enables Compound 2, 5 and/or 7 and/or any combination thereof to bind with the pri-miR-17-92 cluster and inhibit, retard and/or repress the oncogenic and cyst formation activity of this cluster.

Yet another embodiment of the invention is directed to methods for treatment of breast cancer, prostate cancer and/or polycystic kidney disease in humans by administration of an effective dose of Compound 2, Compound 5 and/or Compound 7 and/or any combination thereof alone or as a pharmaceutical composition in which the selected compound is combined with a pharmaceutically acceptable carrier.

Compositional embodiments of the invention are directed to dimeric moiety peptidylmimetic compounds that are capable of targeting and binding one or more of the pri-miR-17-92 or pre-miRNA-X's including one or more of the three above identified pre-miRNA's and/or their mixture embedded in the cluster. A sequence-based design known as the lead identification strategy, Inforna, enabled development of these compositional embodiments. The Inforna strategy is disclosed in Velagapudi, S. P.; Gallo, S. M.; Disney, M. D., Sequence-based design of bioactive small molecules that target precursor microRNAs. Nat. Chem. Biol. 2014, 10 (4), 291-7 and Disney, M. D.; Winkelsas, A. M.; Velagapudi, S. P.; Southern, M.; Fallahi, M.; Childs-Disney, J. L., Inforna 2.0: a platform for the sequence-based design of small molecules targeting structured RNAs. ACS Chem. Biol. 2016, 11 (6), 1720-8.

In particular, the Inforna technology enabled development of compound embodiments such as Compound 1D based upon the precursor active compound, Compound 1. For the depiction of compound 1D with subscript n, n is 0 or an integer of 1 to 7 (e.g., n=0-7).

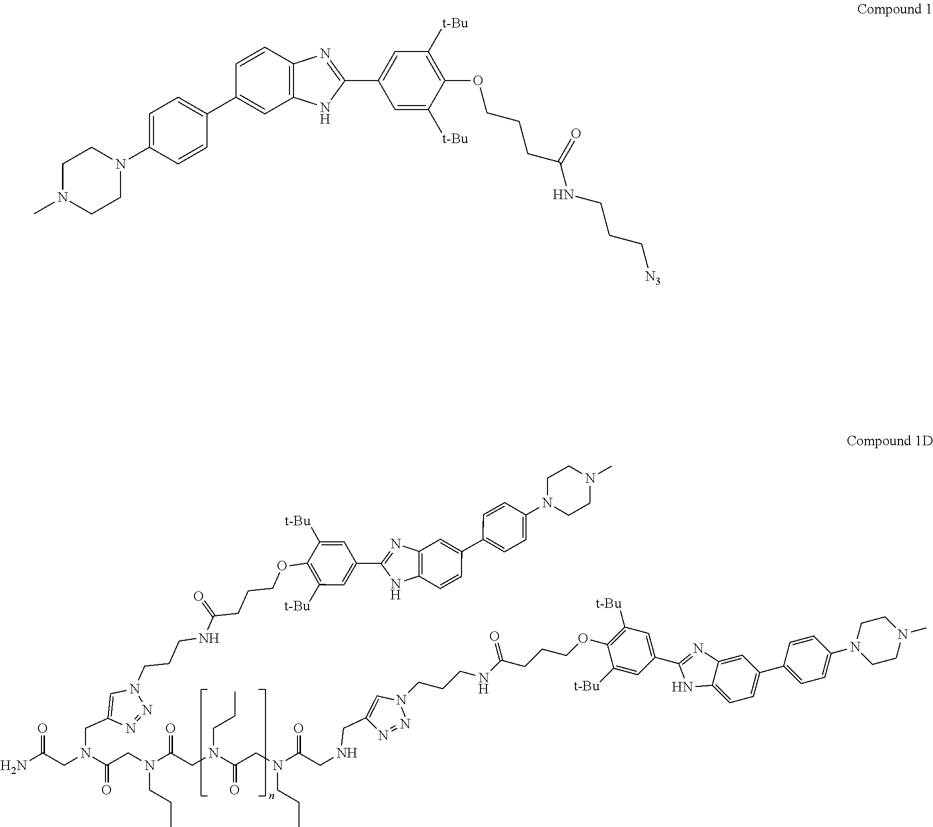

Compound 1D is a spaced dimer of Compound 1 in which the azido group of Compound 1 is coupled with an alkynyl group of the peptoid 1P by click chemistry to form a triazole ring which joins compound 1 to the peptoid 1P.

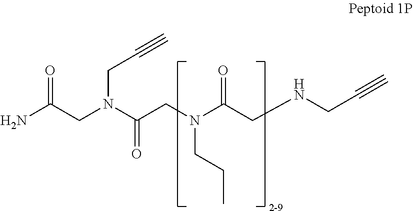

The inforna technology also enabled optimization of compound 1D to produce dimeric molecule, compound 2, that binds the Dicer processing site and an adjacent bulge, affording a 100-fold increase in potency over the investigatory compound, compound 1. Compound 2 has two forms: an amide at the tag binding site and a carboxylic acid at the tag binding site.

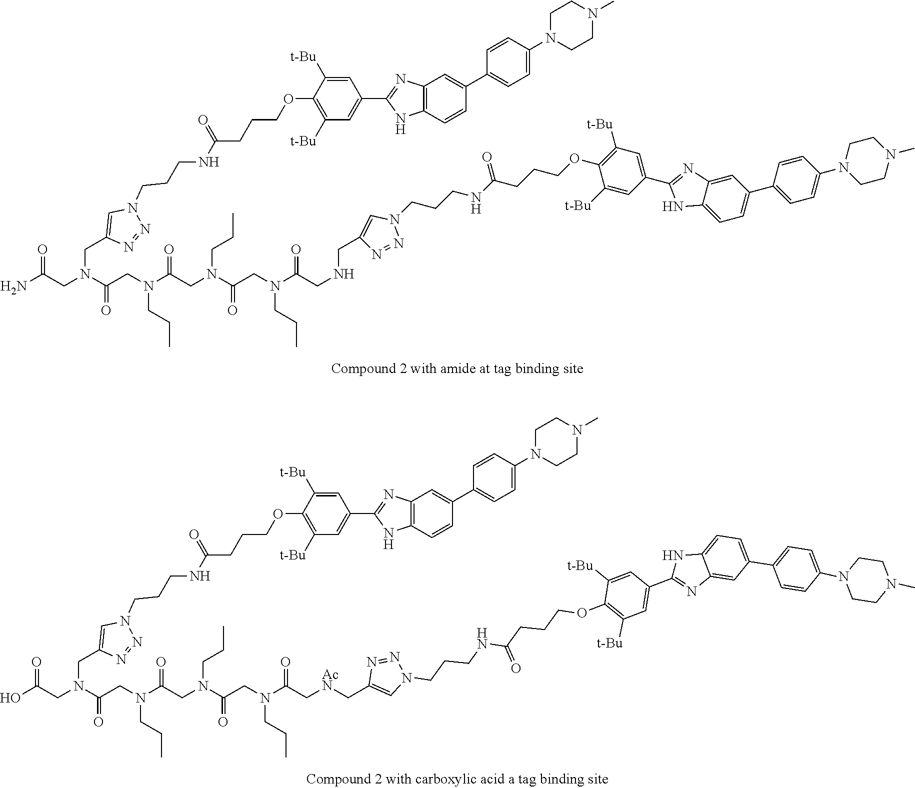

Further embodiments of the invention are directed to extension of the dimer Compound 2 mode of action from simple binding to a direct cleavage moiety by conjugation at the tag binding site to bleomycin A5 to yield Compound 5. The embodiment incorporating compound 5 imparts RNA-selective cleavage.

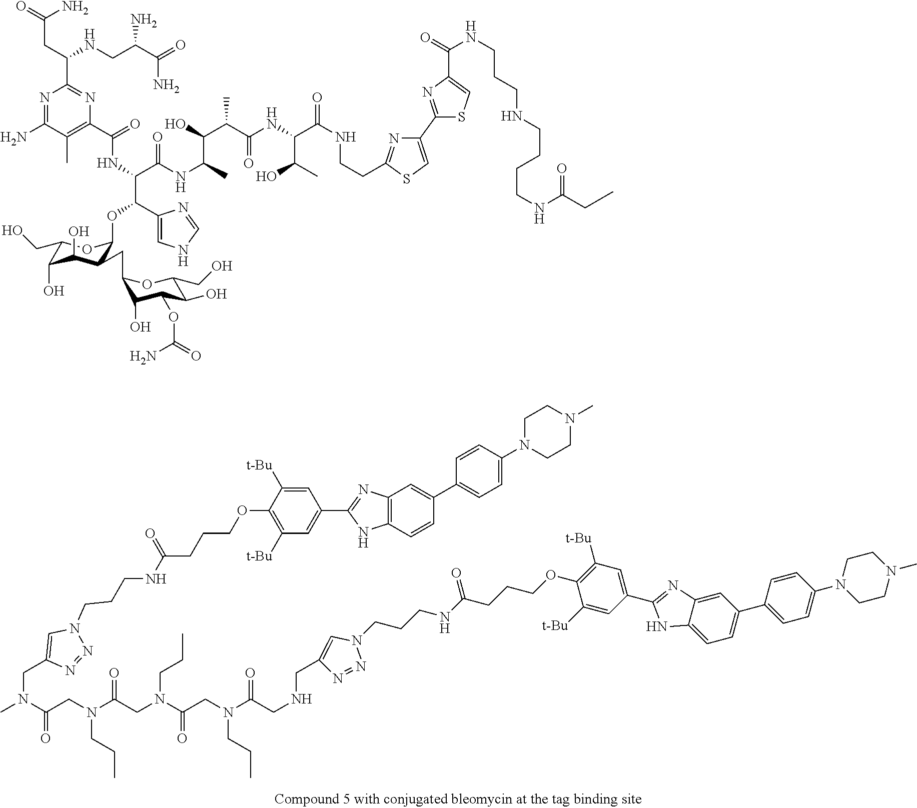

Additional embodiment extensions are directed to extension of the dimer Compound 2 by conjugation at the tag binding site to a RNase L recruiter to yield Compound 7. Compound 7 initiates indirect cleavage by recruiting an endogenous nuclease, or a ribonuclease targeting chimera (RIBOTAC).

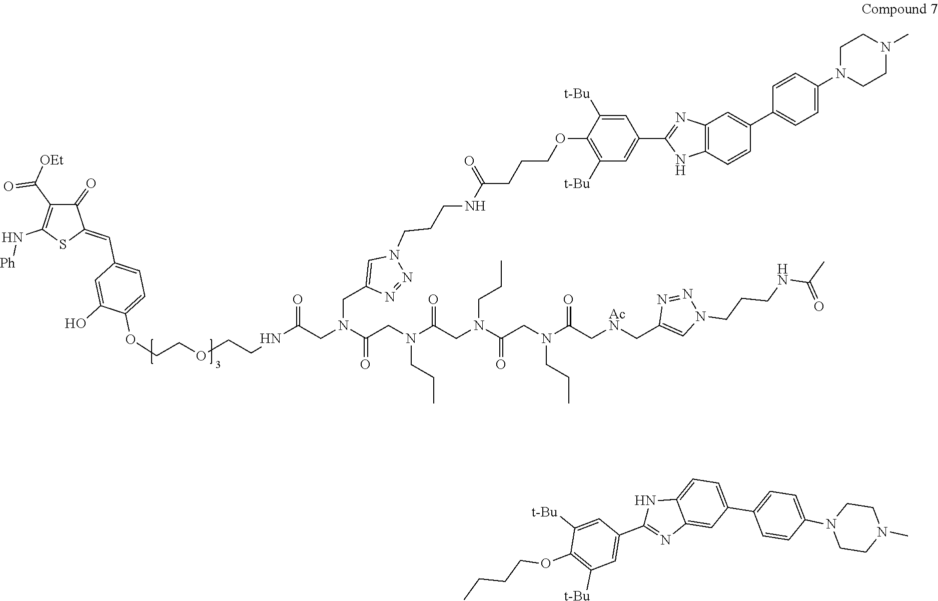

The foregoing Compounds may be formulated as pharmaceutically acceptable salts, typically using a pharmaceutically acceptable acid. The Compounds alone or as pharmaceutically acceptable salts may also be combined with a pharmaceutically acceptable carrier to produce a Pharmaceutical Composition. The Compounds and/or salts alone and/or as Pharmaceutical Compositions may be used in the foregoing methods to target as set forth above.

### BRIEF DESCRIPTION OF THE DRAWINGS

FIGS. 1A-1D show a schematic of the pri-miR-17-92 cluster and its downstream targets. FIG. 1A shows a Schematic of the polycistronic pri-miR-17-92 cluster's secondary structure. Interestingly, miR-17, miR-18a, and miR-20a share a common structure at their Dicer sites, the 1-nucleotide bulge 5′G_U/3′CUA targetable with 1. Adjacent targetable motifs are present in all three miRNAs, 5′GGU/3′C_A in miR-17 and miR-20a and 5′GAU/3′C_A in miR-18a. The SEQ ID NO:'s for the pri-miR-17-92 cluster are given in FIG. 1A and are pri-miR-17=SEQ ID NO:99; pri-miR-sh1 (sh1 for short1)=SEQ ID NO:100; pri-miR-18a=SEQ ID NO:101; pri-miR-19a=SEQ ID NO:102; pri-miR-20a=SEQ ID NO:103; pri-miR-19b-1=SEQ ID NO:104; pri-miR-sh2 (sh2 for short2)=SEQ ID NO:105; prei-miR-92a-1=SEQ ID NO:106.

FIG. 1B shows that in DU-145 prostate cancer cells, miR-18a represses STK4, which inhibits apoptosis.

FIG. 1C shows that in triple negative breast cancer cells, overexpression of miR-17 and miR-20a induces an invasive phenotype.

FIG. 1D shows that in polycystic kidney disease, miR-17, miR-19a, and miR-19b are upregulated, triggering cell proliferation and cyst formation.

FIGS. 2A and 2B show a design of a dimeric compound that binds to miR-17's Dicer site. FIG. 2A shows the chemical structures of compounds used in these studies. Compound 1 is the parent monomer targeting each motif, 2 is the dimer with three propylamine spacing modules in the peptoid backbone, allowing specific binding to each miRNA's Dicer site; 3 is a Chemical Cross-Linking and Isolation by Pull-down (Chem-CLIP) probe that contains a diazirine reactive module and click handle for conjugating biotin, enabling pull-down for assessing target engagement; 4 is the Chem-CLIP control probe that lacks the RNA-binding modules to assess non-specific reaction of the diazirine.

FIG. 2B shows the secondary structures of the model RNAs used in binding studies and the corresponding affinities for 2. Note that “miR-17 Dicer site” and “miR-18a Dicer site” are comprised of sequences native to the corresponding RNA. Other RNAs contain mutations that convert the bulges to base pairs. The SEQ ID NO:'s for these Dicer sites, Bulges and Control are: miR-17 Dicer Site=SEQ ID NO:107; miR-17 G-Bulge=SEQ ID NO:108; miR-17 U-Bulge=SEQ ID NO:109; miR-17 Base Pair Control=SEQ ID NO:110; miR-18a Dicer Site=SEQ ID NO:111

FIGS. 3A-3E show the bioactivity of dimeric binder 2 in MDA-MB-231 triple negative breast cancer cells.

FIG. 3A) Effect of dimeric binder 2 on the levels of mature miRNAs derived from the miR-17-92 cluster in MDA-MB-231 cells, as determined by RT-qPCR.

FIG. 3B shows the effect of LNAs on miR-17 and miR-20a levels as determined by RT-qPCR. FIG. 3C shows the effect of 2 on pri-miR-17-92 levels shows a 50% increase in pri levels.

FIG. 3D shows the effect of 2 on pri-miR-17-92, pre-miR-17, and pre-miR-20a levels, as determined by RT-qPCR.

FIG. 3E shows the effect of 2 on the invasive properties of MDA-MB-231 cells caused by aberrant expression of miR-17 and miR-20a. Representative images of invasion assays from 2-treated and untreated MDA-MB-231 cells. Errors are reported as S.E.M. *, p<0.05; **, p<0.01; ***, p<0.001, as determined by a Student t test.

FIGS. 4A-4E show the bioactivity of dimer binder 2 in DU-145 prostate cancer cells.

FIG. 4A shows the effect of 2 on the levels of mature miRNAs derived from the miR-17-92 cluster in DU-145 cells, as determined by RT-qPCR.

FIG. 4B shows that RT-qPCR of an LNA targeting miR-18a shows a modest effect on miR-18a levels.

FIG. 4C shows the effect of 2 on pri-miR-17-92 levels shows a 50% increase in pri levels.

FIG. 4D shows the effect of 2 on pre-miR-18a levels, as determined by RT-qPCR.

FIG. 4E shows the effect of 2 (500 nM) on phenotype (apoptosis), as measured by Caspase 3/7 activity. Errors are reported as S.E.M. *, p<0.05; **, p<0.01; ***, p<0.001, as determined by a Student t test.

FIGS. 5A-5D show a study of target engagement via Chemical Cross-Linking and Isolation by Pull-down (Chem-CLIP) and Competitive-Chem-CLIP (C-Chem-CLIP) in DU-145 cells.

FIG. 5A shows a schematic of the Chem-CLIP target engagement methodology.

FIG. 5B shows enrichment of the pri-miR17-92 transcript by 3 in Chem-CLIP studies, as compared to the lysate prior to pull-down.

FIG. 5C shows pull-down of pre-miR-18a by 3, as compared to the lysate prior to pull-down.

FIG. 5D shows C-Chem-CLIP studies in which DU-145 cells were co-treated with 3 and increasing concentrations of 2, which dose-dependently reduces the amount of pri-miR-17-92 pulled down. Errors are reported as S.E.M. *, p<0.05; **, p<0.01; ***, p<0.001, as determined by a Student t test.

FIGS. 6A-6B show a design of a small molecule to directly cleave pri-miR-17-92.

FIG. 6A shows a schematic of targeted degradation of the cluster with a cleaving compound.

FIG. 6B shows structures of the 2-bleomycin A5 conjugate, 5, and control compound 6 that lacks the RNA-binding modules.

FIGS. 7A-7G show the bioactivity of dimer-bleomycin conjugate 5 in MDA-MB-231 triple negative breast cancer cells.

FIG. 7A shows effect of cleaving compound 5 and negative control compound 6 on pri-miR-17-92 levels in MDA-MB-231 cells, as determined by RT-qPCR.

FIG. 7B shows a competition experiment between dimer binder 2 and 5 and their effect of pri-miR-17-92 levels, as determined by RT-qPCR.

FIG. 7C shows the effect of 5 on pre-miR-17 levels, as determined by RT-qPCR.

FIG. 7D shows the effect of 5 on the levels of mature miRNAs derived from the miR-17-92 cluster, as determined by RT-qPCR.

FIG. 7E shows the effect of 5 on Zbtb4 mRNA levels, a direct target of miR-17, as determined by RT-qPCR.

FIG. 7F shows the effect of 5 on ZBTB4 expression in MDA-MB-231 cells.

FIG. 7G shows the effect of 5 on the invasive characteristics of MDA-MB-231 cells, due to repression of ZBTB4. Errors are reported as S.E.M. *, p<0.05; **, p<0.01; *** p<0.001, as determined by a Student t test.

FIGS. 8A-8G show the bioactivity of the dimer-bleomycin conjugate 5 in DU-145 prostate cancer cells.

FIG. 8A shows the effect of cleaving compound 5 and negative control compound 6 on pri-miR-17-92 levels in DU-145 cells, as determined by RT-qPCR.

FIG. 8B shows a competition experiment between dimer binder 2 and 5 and their effect of pri-miR-17-92 levels, as determined by RT-qPCR.

FIG. 8C shows the effect of 5 on pre-miR-17 levels, as determined by RT-qPCR.

FIG. 8D shows the effect of 5 on the levels of mature miRNAs derived from the miR-17-92 cluster, as determined by RT-qPCR.

FIG. 8E shows the effect of 5 on Stk4 mRNA levels, a direct target of miR-18a, as determined by RT-qPCR.

FIG. 8F shows the effect of 5 on Caspase 3/7 activity, an indicator of apoptosis which is impeded in DU-145 cells due to repression of STK4.

FIG. 8G shows profiling of 373 miRNAs in DU-145 cells shows that only mature miRNAs derived from the 17-92 cluster are significantly affected, with miR-17 and -18a being the most significantly affected. Note that profiling was completed after a 6 h treatment period to minimize downstream effects as the compound induces apoptosis. All other data were collected after at 24 h treatment period. Errors are reported as S.E.M. *, p<0.05; **, p<0.01; ***, p<0.001, as determined by a Student t test.

FIGS. 9A-9F show bioactivity of RIBOTAC 7 in MDA-MB-231 TNBC and DU-145 prostate cancer cells.

FIG. 9A shows the structure of RIBOTAC 7, generated by coupling dimer binder 2 with a small molecule that recruits RNase L discovered previously.⁵⁷

FIG. 9B shows Cellular permeability of 2 (dimer binder), 5 (dimer-bleomycin conjugate) and RIBOTAC 7 at 5 μM.

FIG. 9C shows the effect of 7 on the levels of mature miRNAs from the 17-92 cluster in MDA-MB-231 TNBC cells, as determined by RT-qPCR.

FIG. 9D shows the effect of 7 on pre-miR-17, -18a, and -20a levels in MDA-MB-231 and DU-145 cells, as determined by RT-qPCR.

FIG. 9E shows the effect of 7 on pri-miR-17-92 in MDA-MB-231 TNBC and DU-145 prostate cancer cells, as determined by RT-qPCR.

FIG. 9F shows the effect of 7 on the levels of mature miRNAs from the 17-92 cluster in DU-145 prostate cancer cells, as determined by RT-qPCR. *, p<0.05, **, p<0.01, ***, p<0.001 by a Student t test. All errors are reported as S.E.M.

FIG. 10 shows a luciferase screen of dimer library for de-repression of PPARα. Screening a PPAR-α luciferase reporter for de-repression in MDA-MB-231 TNBC cells identifies a spacer length of n=3 (2) as the most optimal. Errors reported as S.E.M. ***, p<0.001, as determined by a Student t-test.

FIGS. 11A-11D show the results of in vitro Dicer inhibition of pre-miR-17 and mutants with 2.

FIG. 11A shows inhibition of in vitro Dicer processing of wild type pre-miR-17 by 2; Pre-miR-17=SEQ ID NO:112.

FIG. 11B shows inhibition of in vitro Dicer processing of pre-miR-17-G21 mutant by 2; Pre-miR-17G21 Mutant=SEQ ID NO:113.

FIG. 11C shows inhibition of in vitro Dicer processing of pre-miR-17-U37 mutant by 2; Pre-miR-17-U37 Mutant=SEQ ID NO:114.

FIG. 11D shows inhibition of in vitro Dicer processing of pre-miR-17-G21/U37 mutant by 2; Pre-miR-17-G21/U37 Mutant=SEQ ID NO:115. Errors reported as S.E.M. *, p<0.05; **, p<0.01, as determined by a Student t-test.

FIGS. 12A-12C show the results of in vitro Dicer inhibition of pre-miR-18a, and pre-miR-20a with 2.

FIG. 12A shows inhibition of in vitro Dicer processing of wild type pre-miR-18a by 2; Pre-miR-17-G21/U37 Mutant=SEQ ID NO:115.

FIG. 12B shows inhibition of in vitro Dicer processing of pre-miR-18a-U43 mutant by 2; Pre-miR-18-U43 Mutant=SEQ ID NO: 117.

FIG. 12C shows the results of inhibition of in vitro Dicer processing of wild type pre-miR-20a by 2; Pre-MiR-20a=SEQ ID NO:118. Errors reported as S.E.M. *, p<0.05; **, p<0.01, as determined by a Student t-test.

FIGS. 13A-13C show in vitro Dicer inhibition of pre-miR-19a, -19b, and 92a-1 with 2.

FIG. 13A shows inhibition of in vitro Dicer processing of wild type pre-miR-18a by 2; Pre-miR-19a=SEQ ID NO:119.

FIG. 13B shows inhibition of in vitro Dicer processing of pre-miR-18a-U43 mutant by 2; Pre-miR-19b-1=SEQ ID NO:120.

FIG. 13C shows inhibition of in vitro Dicer processing of wild type pre-miR-20a by 2; Pre-miR-92a-1=SEQ ID NO: 121. Errors reported as S.E.M. *, p<0.05; **, p<0.01, as determined by a Student t-test.

FIG. 14 shows representative microscopic images of the cellular uptake and localization of 2, 5, and 7 in DU145 cells. The images also show that all three compounds reside primarily in the cytoplasm with 5 also localizing to the perinuclear region (white arrows).

FIGS. 15A-15E show depression of \: ZBTB4 mRNA and rescue of an invasive phenotype Invasion in MDA-MB-231 cells by 2.

FIG. 15A shows absolute quantification of mature, pre- and pri-miRs in the cluster corroborates our findings by relative qPCR analysis.

FIG. 15B shows Zbtb4 mRNA levels in MDA-MB-231 upon treatment with 2, as determined by RT-qPCR.

FIG. 15C shows effect of 2 on ZBTB4 protein levels, as determined by Western blotting.

FIG. 15D shows invasion of MDA-MB-231 cells upon treatment with a scrambled oligonucleotide control and LNA-17 (100 nM).

FIG. 15E shows the effect of 2 treatment on the levels of other miRNAs predicted by TargetScan to modulate Zbtb4, as determined by RT-qPCR. Errors reported as S.E.M. *, p<0.05; **, p<0.01, as determined by a Student t test.

FIGS. 16A and 16B show the activity of 2 on the expression of miRs in miR-17-92 cluster in WT 9-12 cells.

FIG. 16A shows levels of mature miRNAs in the 17-92 cluster in WT-9-12 upon treatment of 2, as determined by RT-qPCR.

FIG. 16B is a western blot of PPARα which shows de-repression of protein upon treatment with compound 2 by ˜2.5-fold. Errors reported as S.E.M. *, p<0.05, as determined by a Student t test.

FIGS. 17A-17E show the effect of 2 on levels of miR-18a's direct target, STK4 mRNA and induction of Caspase 3/7 activity in DU-145 cells.

FIG. 17A shows absolute quantification of mature, pre- and pri-miRNAs in the cluster, as determined by RT-qPCR.

FIG. 17B shows the effect of 2 on Stk4 mRNA levels, as determined by RT-qPCR.

FIG. 17C shows the effect of 2 on STK4 protein levels, a direct target of miR-18a.

FIG. 17D shows the effect of a pool of an antago miR directed at miR-18a (LNA-18a) and a scrambled oligonucleotide control on Caspase 3/7 activity.

FIG. 17E shows effect of overexpressing the miR-17-92 cluster or knocking out Stk4 mRNA with an shRNA on 2's ability to induce Caspase 3/7 activity. Errors reported as S.E.M. *, p<0.05, **, p<0.01, ***, p<0.001, as determined by a Student t test.

FIGS. 18A-18C show the results of in vitro cleavage of pre-miR-17, mutant pre-miR-17-BP, and DNA by 5 or 6.

FIG. 18A shows results for in vitro cleavage of pre-miR-17 by 5 and 6 and an Iron (II) only control; depicted miR sequence=SEQ ID NO: 122. Compound 5 cleaves pre-miR-17 (left gel) near the Dicer site while 6 has no clear cleavage pattern (middle gel). Iron (II) has no effect (right gel).

FIG. 18B shows results for in vitro cleavage of mutant pre-miR-17 by 5 or 6 with no effect of either compound as the binding motifs are absent; depicted miR sequence=SEQ ID NO: 123.

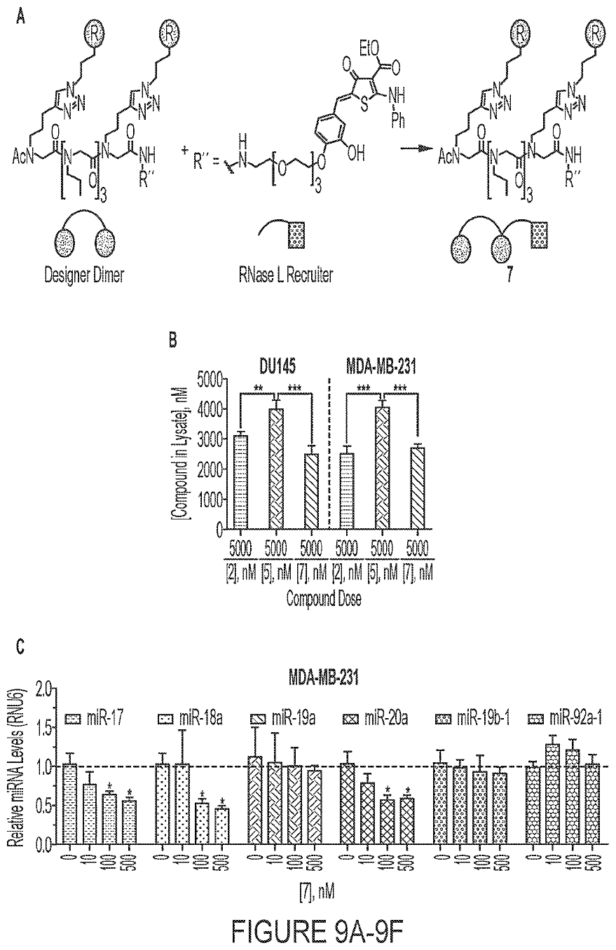

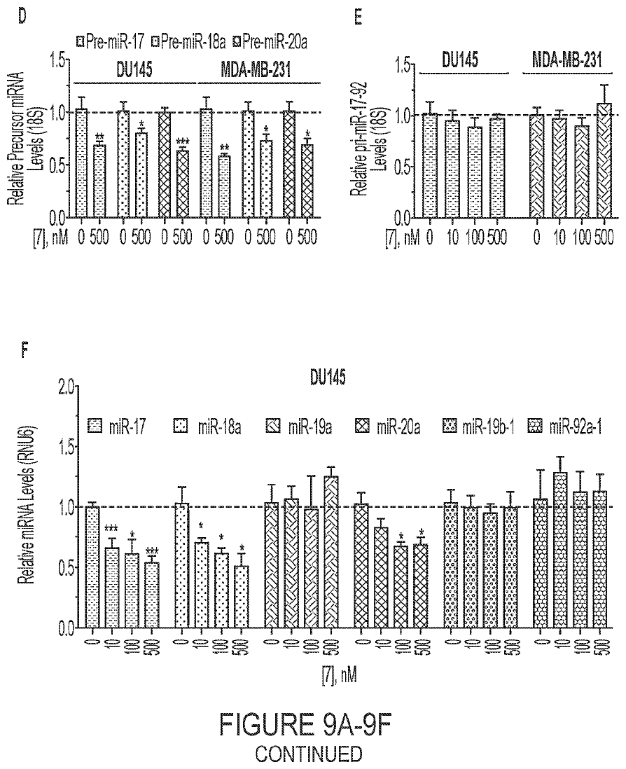

FIG. 9C shows results of cleavage of plasmid DNA by 5, 6, or bleomycin A5 in vitro. Errors reported as S.E.M. *, p<0.05, as determined by a Student t-test.

FIGS. 19A-19H show effects of 5 and 6 in MDA-MB-231 TNBC cells.

FIG. 19A shows levels of mature miRNAs from the 17-92 cluster upon treatment with 6 (lacks RNA-binding modules), as determined by RT-qPCR.

FIG. 19B shows absolute quantification of mature, pre- and pri-miRNAs in the cluster, as determined by RT-qPCR.

FIGS. 19C-19D show overexpression of the miR-17-92 cluster in MDA-MB-231 cells ablated 5's knockdown of pri-miR-17-92 and de-repression of Zbtb4 mRNA levels.

FIG. 19E shows pri-miR-17-92 levels in MDA-MB-231 cells overexpressing a shZBTB4 show no effect on 5's cleavage of pri-miR-17-92.

FIG. 19F shows effect of 5 on Zbtb4 mRNA levels in MDA-MB-231 cells expressing shZBTB4, as determined by RT-qPCR.

FIG. 19G shows effect of 5 on the invasive properties of MDA-MB-231 cells that express shZBTB4 cells.

FIG. 19H shows effect of 5 on miRNAs that share bulges bound by 1 (RNA isoforms) and miR-21. Errors reported at S.E.M *, p<0.05, **, p<0.01, ***, p<0.001 by a Student t test.

FIGS. 20A-20K show effects of compound 5 and 6 in DU-145 cells.

FIG. 20A shows levels of mature miRNAs from the 17-92 cluster upon treatment with 6 (lacks RNA-binding modules), as determined by RT-qPCR. The reduction in miR-19a levels is likely due to its stretches of AU pairs, known to be cleaved preferentially by bleomycin A5.1

FIG. 20B shows the absolute quantification of mature, pre- and pri-miRNAs in the cluster, as determined by RT-qPCR.

FIG. 20C shows a western blot of STK4 protein levels in DU145 cells treated with 5 and 6.

FIG. 20D shows the Effect of 6 on Caspase 3/7 activity DU145 cells.

FIGS. 20E-F show the effect of 5 on pri-miR-17-92 and Stk4 mRNA levels in DU145 cells overexpressing the miR-17-92 cluster, as determined by RT-qPCR.

FIG. 20G shows the effect of 5 on Caspase 3/7 activity in DU145 cells overexpressing the cluster.

FIGS. 20H-I shows the effect of 5 on pri-miR-17-92 and Stk4 mRNA levels in DU145 cells expressing a shRNA targeting Stk4 mRNA.

FIG. 20J shows the effect of 5 on Caspase 3/7 activity in DU145 cells expressing a shRNA targeting Stk4 mRNA.

FIG. 20K shows the eEffect of 5 on miRNAs that share bulges bound by 1 (RNA isoforms) and miR-21. Errors reported at S.E.M. *=p<0.05, **=p<0.01, ***=p<0.001 by a Student t test.

FIGS. 21A-21E show the global protein expression changes in DU-145 cells treated with 5.

FIG. 21A shows a volcano plot of the global proteome in DU-145 cells treated with 5 vs. vehicle, as determined by LC-MS/MS analysis. Data are represented as log 2 fold change; dotted lines represent a false discovery rate of 1% and an S0 of 0.1 [where S0 is the minimum fold change required to be considered for significance], collectively an adjusted p-value of 0.01. Colored dots represent proteins (n=9) significantly changed in response to treatment with 5.

FIG. 21B shows a comparison of fold-change in protein levels, as determined by proteomics analysis, as a function of fold-change in the encoding mRNAs, as determined by RT-qPCR. Of these, only VPS28, PD-L1, RUSC1, and TTI2 showed significant changes in their mRNA that correlated with the observed change in protein expression levels. Notably, programmed cell-death ligand 1 (PD-L1 or CDC274) is upregulated, which is a known target of miR-17 and miR-20a. PD-L1 (CD274) is a known target of miR-17; thus, its upregulation is expected (˜40%).

FIG. 21C shows that treatment with 5 has no significant increase in PD-L1 surface expression as measured by FACS.

FIGS. 21D and 21E show RT-qPCR (D) and FACS (E) analyses of DU-145 cells that overexpress PD-L1. The protein PD-L1 binds to its cognate receptor programmed cell death 1 (PD-1) to control T-cell activation. Cancer cells have increased surface levels of PD-L1 to evade T-cell mediated immune responses.²⁻³ However, this marker is challenging to target with ADC's due to either insufficient surface enhancement and its expression on other tissues.^{4,5} Increasing surface levels of PD-L1 may be a viable strategy to make this marker amenable for ADC mediated therapies. FACS analysis of DU-145 cells treated with 5, showed no significant increase in PD-L1 surface levels. We next studied how much of an increase in PD-L1 mRNA is needed to change surface levels significantly. An increase of 10-fold of PD-L1 mRNA is required to cause a 50% increase in cell surface expression. This suggests that 5, while it can de-repress PD-L1, cannot achieve a high enough increase in gene expression to alter surface levels. These do however support that 5 is indeed engaging the miR-17-92 cluster since PD-L1 is a downstream target of miR-17 and miR-20a. Errors reported as S.E.M.

FIGS. 22A and 22B show absolute quantification of Mature, precursor and primary miR-17-92 cluster in DU145 and MDA-MB-231 for RIBOTAC (7).

FIG. 22A shows the absolute quantification of mature, pre- and pri-miR-17-92 shows similar effects by 7 in MDA-MB-231 cells corroborating the relative quantification data observed previously.

FIG. 22B shows the absolute quantification of mature, pre- and pri-miR-17-92 shows similar effects by 7 in DU-145 cells corroborating the relative quantification data observed previously. All errors reported as S.E.M *, p<0.05; **, p<0.01; ***, p<0.001 by a Students T-test.

### DEFINITIONS

Unless defined otherwise, all technical and scientific terms used herein have the same meaning as commonly understood by a person of ordinary skill in the art.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

The term “may” in the context of this application means “is permitted to” or “is able to” and is a synonym for the term “can.” The term “may” as used herein does not mean possibility or chance.

The term “and/or” in the context of this application means either one alone as well as both together, for example a substance including A and/or B means a substance including A alone, a substance including B alone and a substance including A and B together. Any one of the three choices standing alone may be made as well as any combination such as A alone as well as A and B together or B alone as well as A and B together or A alone, B alone and A and B together (e.g., all three choices).

It is also to be understood that as used herein and in the appended claims, the singular forms “a,” “an,” and “the” include plural reference unless the context clearly dictates otherwise, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and the right is reserved to revise the application or claims to refer specifically to any individual member or any subgroup of members of the Markush group.

The expression “effective amount”, when used to describe therapy to an individual suffering from a disorder, refers to the amount of a drug, pharmaceutical agent or compound of the invention that will elicit the biological or medical response of a cell, tissue, system, animal or human that is being sought, for instance, by a researcher or clinician. Such responses include but are not limited to amelioration, inhibition or other action on a disorder, malcondition, disease, infection or other issue with or in the individual's tissues wherein the disorder, malcondition, disease and the like is active, wherein such inhibition or other action occurs to an extent sufficient to produce a beneficial therapeutic effect. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder.

Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

Phrases such as “under conditions suitable to provide” or “under conditions sufficient to yield” or the like, in the context of methods of synthesis, as used herein refers to reaction conditions, such as time, temperature, solvent, reactant concentrations, and the like, that are within ordinary skill for an experimenter to vary, that provide a useful quantity or yield of a reaction product. It is not necessary that the desired reaction product be the only reaction product or that the starting materials be entirely consumed, provided the desired reaction product can be isolated or otherwise further used.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

An “analog” of a chemical structure, as the term is used herein, refers to a chemical structure that preserves substantial similarity with the parent structure, although it may not be readily derived synthetically from the parent structure. A related chemical structure that is readily derived synthetically from a parent chemical structure is referred to as a “derivative.” In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

At various places in the present specification substituents of compounds of the invention are disclosed in groups or in ranges. It is specifically intended that the invention include each and every individual subcombination of the members of such groups and ranges. For example, the term “C1-C6 alkyl” is specifically intended to individually disclose methyl, ethyl, propyl, isopropyl, n-butyl, sec-butyl, isobutyl, etc. For a number qualified by the term “about”, a variance of 2%, 5%, 10% or even 20% is within the ambit of the qualified number. Standard abbreviations for chemical groups such as are well known in the art are used; e.g., Me=methyl, Et=ethyl, i-Pr=isopropyl, Bu=butyl, t-Bu=tert-butyl, Ph=phenyl, Bn=benzyl, Ac=acetyl, Bz=benzoyl, and the like.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄+ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula (I) compounds, for example in their purification by recrystallization. All of these salts may be prepared by conventional means from the corresponding compound according to Formula (I) by reacting, for example, the appropriate acid or base with the compound according to Formula (I). The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

Each of the terms “halogen,” “halide,” and “halo” refers to —F, —Cl, —Br, or —I.

The term “azide” or “azido” can be used interchangeably and refers to an —N₃ group (—N═N═N) which is bound to a carbon atom and is zwitterionic (carries a + and − charge respectively on the middle nitrogen and the terminal nitrogen). The azide group is a reactant in “click chemistry” which is a copper catalyzed azide-alkyne 1,3 dipolar cycloaddition (Sharpless et al., Angewandte Chemie, 41, 2596 et seq. (2002).

A “hydroxyl” or “hydroxy” refers to an —OH group.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound of the invention can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified, the term “compound” is inclusive in that it encompasses a compound or a pharmaceutically acceptable salt, stereoisomer, and/or tautomer thereof. Thus, for instance, a compound of Formula I includes a pharmaceutically acceptable salt of a tautomer of the compound.

The terms “prevent,” “preventing,” and “prevention” refer to the prevention of the onset, recurrence, or spread of the disease in a patient resulting from the administration of a prophylactic or therapeutic agent.

A “patient” or “subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult.

The term miRNA means a micro RNA sequence that is non-coding for peptides and functions at least for mRNA silencing and post-translational regulation of gene expression. Complementary base pairing of miRNA with messenger RNA molecules manages translation of the mRNA by up and/or down regulation, inhibition, repression and similar translation effects. Typical pre- and pri-miRNA sequences include structured and unstructured motifs. A structured motif is a segment of a pre-miRNA and its embedment within a pri-miRNA having a stable three-dimensional structure that is not wholly dependent upon the particular nucleotide sequence of the structure motif. Hairpin stem, bulge and/or terminal loop regions of pre-miRNA's are typical structured motifs. Groups of miRNAs often cooperate to manage mRNA function. An example is the pri-miRNA-17-92 cluster and the resulting pre-miRNA's and mature miRNA's produced by nuclease action on the cluster and pre-miRNA's respectively.

The terms pri-miRNA and pre-miRNA are the precursor RNA transcripts from which mature miRNA is produced. Transcription of DNA in the cell nucleus produces among other RNA molecules, pri-miRNA, a long RNA sequence which is capped and polyadenylated. Cleavage of the pri-miRNA and RNA chain processing in the nucleus produces the shorter pre-miRNA for export to the cellular cytoplasm. Pre-miRNA is further processed in the cytoplasm by RNAase Dicer to produce double stranded short RNA and one of the two strands becomes mature, single strand miRNA for interaction with messenger RNA.

### DETAILED DESCRIPTION

The lead binding molecular strategy known as Inforna (cited above) was used to design a ligand that targets the primary microRNA-17-92 cluster (pri-miR-17-92), a direct transcriptional target of c-MYC.²¹ This non-coding RNA encodes six different microRNAs (miRNAs): miR-17, -18a, -19a, -19b-1, -20a, and -92a-1. Upregulation of the miR-17-92 cluster has been observed in numerous diseases, from various cancers²²⁻²⁵ to fibrosis.²⁶ Furthermore, the downstream effects are disease-dependent and linked to which members of the cluster are aberrantly expressed.²⁷ Indeed, the miRNAs produced can act individually or synergistically to affect multiple pathways.^{21,28} Thus, this cluster and the pre-miRNAs that comprise it are important targets of chemical probes and lead medicines.

The Inforna sequence-based design approach afforded a single compound (compound 2) that inhibits the biogenesis of three mature miRNAs (miRNA-X's) from the pre-miRNA's embedded in the pri-miRNA-17-92 cluster that share structural similarities, pre-miR-17, pre-miR-18a, and pre-miR-20a. Extension strategies enable a change the compound's mode of action from simple binding to cleavage provided two cleavage strategies: (i) direct, oxidative cleavage and ii) cleavage by recruitment of endogeneous nuclease or a ribonuclease targeting chimera. The first strategy was accomplished by conjugation of bleomycin A5 to the lead compound 2, to produce compound 5. Targeting the pre-miRNA-X's with compound 5 improved potency by ˜10-fold. Compound 5 also degraded the entire pre-miR-17-92 cluster and hence rescued miR-17-92-mediated phenotypes in prostate and triple negative breast cancer (TNBC) cells. The second strategy was accomplished by conjugation of compound 2 with a recruiting moiety for an endogenous nuclease, or a ribonuclease targeting chimera (RIBOTAC). Interestingly, the RIBOTAC inhibited biogenesis of miR-17, -18a, and -20a by binding and cleaving their pre-miRNAs, not the entire cluster, traced to the co-localization of the RIBOTAC, targets, and the endogenous nuclease.

### Design, Optimization, and In Vitro Analysis of Compounds Targeting Pri-miR-17-92

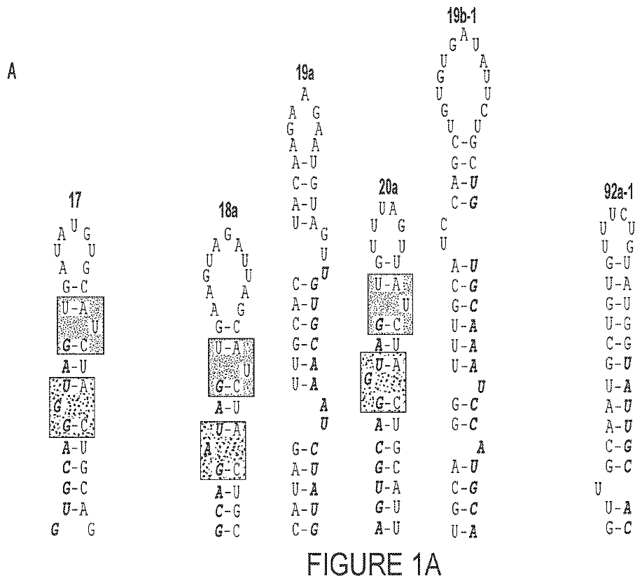

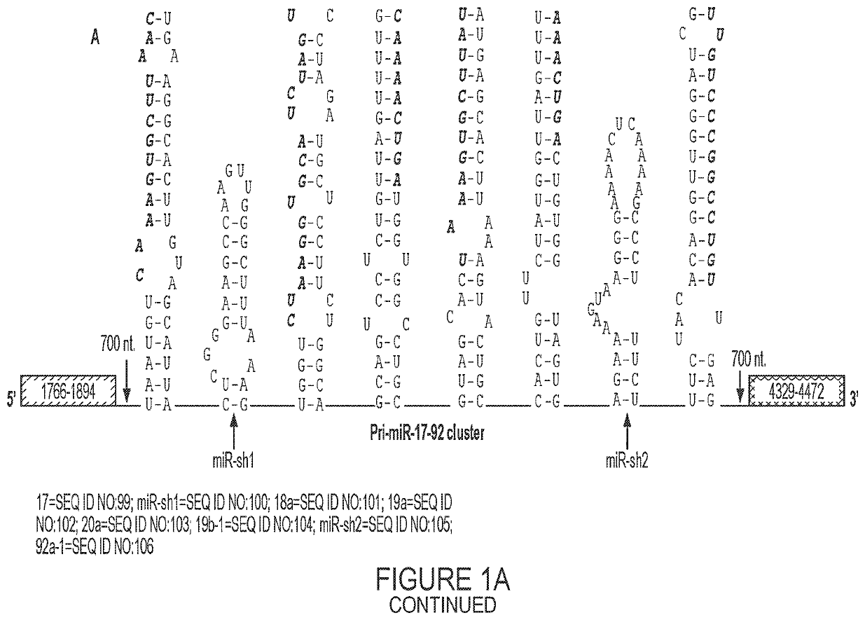

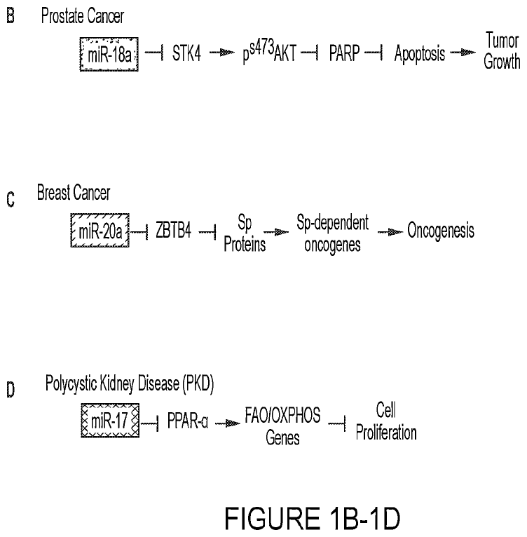

Since the discovery of the miR-17-92 cluster,²⁹ its role in disease has become increasingly apparent and diverse. Upregulation of the miR-17-92 cluster is associated with more than 14 different cancers,²⁷ including osteosarcomas,^{23,30} and retinoblastoma.^{22,31} Elevated levels of one member of the cluster, miR-92a, inhibits angiogenesis in ischemic cardiovascular endothelia,³² while its upregulation in CD4⁺ T cells stimulates an autoimmune response.³³ Consequently, the miR-17-92 cluster is a high priority target for therapeutic intervention. Efforts focused on designing compounds that inhibit the biogenesis of the miR-17-92 cluster by inspecting the structures found at the Drosha and Dicer processing sites of each encoded pre-miRNA. It was previously shown that binding these functional sites inhibits pre-miRNA processing in situ and in vivo.^{3,15,17,18,34,35} Fortuitously, three pre-miRNAs in the pre-miR-17-92 cluster have the same U bulge (5′G_U/3′CUA) in their Dicer sites, pre-miR-17, pre-miR-18a, and pre-miR-20a (FIG. 1). Both pre-miR-17 and pre-miR-20 have an adjacent 1-nucleotide G bulge (5′GGU/3′C_A) while pre-miR-18a has a related purine bulge, 5′GAU/3′C_A (FIG. 1).

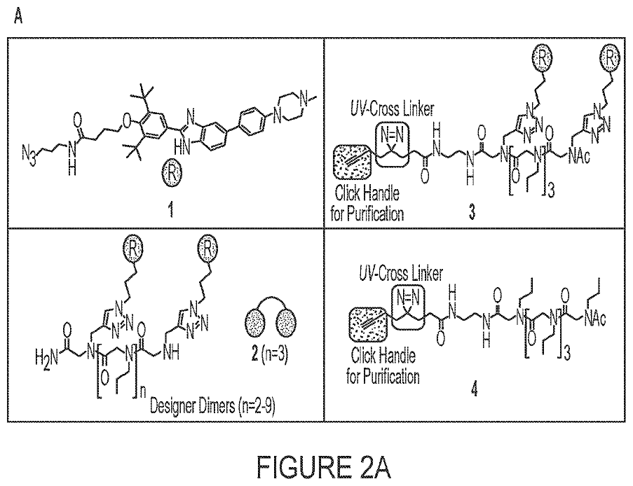

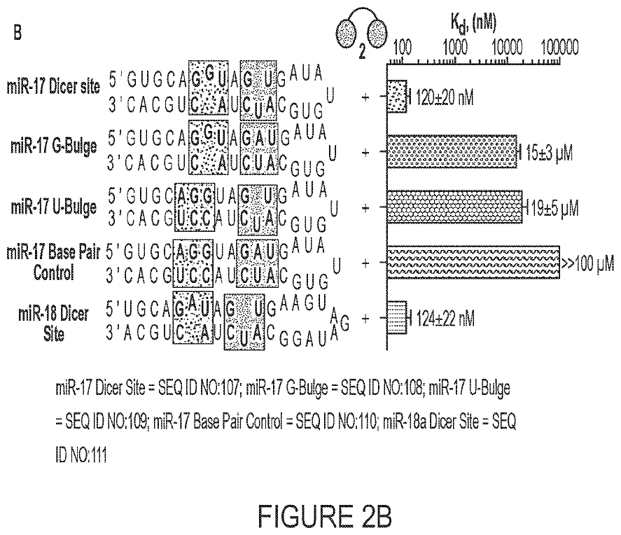

A previous study that explored the RNA-binding capacity of various ligands showed that compound 1² (FIG. 2A) preferred to bind all three of these bulges with K_{d}'s ranging from 30-40 μM.³⁵ No binding was observed between 1 and RNAs in which the bulges were mutated to the corresponding GC or AU base pairs.³⁵ These data suggested a facile route to compound optimization via dimerization of 1 to afford a single molecule that binds both bulges simultaneously.²⁰ Fortuitously, 1 contains a chemical handle for dimerization, an azide moiety that can be conjugated to alkyne-displaying scaffolds via a Cu-catalyzed Huisgen 1,3-dipolar cycloaddition.^{36,37} Compound 1 was conjugated to peptoids displaying two alkynyl groups separated by 2-9 propylamine spacing modules, as informed by previous studies.³⁸ (See the experimental section for synthesis and characterization.) ² In the text of this specification, compounds 1, 2, 4FT, 5, 6, 7 and similarly numbered compounds are frequently designated by the bold number alone without the prefix “compound.” It is understood that a bold number alone and the same number with the prefix “compound” have the same meaning. This meaning is provided by the formulas of these compounds provided in the Summary and in the experimental section of this specification.

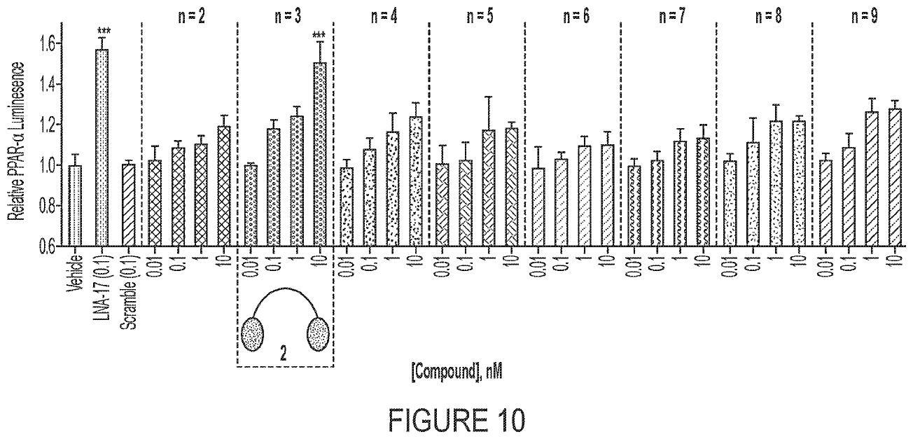

To identify an optimal dimer that displays the RNA-binding modules that most potently targets the pre-miRNAs, the library was screened for activity in a cell-based luciferase reporter assay. It is known that peroxisome proliferator-activated receptor alpha (PPAR-α) mRNA is translationally repressed by miR-17.³⁹ Thus, a construct with luciferase fused to PPAR-α's 3′ untranslated region (UTR)³⁹ was used to assess inhibition of miR-17 biogenesis in HEK293T cells (FIG. 10). A locked nucleic acid (LNA) oligonucleotide targeting miR-17 (LNA-17) was used as a positive control, increasing luciferase activity by 1.6(±0.1)-fold; no effect was observed with a scrambled LNA oligonucleotide (FIG. 10). Of the eight dimers synthesized and tested, the most potent compound contained three propylamine spacers (or compound 2; FIG. 2A), which had an IC₅₀ of ˜10 μM in the luciferase-based assay (FIG. 10). The number of propylamine spacing modules in the optimal compound is in agreement with previous studies for spanning three base pairs between bulges or internal loops.³⁸

### In Vitro Evaluation of 2.

As 2 showed optimal activity amongst the dimers, it was further characterized in vitro and in situ. The affinity of 2 for a model RNA was measured wherein the model contains the two bulges in and adjacent to pre-miR-17's and pre-miR-20a's Dicer site. A 250-fold boost in affinity relative to 1 was observed, affording a K_{d} of 120(±20) nM (FIG. 2B). No measurable binding was detected to RNAs in which the two bulges were mutated to base pairs (FIG. 2B). Single mutations of the bulges, either the U bulge to an AU pair or the G bulge to a GC pair, weakened the avidity of 2 to 15(±3) and 19(±5) M, respectively, ˜2-fold more avid than the binding of 1 which is due to stochastic effects.^{35,40} As expected, based on the similar avidity of 1 for the three bulges in pre-miR-17, -18a, and -20a, 2 exhibited similar affinity for a mimic of pre-miR-18a [124(±22) nM] while no saturable binding to the corresponding fully paired RNA was observed (FIG. 2B).

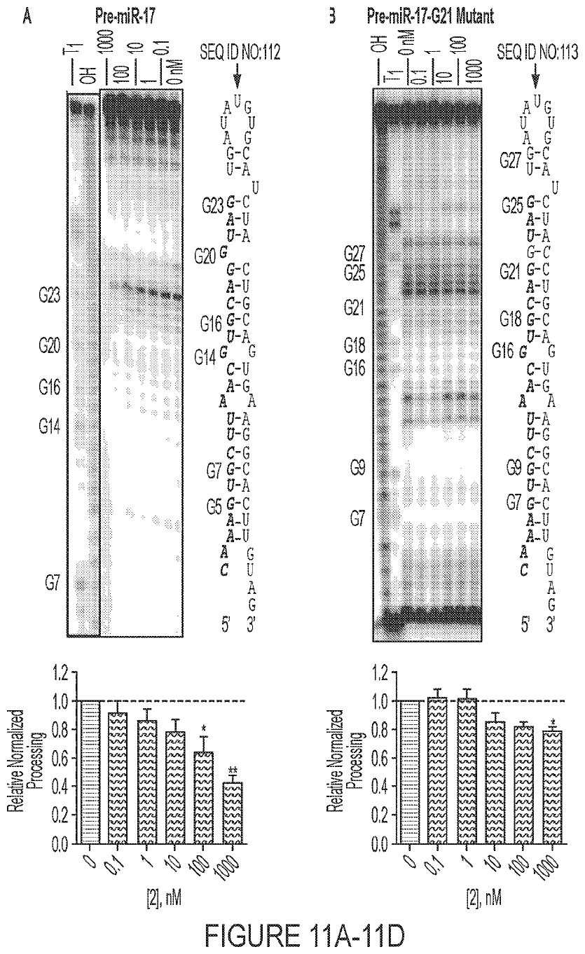

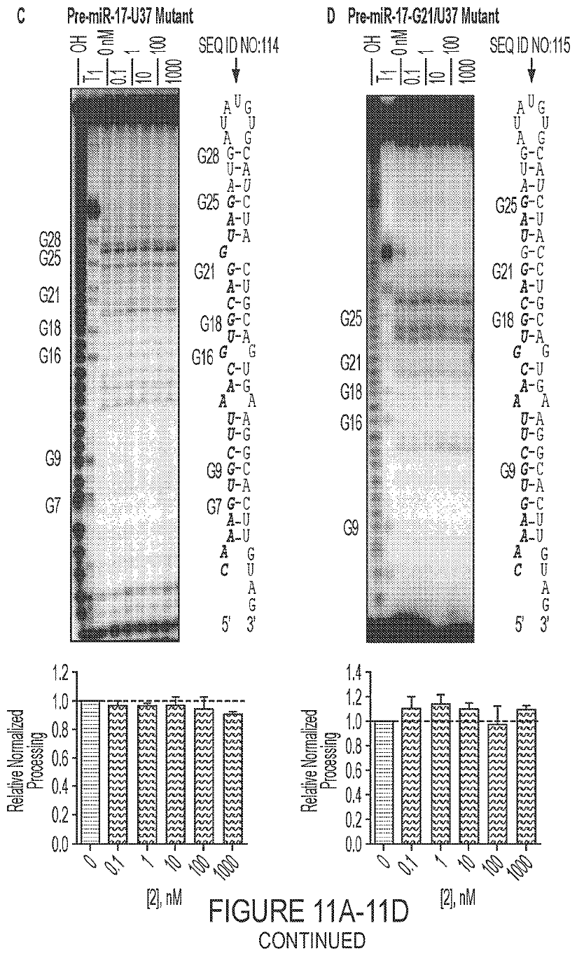

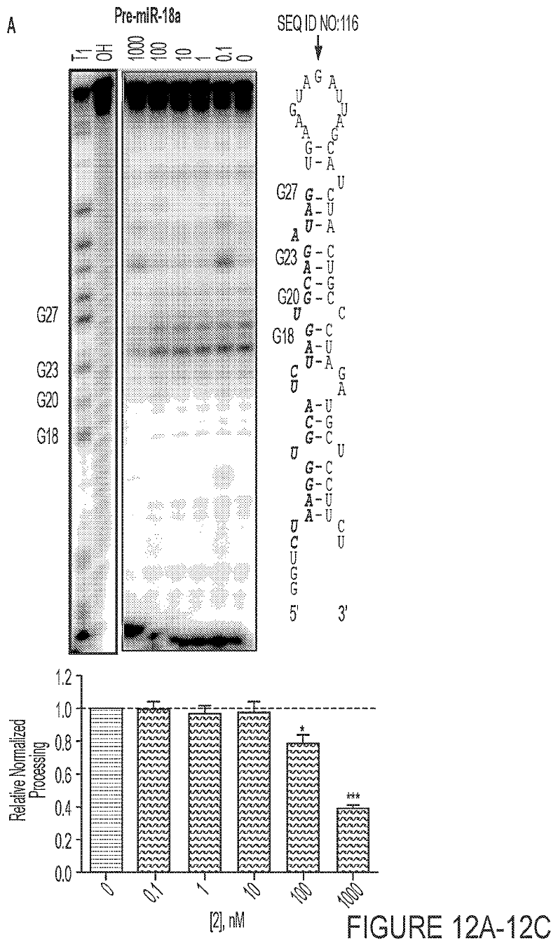

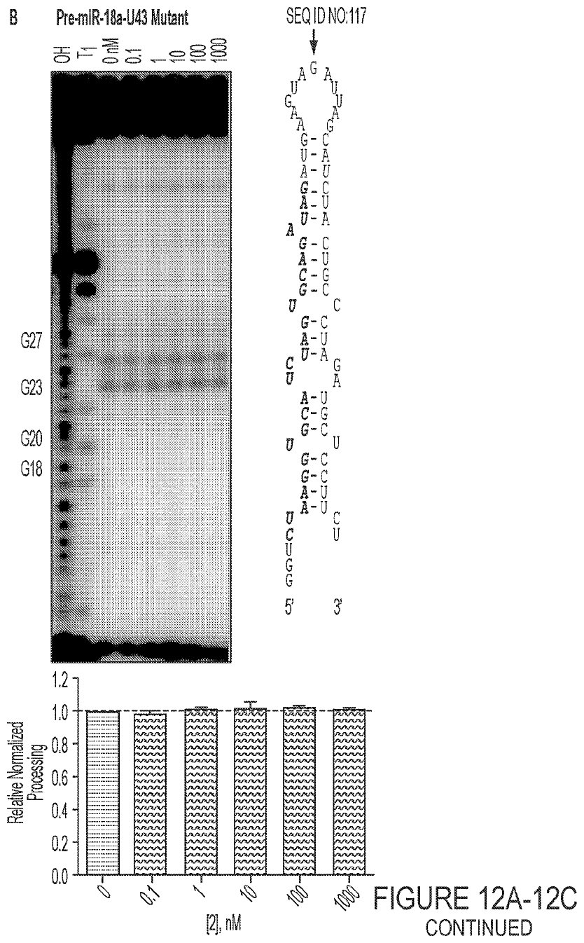

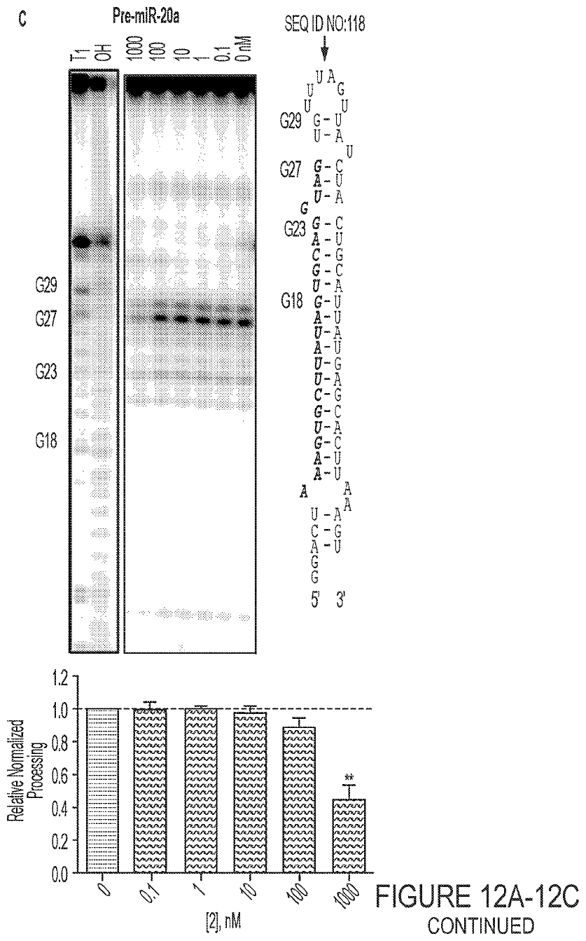

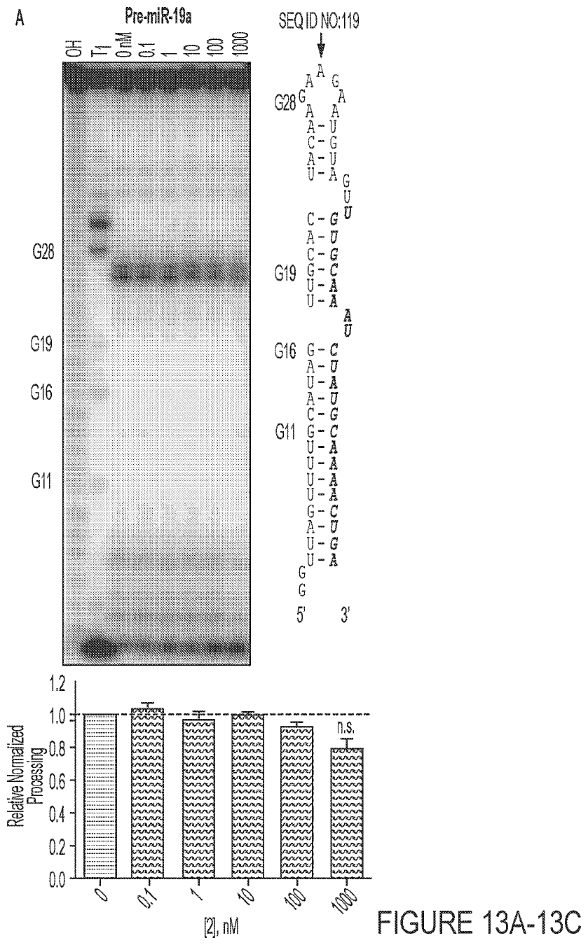

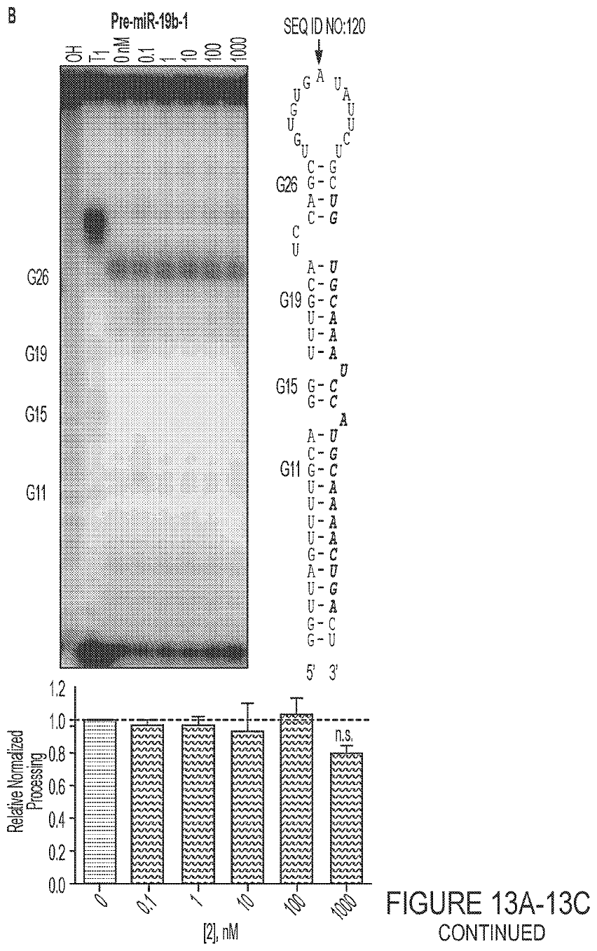

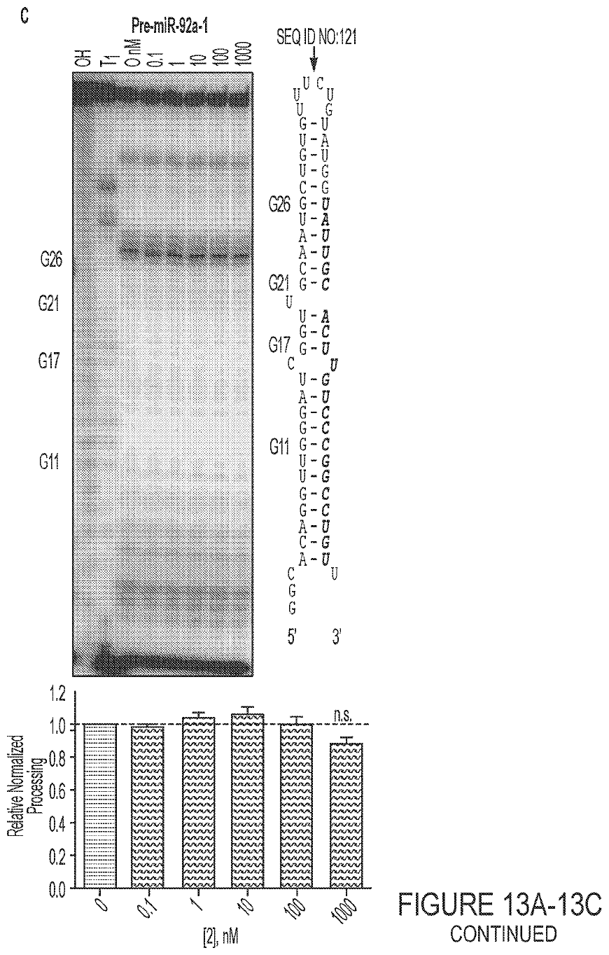

Since 2 bound model constructs of pre-miR-17, pre-miR-18a, and pre-miR-20a with similar affinity, its ability to inhibit Dicer processing of all three pre-miRNAs in vitro was studied. As expected, 2 inhibited Dicer processing of each to a similar extent, with an IC₅₀ of ˜1 M (FIGS. 11A-11D, & 12A-12C). To study the effect on 2's binding site within pre-miR-17 and as a secondary assessment of selectivity (binding studies being the first), a mutational analysis was completed in which each bulge in and nearby the Dicer sites was individually and then collectively replaced with the corresponding base pair. These changes were tolerated by Dicer (FIGS. 11A-11D). The mutations, however, significantly reduced (in the case of the G to GC pair mutation) or ablated (in the cases of the U to AU pair mutation and the double mutant) 2's inhibitory activity (FIGS. 11A-11D). Likewise, when the U bulge in pre-miR-18a was converted to an AU pair, 2 was unable to inhibit Dicer processing at the concentrations tested, up to 1 μM (FIG. 12B). Further, 2 had no effect on the Dicer processing of the other three miRNAs in the cluster, pre-miR-19a, pre-miR-19b-1, or pre-miR-92a-1 (FIGS. 13A-13C). Altogether, these studies support that 2 is a specific RNA-binding compound, targeting the Dicer site and an adjacent bulge in pre-miR-17, pre-miR-18a, and pre-miR-20a and that 2 does not inhibit Dicer itself.

### Effect of 2 on the Pri-miR-17-92 Cluster in TNBC Breast Cancer Cells.

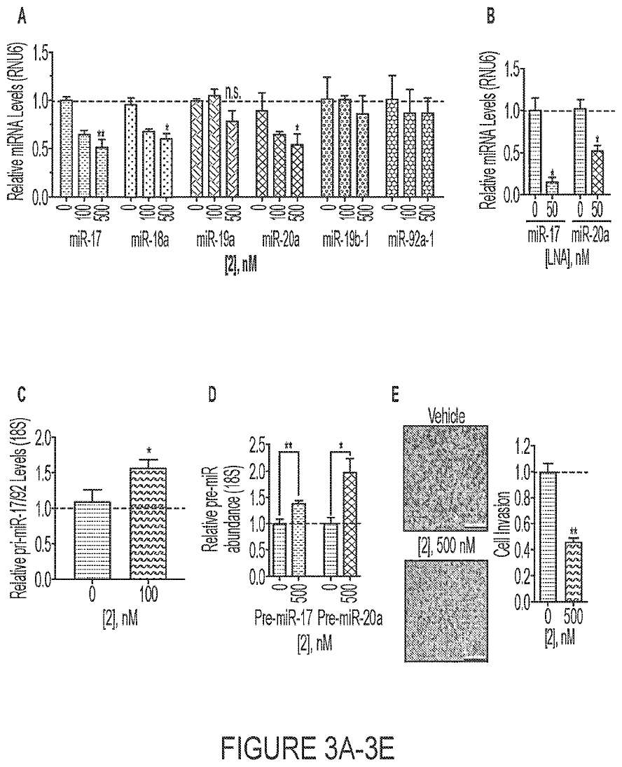

In the TNBC cell line MDA-MB-231, miR-17 and miR-20a are highly expressed and together silence zinc finger and BTB domain containing 4 (ZBTB4) mRNA, thereby triggering an invasive phenotype (FIG. 1C).²⁴ Notably, miR-17 and miR-20a have identical seed sequences, or the sequences that form complexes with targeted mRNAs, to repress their translation (miR-17: 5′-AAAGUGC; miR-20a: 5′-AAAGUGC). Thus, both miR-17 and miR-20a inhibit ZBTB4 in TNBC cells and knockdown of both is necessary to allow robust activation of ZBTB4's tumor suppressor functions. Loss of ZBTB4 has been correlated with shorter relapse-free survival of breast cancer patients making ZBTB4 a therapeutically important target.²⁴ Thus, 2 was studied to determine whether it could inhibit biogenesis of the targeted miRNAs and an invasive phenotype in MDA-MB-231 cells. Compound 2 selectively decreased the levels of mature miR-17, -18a, and -20a dose dependently, while not affecting the levels of the other miRNAs in the cluster (FIG. 3A). The reduction in mature miRNA levels by 2 at 500 nM (˜40-50%) was similar to the reduction observed for an LNA (50 nM) targeting the mature sequence of miR-20a (48(±6)%) but less than that observed for miR-17 (85(±5)%) (FIG. 3B).

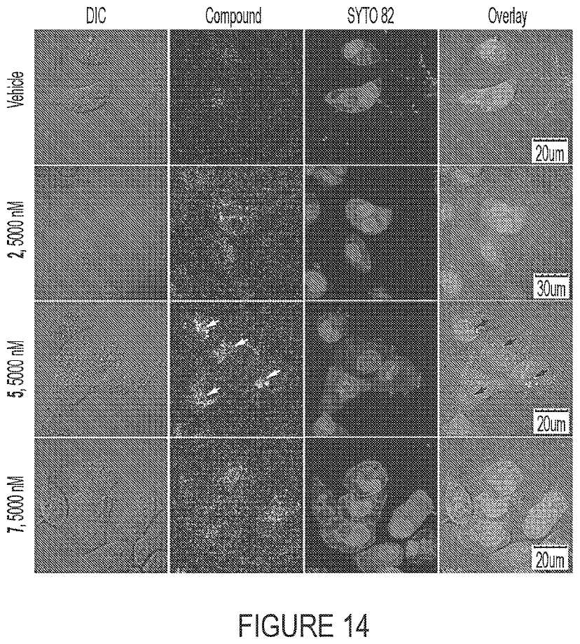

Compound 2 has a significant effect on pri-miR-17-92, pre-miR-17, and pre-miR-20a levels. Depending on its cellular localization, 2 could engage the pri-miRNA or the pre-miRNAs to reduce mature miRNA levels. It is known that the 17-92 cluster folds into a compact tertiary structure, and that alterations in this structure affects the processing of the pri-miR-17-92 transcript.^{41,42} Although 2 localized mainly to the cytoplasm, fluorescence was also detected in the nucleus, suggesting that it could inhibit processing of the pri-miRNA as well as pre-miR-17, -18a, and 20a (FIG. 14). Indeed, 2 increased pri-miR-17-92 levels by 50(±6)%, as determined by RT-qPCR using primers upstream of all six miRNAs (p<0.05; FIG. 3C). [Notably, direct engagement of pri-miR-17-92 by 2 was confirmed via a competition experiment between 2 and a 2-bleomycin conjugate in which 2 restored pri-miR-17-92 levels (vide infra).] Thus, the increase in pri-miR-17-92 levels observed is due to engagement of the primary transcript in the nucleus, likely affecting its overall structure and its global processing.

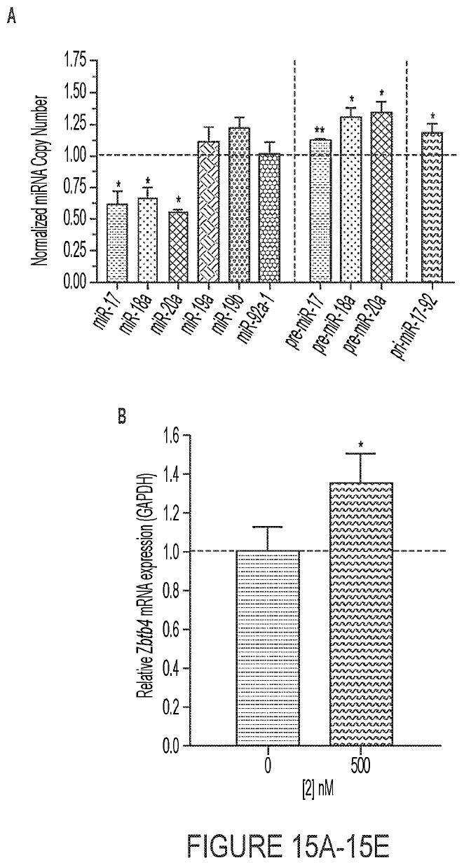

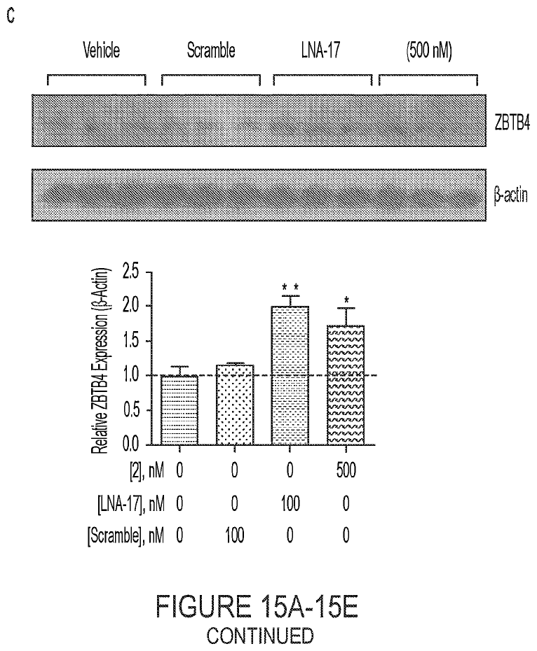

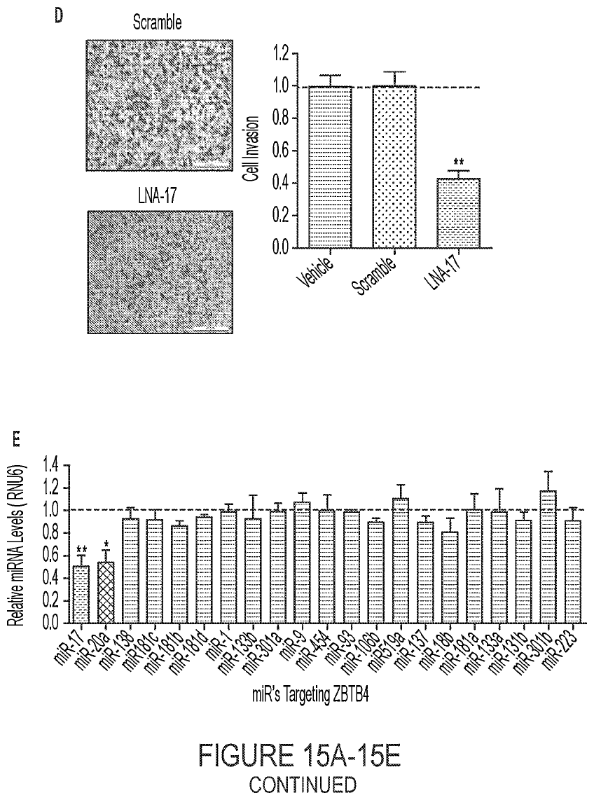

If 2 only engaged pri-miR-17-92 and inhibited its biogenesis, then a decrease in pre-miRNA levels is expected. If, however, 2 directly binds and inhibits processing of both the pri- and pre-transcripts, two outcomes are possible: (i) no change in pre-miRNA levels are observed. That is, the reduction in pre-miRNA levels due to inhibition of pri-miRNA processing and the boost in pre-miRNA levels due to inhibition of Dicer processing are similar and thus cancel each other out; or (ii) an increase in pre-miRNA levels is observed because Dicer processing is inhibited to a greater extent than Drosha processing. In accordance with the last possibility and its presence in the cytoplasm (FIG. 14), 2 increased levels of pre-miR-17 by 1.4(±0.04)-fold (p<0.01) and pre-miR-20a by 2.0(±0.2)-fold (p<0.05) (as determined by RT-qPCR using primers specific for each pre-miRNA; FIG. 3D). [Note: RT-qPCR primers that amplify pre-miRNAs cannot distinguish between the processed, cytoplasmic pre-miRNA and the pre-miRNA in the context of the pri-miRNA; thus, the increase observed in a combined effect of changes in the levels of the cluster and the pre-miRNA of interest.] These results were confirmed by absolute quantification of mature, pre- and pri-miR-17-92 levels (FIG. 15A).

Compound 2 also has an effect on miR-17 and -20a's downstream target ZBTB4.²⁴ Indeed, 2 increased Zbtb4 mRNA levels by 1.4(±0.2)-fold at 500 nM (p<0.05) (FIG. 15B) and ZBTB4 protein levels by 1.7(±0.2)-fold (p<0.05) at 500 nM, similar to the effect observed for 100 nM LNA-17 (FIG. 15C). Previous studies have shown that de-repression of ZBTB4 via inhibition of miR-17 and miR-20a decreases the invasive properties of breast cancer cells.²⁴ We therefore studied if 2 could rescue this phenotype in TNBC cells. Similar to LNA-17 treatment (FIG. 15D), 2 inhibited the invasive characteristics of MDA-MB-231 cells (FIG. 3E). Interestingly, this effect can be traced to inhibition of miR-17 and miR-20a biogenesis by 2 as levels of other miRNAs predicted to regulate expression ZBTB4 via TargetScan were unaffected (FIG. 15E).⁴³ These data support that the observed rescue of phenotype is via 2's inhibition of the miR-17/20a-ZBTB4 circuit.

### Effect of 2 on the Pri-miR-17-92 Cluster in Autosomal Dominant Polycystic Kidney Disease (ADPKD).

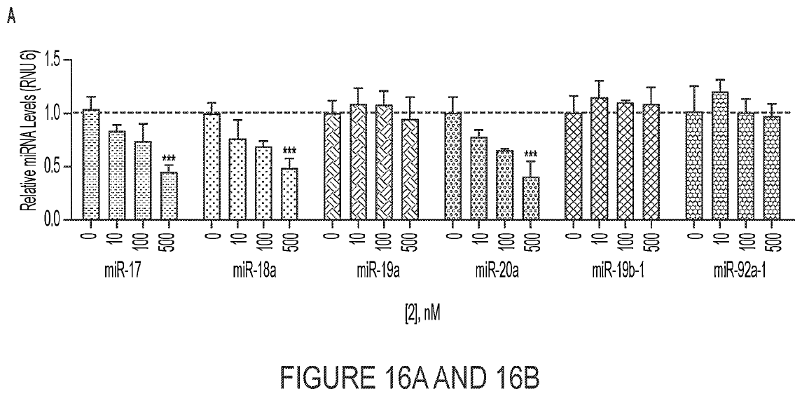

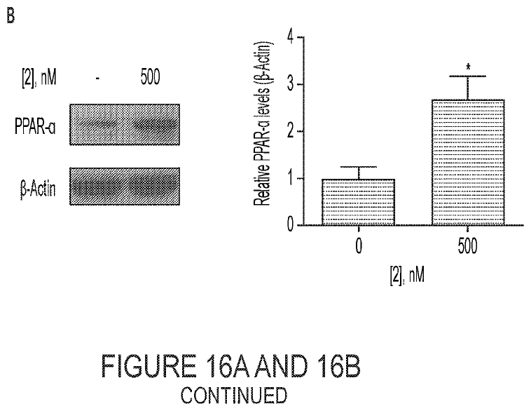

Autosomal dominant polycystic kidney disease is the most common genetically-defined kidney disease, ultimately leading to renal failure.³⁹ Recent studies have shown that miR-17, -19a, -19b-1, and -20a from the miR-17-92 cluster are upregulated in cystic kidneys, which in turn repress PPAR-α and aggravate cyst growth (FIG. 1D).³⁹ Thus, the effect of 2 was evaluated in WT 9-12 cells, an immortalized cell line generated from renal epithelial cell from proximal and distal tubules of ADPKD patients.⁴⁴ Similar to our studies in DU-145 (vide infra) and MDA-MB-231 TNBC cells, 2 decreased expression of its cognate targets, miR-17, miR-18a, and miR-20a, dose dependently (by 55(±6)%, 52(±9)%, and 59(±13)%, respectively, at 500 nM), with no effect on miR-19a, miR-19b-1, and miR-92a-1 (FIG. 16A). As expected, reduction of mature miR-17 levels by 2 de-repressed PPAR-α expression, increasing protein levels by 2.7-fold at 500 nM (FIG. 16B). Importantly, these studies show that reduction of miR-17 and miR-20a levels alone are sufficient to derepress PPAR-α, as levels of miR-19a and -19b-1, which also regulate PPAR-α,³⁹ were unaffected by 2 treatment (FIG. 16A).

### Effect of 2 on the Pri-miR-17-92 Cluster in the Prostate Cancer Cell Line DU-145.

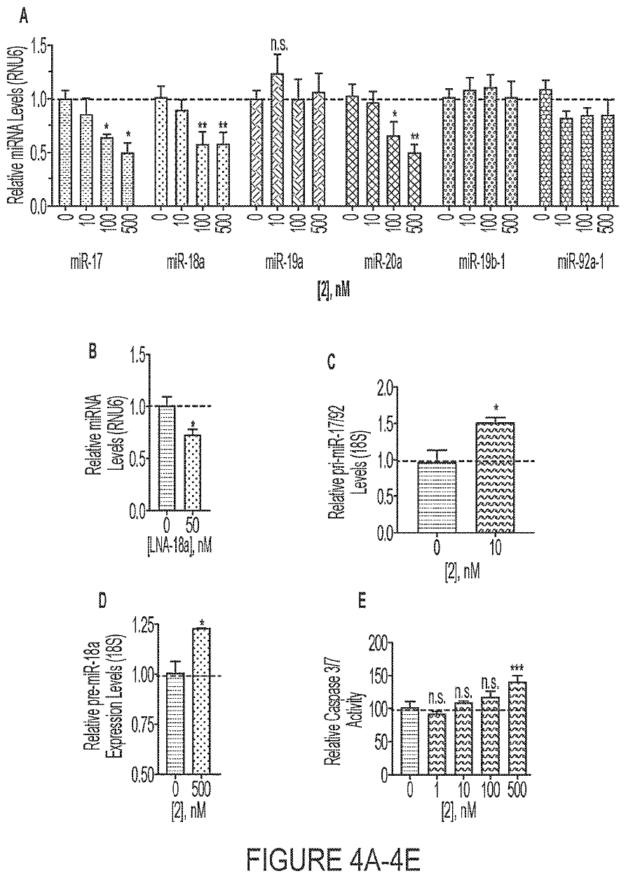

The pri-miR-17-92 cluster, particularly by the overexpression of miR-18a, promotes prostate cancer.⁴⁵ Consequently it is expected that compound 2 will exhibit an inhibitory effect on the biogenesis of the miR-17-92 cluster in the prostate cancer cell line DU-145. As expected, based on its in vitro binding affinity and activity, application of 2 inhibited the biogenesis of miR-17, -18a, and -20a biogenesis, decreasing mature miRNA levels of each (FIG. 4A). As little as 100 nM of 2 was sufficient to significantly inhibit production of the mature miRNAs: by 49(±9)% for miR-17 (p<0.05); by 49(±8) for miR-20a (p<0.05); and by 58(±11)% for miR-18a (p<0.01) (FIG. 4A). Importantly, 2 did not affect levels of miR-19a, -19b-1, and -92a-1, which it does not bind. LNA-18a reduced miR-18a levels by 27(±5)% (FIG. 4B). Compound 1, the monomer from which 2 is derived, was previously studied for reducing miR-18a levels in DU-145 cells.³⁵ The reduction in mature miR-18a levels observed upon treatment with 100 nM of 2 is similar to that upon treatment with 10 □M of 1,³⁵ or a 100-fold increase in potency.

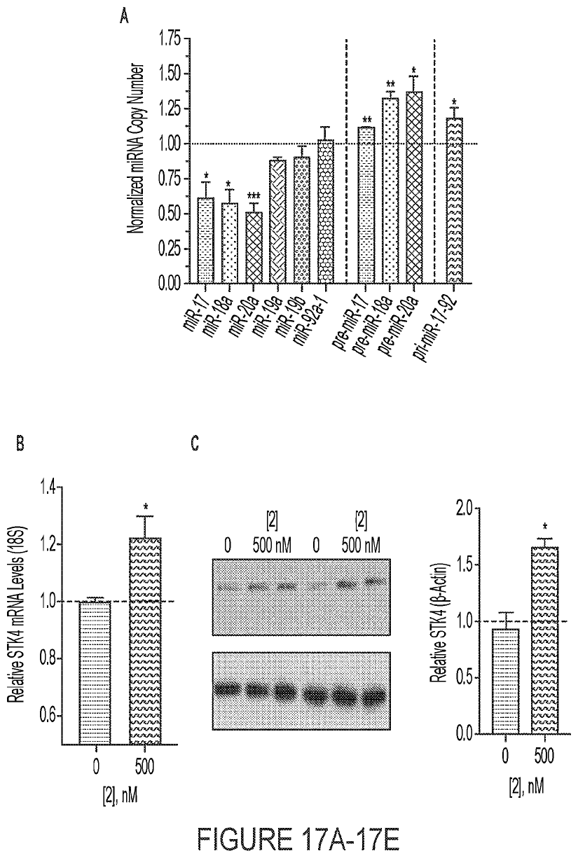

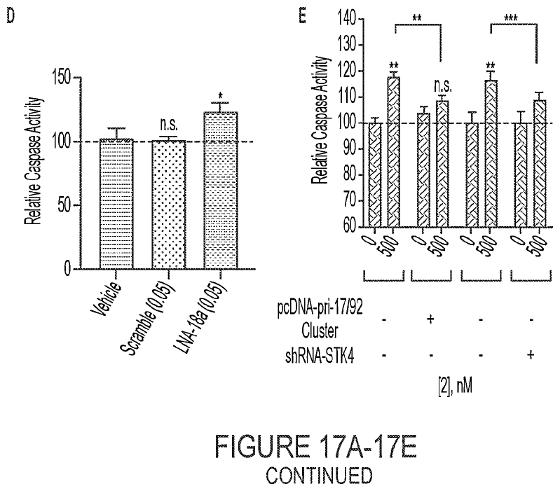

Consistent with studies in MDA-MB-231 TNBC cells, 2 increased levels of pri-miR-17-92 by 51(±6)% and pre-miR-18a by 23(±0.05)% (FIGS. 4C and D). These results, in conjunction with the decreased levels of mature miR-18a, support the hypothesis that 2 inhibits processing of the pri- and pre-miRNA. To validate these observations, the effects of compound 2's treatment on mature and pre-miR-17, -18a, and -20a was well as pri-miR-17/92 levels by absolute quantification, which is in agreement with relative quantification data (FIG. 17A).

### Downstream Effects of 2 in DU-145 Prostate Cancer Cells.

In DU-145 cells, miR-18a translationally represses serine/threonine-protein kinase 4 (STK4).⁴⁵ A previous study showed that an antagomir directed against miR-18a increased STK4 protein levels and triggered apoptosis via STK4-mediated dephosphorylation of protein kinase B [also known as AKT serine/threonine kinase (AKT)]. Treatment of DU-145 cells with 2 increased levels of Stk4 mRNA by 22(±7)% (FIG. 17B) and STK4 protein by 65(±7)% (FIG. 17C). To determine whether 2 can trigger apoptosis, Caspase 3/7 levels were measured upon 2 treatment. Indeed, compound 2 triggered apoptosis, increasing Caspase 3/7 activity by 41(±8)% at 500 nM (FIG. 4E). An LNA targeting miR-18a increased Caspase 3/7 activity by 24(±8)% while no effect was observed for a scrambled control (FIG. 17D).

To confirm that 2 triggered apoptosis by the miR-18a-STK4 circuit, we (i) overexpressed pri-miR-17-92 via a plasmid and (ii) knocked down STK4 via an shRNA. As expected, both overexpression of the cluster and knock down of its downstream target (STK4) reduced 2's ability to trigger apoptosis (FIG. 17E). The activity of 2 was unaffected upon transfection of a control plasmid or scrambled shRNA (FIG. 17E). These observations support the hypothesis that 2 induced apoptosis in a miR-17-92 cluster- and STK4-dependent manner.

Collectively, these studies show that 2's inhibition of the processing of three pre-miRNAs derived from the pri-miR-17-92 de-represses their downstream targets to alleviate oncogenic phenotypes in two different cellular models of disease, prostate cancer and breast cancer. Compound 2 also shows promising activity in an ADPKD model and will be the subject of further investigation.

### Compound 2 Directly Engages the Pri-miR-17-92 Cluster In Situ, as Determined by Chem-CLIP.

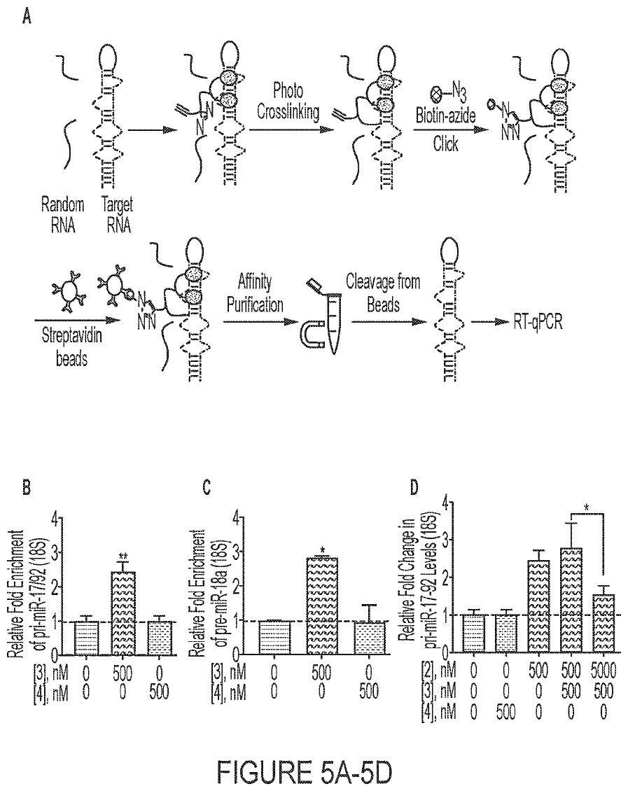

A previously developed method named Chemical Cross-Linking and Isolation by Pull-down (Chem-CLIP) was used to assess the direct target engagement of 2 (FIG. 5A).^{35,46,47} To enable these studies, 2 was equipped with a diazirine module, a photo cross-linking group that reacts with nucleic acids upon irradiation, and a terminal alkyne that can react with biotin-azide, used for isolation and purification (compound 3, FIG. 2A). A control compound 4, which lacks the RNA-binding modules but retains the reactive diazirine and alkyne on the peptoid backbone, was also synthesized (FIG. 2A).

DU-145 cells were treated with 500 nM of 3, followed by isolation of 3-RNA adducts. A 2.4-fold enrichment of pri-miR-17-92 was observed, as compared to its levels in the lysate prior to pull-down, while no significant enrichment was observed for cells treated with 4 (FIG. 5B). Interestingly, these results suggest that 3 directly engaged the primary transcript in the nucleus. Enrichment was also measured using RT-qPCR primers complementary to pre-miR-18a, as overexpression of miR-18a represses apoptosis in prostate cancer cells via STK4. Indeed, 3 enriched pre-miR-18a by 2.8-fold enrichment in Chem-CLIP studies; no significant enrichment of pre-miR-18a was observed with 4 (FIG. 5C). Finally, to further confirm that the parent compound 2 engages the same sites as the Chem-CLIP probe 3, a competition experiment (C-Chem-CLIP) was completed by pre-treating DU-145 cells with varying concentrations of 2, followed by dosing with 3. Indeed, these studies showed that 2 reduced the amount of pri-miR-17-92 pulled down by 3 in a dose-dependent manner, demonstrating that the two compounds bind the same sites (FIG. 5D).

### Targeted Cleavage of the Pri-miR-17-92 Cluster.

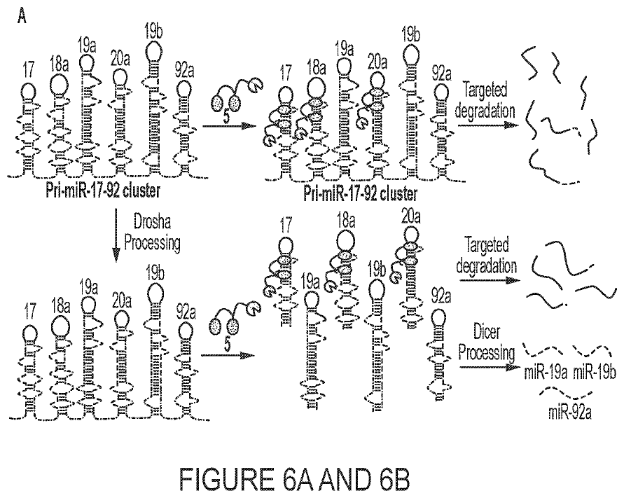

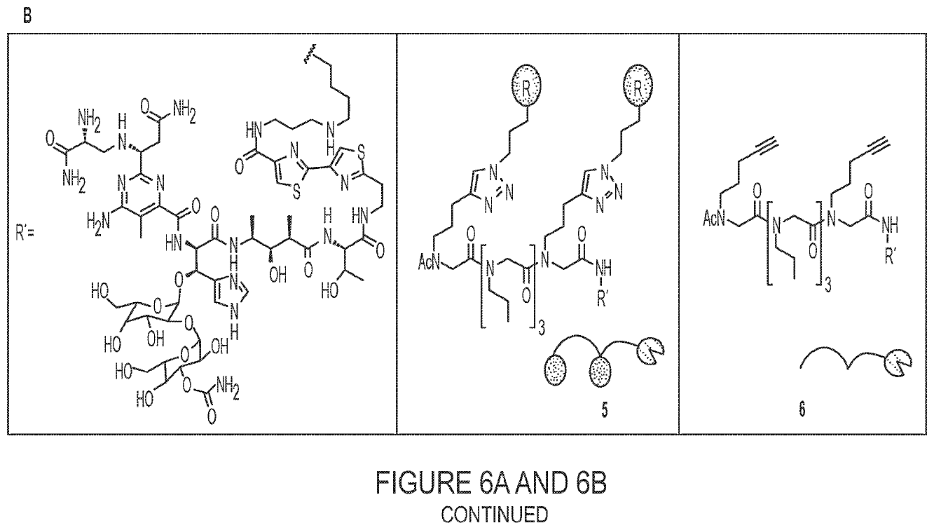

Based on cellular localization, RT-qPCR, and Chem-CLIP analyses, 2 inhibited the biogenesis of only those miRNAs that it bound, namely miR-17, miR-18a, and miR-20a. Since all members of the miR-17-92 cluster are implicated in disease, it would be desirable to have a compound that inhibits generation of all mature miRNAs in the cluster (FIG. 6A).²⁷ The destruction of precursor and primary transcripts was studied by equipping 2 with a bleomycin A5 cleaving module. Conjugation of bleomycin A5 to an RNA-binding compound has been shown to allow for the programmable cleavage of the desired RNA target in cells and in an animal model.^{19,48,49} That is, this approach can direct cleavage selectively to RNA targets with which the RNA-binding compounds interact while directing cleavage away from DNA, bleomycin's canonical target. RNA-selective cleavage is afforded by conjugation of bleomycin A5's free amine, which contributes to the recognition of DNA.^{19,49,50} Thus, a 2-bleomycin A5 conjugate, 5, and its control compound 6, which lacks RNA-binding modules, were synthesized (FIG. 6B).

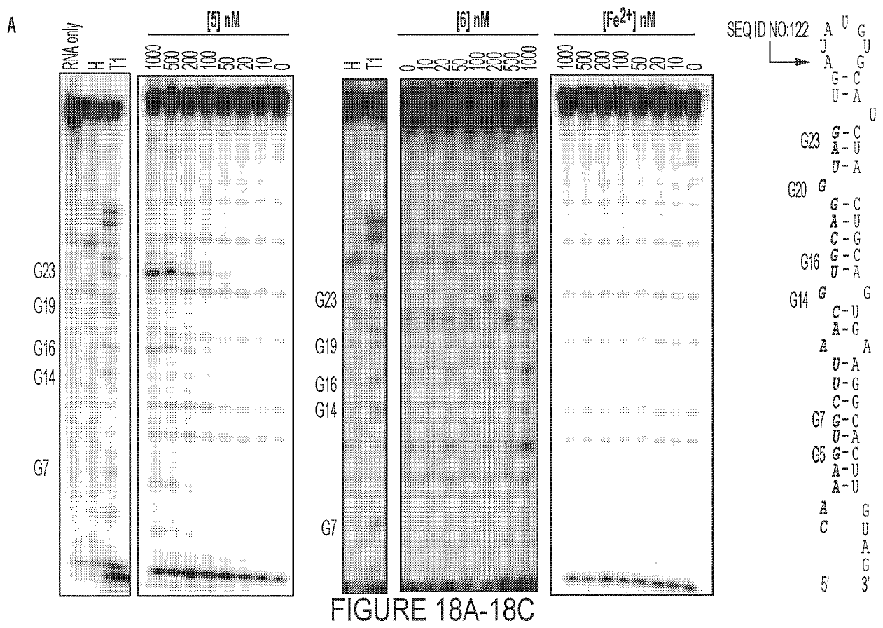

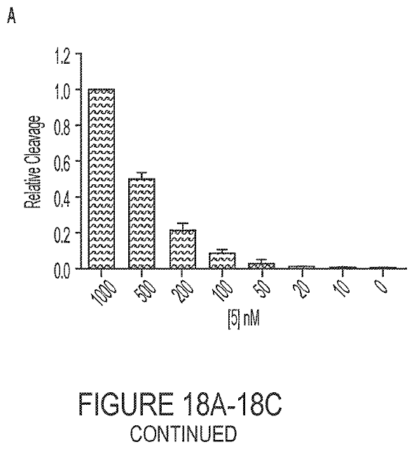

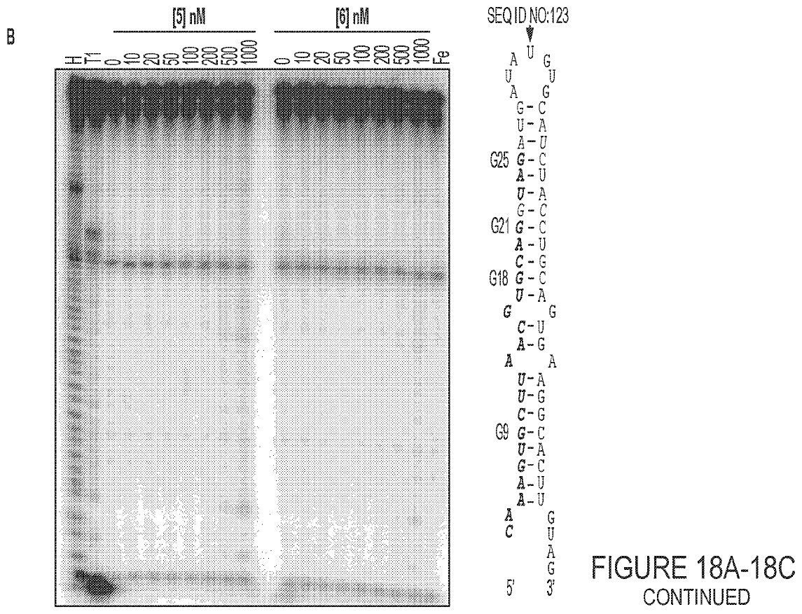

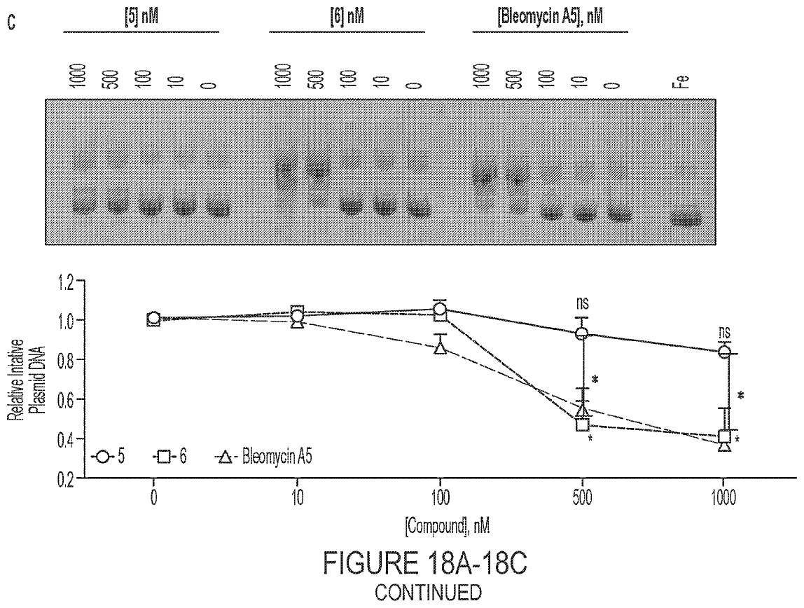

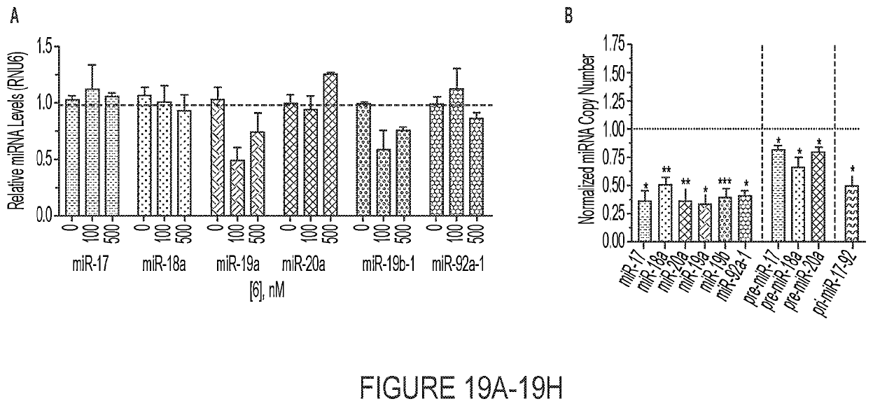

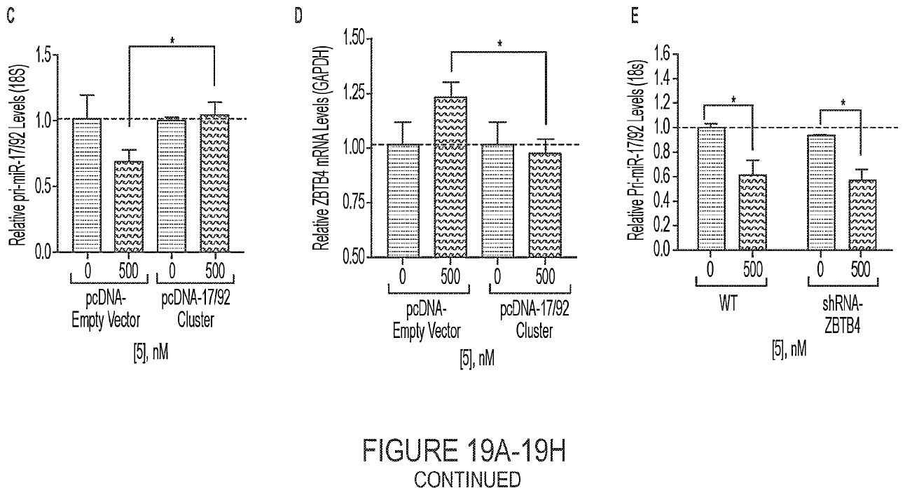

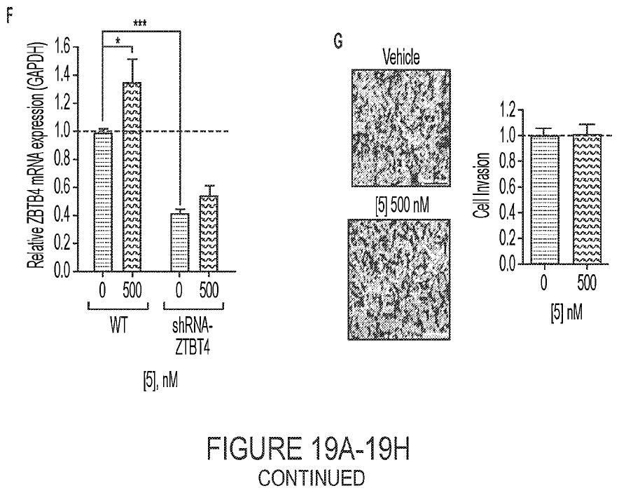

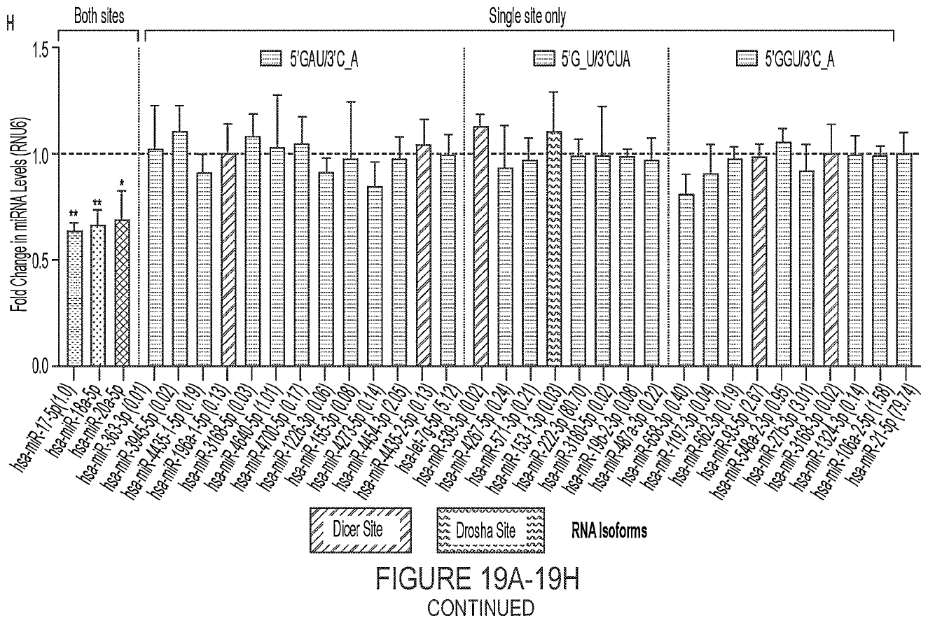

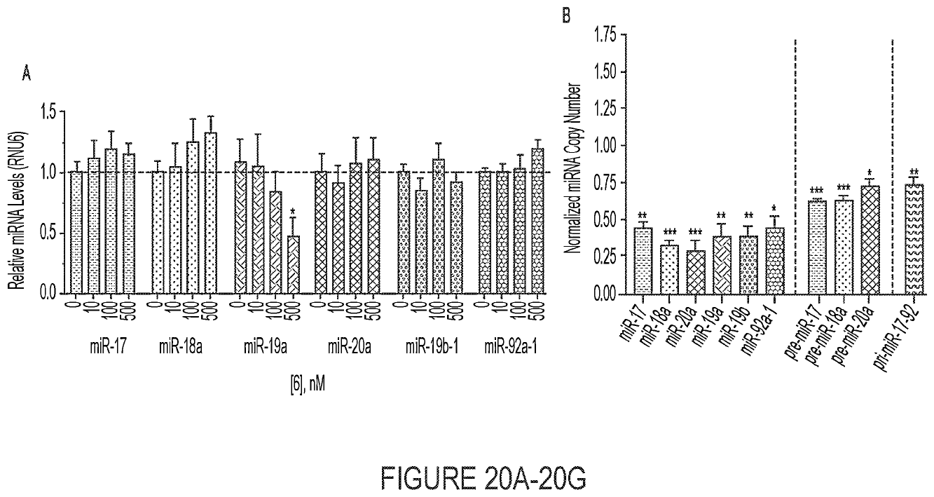

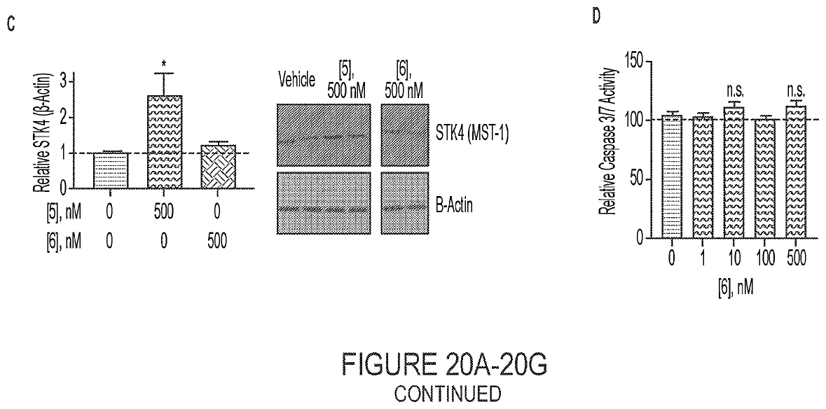

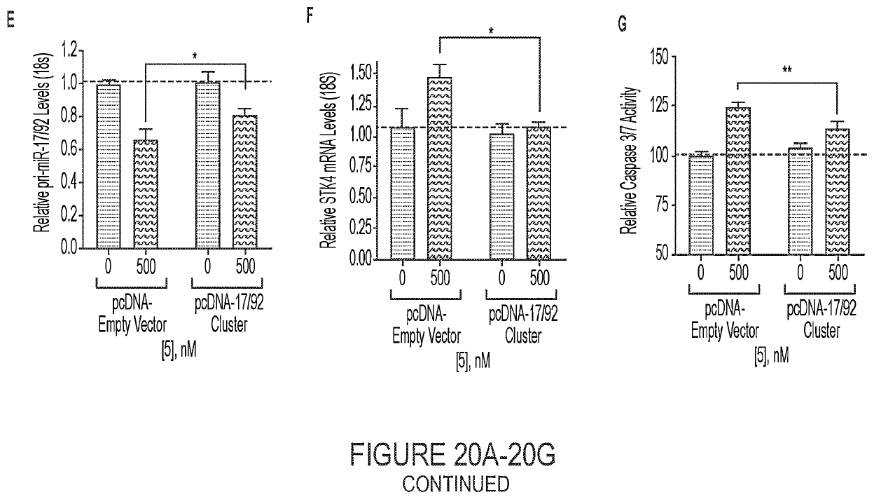

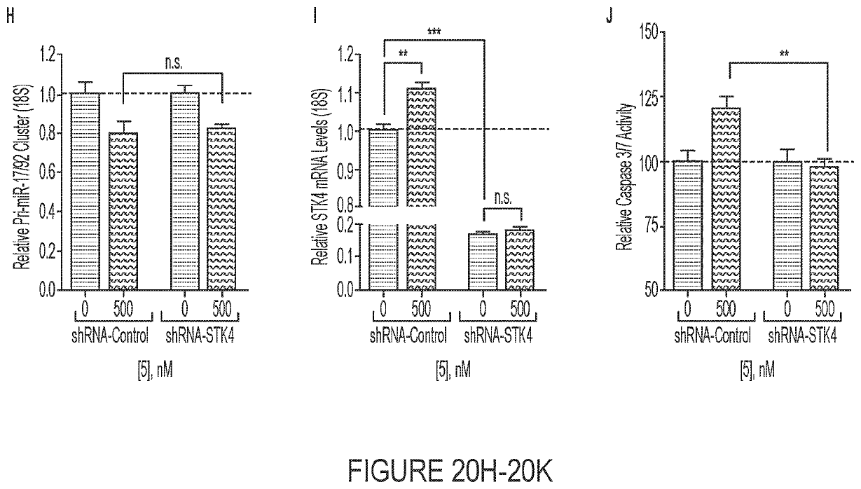

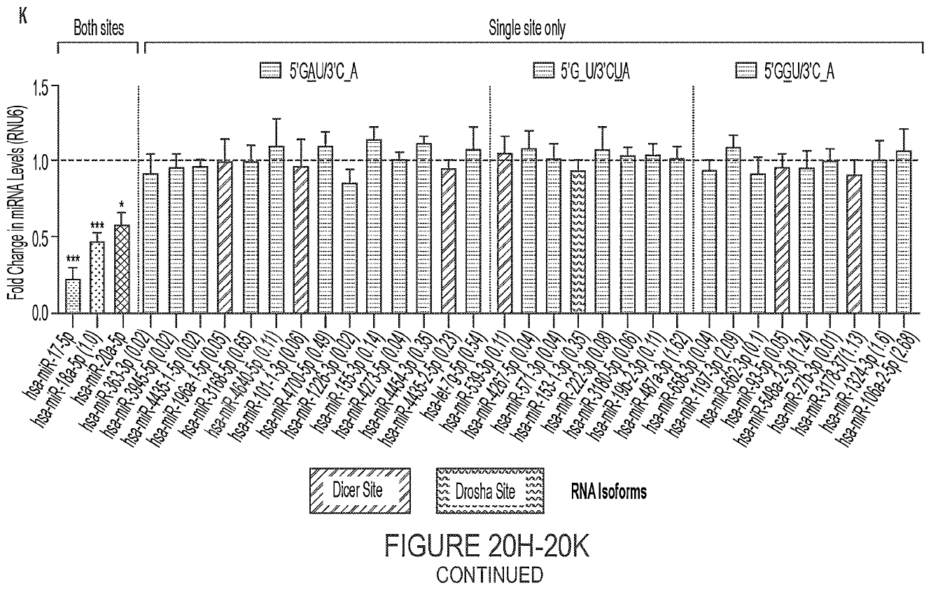

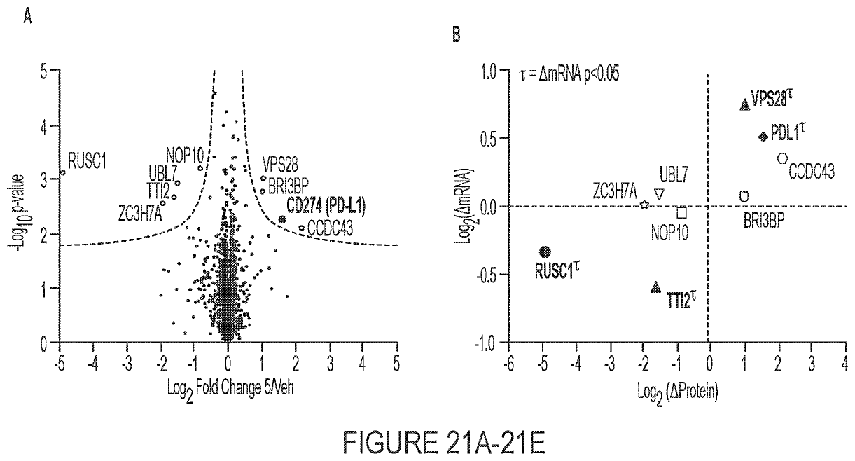

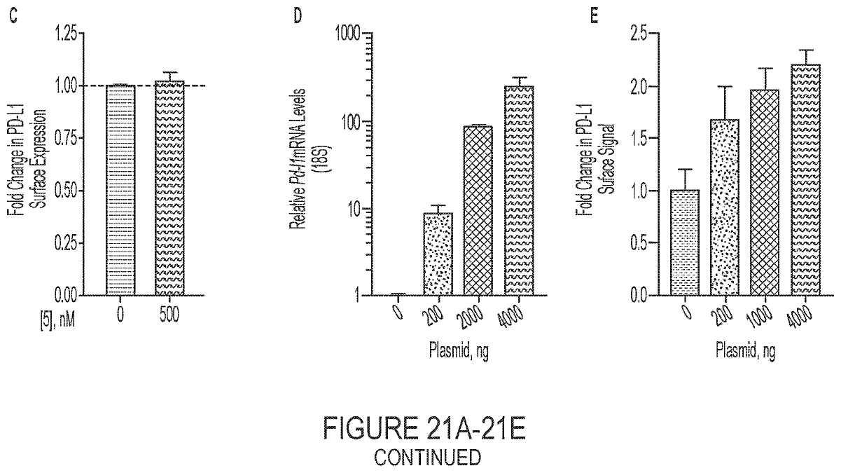

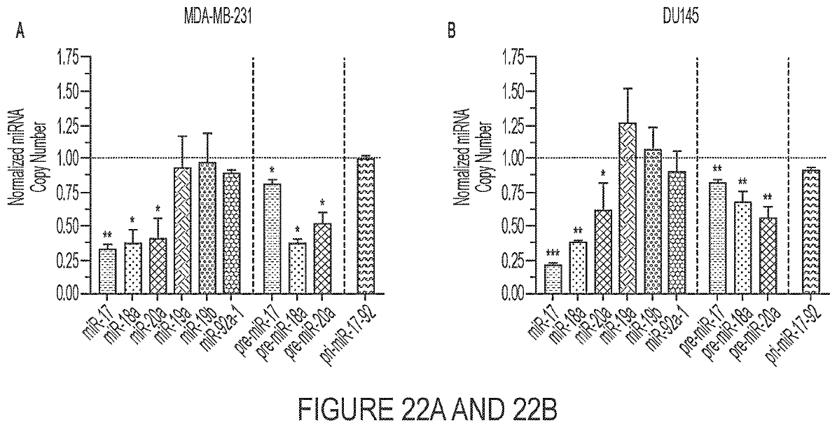

Compound 5 was assessed for its ability to cleave pre-miR-17 in vitro. As expected, based on our in vitro binding and Dicer processing studies (FIGS. 2B and 11A-11D), 5 specifically cleaved pre-miR-17 nearby its Dicer processing site in a dose dependent manner with an IC₅₀ of ˜500 nM. (FIG. 18A). In contrast, no dose-dependent cleavage was observed with control compound 6, which lacks RNA binding modules (FIG. 18A). When the U bulge at the Dicer site and the adjacent G bulge were mutated to AU and GC base pairs, respectively, no cleavage due to the compound was detected (FIG. 18B). These results collectively demonstrate specific binding of 5 at the designed site, the Dicer processing site and adjacent bulge. Although conjugation of bleomycin A5's amine to RNA-binding molecule has been previously shown to reduce DNA cleavage overall and ablate cleavage at active concentrations that cleave the desired RNA target,^{19,49,50} 5 or 6 were assessed to determine whether either cleaved plasmid DNA to a greater extent than bleomycin A5 itself. Bleomycin A5 significantly cleaved DNA with as little as 100 nM of compound (FIG. 18C), while at the same concentration 5 did not significantly cleave the DNA (FIG. 18C). Compound 6 had attenuated DNA cleavage capacity relative to parent bleomycin A5 at 100 nM concentration (FIG. 18C). Thus, the compound 5 is an RNA-specific cleaver in vitro.

### Cleavage of the Pri-miR-17-92 Cluster by 5 in MDA-MB-231 TNBC Cells.

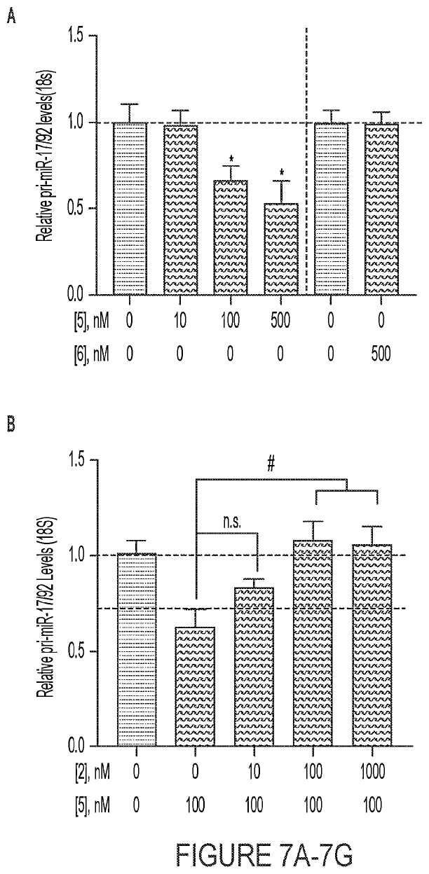

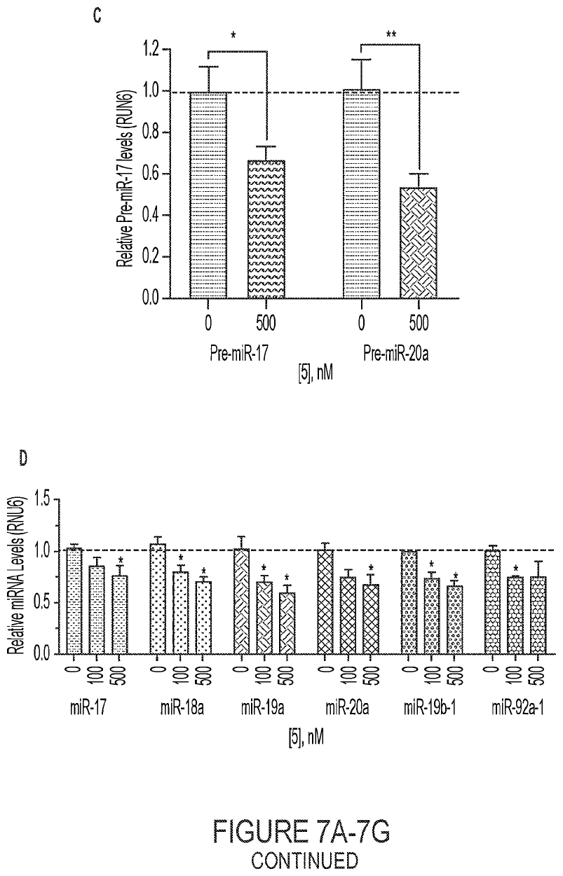

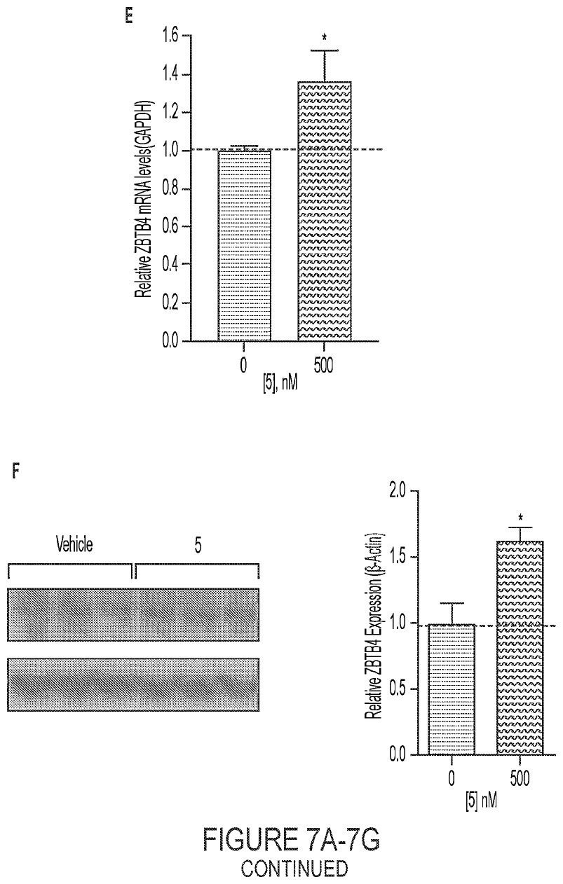

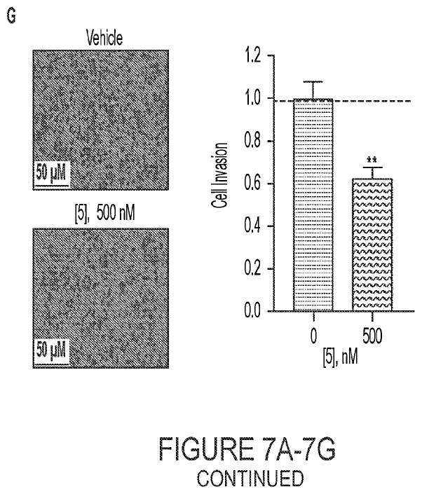

Compound 5 was delivered to MDA-MB-231 cells to assess its ability to cleave both pri-miR-17-92 and its cognate pre-miRNAs. Notably, cellular localization studies showed that 5 can be found throughout the cells, with enhanced fluorescence observed in the perinuclear space (FIG. 14). Indeed, 5 cleaved pri-miR-17-92, reducing its levels by 33(±8)% at 100 nM, with control compound 6 having no effect (FIG. 7A). A competitive cleavage experiment between 2 and 5 was investigated. If the two compounds bind the same site(s) within pri-miR-17-92 then increasing concentration of 2 should diminish cleavage in this competition experiment. Indeed, 2 restored pri-miR-17-92 levels in a dose-dependent fashion (FIG. 7B). Mirroring these results, 5 (500 nM) diminished pre-miR-17 levels by 33(±7)%, pre-miR-20a by 46(±6)% (FIG. 7C), and all six mature miRNAs from 40(±7) to 25(±15)% (FIG. 7D). We corroborated our relative analysis by conducting absolute quantification of mature, pre- and pri-miRNA levels (FIG. 19B).

Reduction of miR-17-92 levels de-repressed both ZBTB4 mRNA and protein, by 36(±16)% and 62(±11)%, respectively, (500 nM of 5; FIGS. 7E and 7F). Further, 5 diminished the invasive characteristics of MDA-MB-231 cells (FIG. 7G). As described for 2, studies were completed for investigation of whether 5 indeed acts along the miR-17-92-ZBTB4 axis by overexpressing pri-miR-17-92 or ablating ZBTB4 with an shRNA. Indeed, both types of experiments ablated 5's ability to de-repress ZBTB4 mRNA and its anti-invasive phenotype (FIG. 19C-19G).

To assess 5's selectivity, miRNAs were identified that contain the same motifs as those targeted by 2 in the miR-17-92 cluster and those predicted by Inforna to bind 2 [dubbed RNA isoforms (n=30); Table 2 with identifier SEQ ID NO:'s]. All 30 miRNAs only contain a single binding site for 1, four of which are located in a Dicer site and one in a Drosha site (Table 2). Treatment of 5, did not affect the levels of any of these miRNA isoforms, underscoring 5's selectivity for the cluster (FIG. 19H). The well-known oncogenic miR-21⁵¹ contains an A bulge in its Dicer site and a U bulge four base pairs downstream; three base pairs separate the bulges in pre-miR-18a and they are in different orientations (same side of the helix in pre-miR-21 and opposite sides in pre-miR-18a (Table 2). Further the A and U bulges in miR-21 have different closing base pairs than pre-miR-18a and are predicted by Inforna to bind 1 weakly. Indeed, 5 had no effect on mature miR-21 levels (FIG. 19H). Collectively, these data support that: (i) 5 directly cleaved the pri-miR-17-92 transcript and induced an anti-invasive phenotype that is dependent on the downregulation of the cluster via ZBTB4 de-repression; and (ii) its selectivity is due to precise display of the RNA-binding modules at a distance that mimics the distance between the bulges, three base pairs.

TABLE 1
Summary of Oligonucleotides used

| | SEQ ID | | | |
|---|---|---|---|---|
| Oligonucleotide | NO: | Sequence 5′ → 3′ | Experiment | Supplier |
| hsa-miR-17 | 4 | CAAAGTGCTTACAGTGCAGGTAC | RT-qPCR | Europhins |
| has-miR-18a | 5 | TAAGGTGCATCTAGTGCAGATAG | RT-qPCR | Europhins |
| has-miR-19a | 6 | TGTGCAAATCTATGCAAAACTGA | RT-qPCR | Europhins |
| has-miR-20a | 7 | TAAAGTGCTTATAGTGCAGGTAG | RT-qPCR | Europhins |
| has-miR-19b | 8 | TGTGCAAATCCATGCAAAACTGA | RT-qPCR | Europhins |
| has-miR-92a- | 9 | TATTGCACTTGTCCCGGCCTGT | RT-qPCR | Europhins |
| 1 | | | | |
| RNU6 | 10 | ACACGCAAATTCGTGAAGCGTTC | RT-qPCR | IDT |
| Universal | 11 | GAATCGAGCACCAGTTACGC | RT-qPCR | IDT |
| Reverse | | | | |
| Pri-miR- | 12 | GGAATTAATTGCTGTTAGGAGGTTGGA | RT-qPCR | IDT |
| 17/92 5′ Fwd. | | | | |
| Pri-miR- | 13 | AGGTCCACGTGTATGACTGG | RT-qPCR | IDT |
| 17/92 5′ Rev | | | | |
| Pri-miR- | 14 | TTATGTTCCCTACTCCCTACGTAAGC | RT-qPCR | IDT |
| 17/92 3′ Fwd. | | | | |
| Pri-miR- | 15 | AGAAAAGAGAGAAGGCAGAAATGCTG | RT-qPCR | IDT |
| 17/92 3′ Rev | | | | |
| STK4 Fwd. | 16 | GATGGGCACTGTCCGAGTAG | RT-qPCR | IDT |
| STK4 Rev | 17 | GCAACGTGTCATCGTGCTC | RT-qPCR | IDT |
| ZBTB4 Fwd. | 18 | GGCACGAACTGACAAGACTTGA | RT-qPCR | IDT |
| ZBTB4 Rev | 19 | TGTGGCGACGTGATTAA | RT-qPCR | IDT |
| 18S Fwd. | 20 | GTAACCCGTTGAACCCCATT | RT-qPCR | IDT |
| 18S Rev | 21 | CCATCCAATCGGTAGTAGCG | RT-qPCR | IDT |
| GAPDH Fwd. | 22 | GTTCGACAGTCAGCCGCATC | RT-qPCR | IDT |
| GAPDH Rev | 23 | GGAATTTGCCATGGGTGGA | RT-qPCR | IDT |
| miR-17 Dicer | 24 | GUGCAGGUAGUGAUAUGUGCAUCUACUGC | Binding Assay | Dharmacon |
| Site Mimic | | AC | | |
| miR-17 Dicer | 25 | TAATACGACTCACTATAGG | Binding Assay | IDT |
| Site Mimic | | | | |
| Mutant Fwd. | | | | |
| miR-17 Dicer | 26 | TAATACGACTCACTATAGGGTGCAGGTAGA | Binding Assay | IDT |
| Site G Bulge | | TGATATGTGCATCTACTGCAC | | |
| T7 template | | | | |
| miR-17 Dicer | 27 | GTGCAGTAGATGCACATA | Binding Assay | IDT |
| Site G Bulge | | | | |
| Rev | | | | |
| miR-17 Dicer | 28 | TAATACGACTCACTATAGGGTGCAGGTAGT | Binding Assay | IDT |
| Site U Bulge | | GATATGTGCATCTACCTGCAC | | |
| Mimic | | | | |
| miR-17 Dicer | 29 | GTGCAGGTAGATGCACAT | Binding Assay | IDT |
| Site G Bulge | | | | |
| Rev | | | | |
| miR-17 Base | 30 | GUGCAGGUAGAUGAUAUGUGCAUCUACCU | Binding Assay | Dharmacon |
| Pair Control | | GCAC | | |
| Pre-miR-17 | 31 | GGCCGGATCCTAATACGACTCACT | Dicer Inhibition | IDT |
| T7 Fwd. | | ATAGGTCAAAGTGCTTACAGTGCAGG | | |
| Pre-miR-17 | 32 | GCTACAAGTGCCTTCACTG | Dicer Inhibition | IDT |
| Rev | | | | |
| Pre-miR-17 | 33 | TCAAAGTGCTTACAGTGCAGGTAGTGA | Dicer Inhibition | IDT |
| Template | | TATGTGCATCTACTGCAGTGA AGG CAC | | |
| | | TTG TAGC | | |
| Pre-miR-17- | 34 | TCAAAGTGCTTACAGTGCAGGTAG | Dicer Inhibition | IDT |
| G21 Mutant | | TGATATGTGCATCTACCTGCAGTGA | | |
| Template | | AGGCACTTGTAGC | | |
| Pre-miR-17- | 35 | TCAAAGTGCTTACAGTGCAGGTAGA | Dicer Inhibition | IDT |
| U37 Mutant | | TGATATGTGCATCTACTGCAGTGAAGG | | |
| Template | | CACTTGTAGC | | |
| Pre-miR17- | 36 | TCAAAGTGCTTACAGTGCAGGTAG | Dicer Inhibition | IDT |
| G21/U37 | | ATGATATGTGCATCTACCTGCAGT | | |
| Mutant | | GAAGGCACTTG | | |
| Template | | | | |
| Pre-miR-18a | 37 | GGCCGCATGGTAATACGACTCACTATAGGT | Dicer Inhibition | IDT |
| T7 Fwd | | AAGGTGCAT CTAGTGCAG | | |
| Pre-miR-18a | 38 | CCAGAAGGAGCACTTAGG | Dicer Inhibition | IDT |
| Rev | | | | |
| Pre-miR-18a | 39 | TAAGGTGCATCTAGTGCAGATAGTGAAGTA | Dicer Inhibition | IDT |
| Template | | GATTAG | | |
| | | CATCTACTGCCCTAAGTGCTCCTTCTGG | | |
| Pre-miR-18a- | 40 | TAAGGTGCATCTAGTGCAGATAGATGAAGT | Dicer Inhibition | IDT |
| U37 | | AGATTAG | | |
| Template | | CATCTACTGCCCTAAGTGCTCCTTCTGG | | |
| Pre-miR-20a | 41 | GGCCGGATCCTAATACGACTCACTATAGGG | Dicer Inhibition | IDT |
| T7 Fwd | | ACTAAAGTGCTTATAGTGCAGG | | |
| Pre-miR-20a | 42 | ACTTTAAGTGCTCATAATGCAG | Dicer Inhibition | IDT |
| Rev | | | | |
| Pre-miR-20a | 43 | ACTAAAGTGCTTATAGTGCAGGTAGTGTTTA | Dicer Inhibition | IDT |
| Template | | GTTATCTACTGCATTATGAGCACTTAAAGT | | |
| Pre-miR-19a | 44 | GGCCGGATCCTAATACGACTCACTATAGGG | Dicer Inhibition | IDT |
| T7 Fwd | | TTAGTTTTGCATAGTTGCACT | | |
| Pre-miR-19a | 45 | TCAGTTTTGCATAGATTTGCA | Dicer Inhibition | IDT |
| Rev | | | | |
| Pre-miR-19a | 46 | TTAGTTTTGCATAGTTGCACTACAAGAAGA | Dicer Inhibition | IDT |
| Template | | ATGTAGTTGTGCAAATCTATGCAAAACTGA | | |
| Pre-miR-19b | 47 | GGCCGGATCCTAATACGACTCACTATAGGG | Dicer Inhibition | IDT |
| T7 Fwd | | TTAGTTTTGCAGGTTTGCA | | |
| Pre-miR-19b | 48 | AGTCAGTTTTGCATGGATTTG | Dicer Inhibition | IDT |
| Rev | | | | |
| Pre-miR-19b | 49 | GGTTAGTTTTGCAGGTTTGCATCCAGCTGTG | Dicer Inhibition | IDT |
| Template | | TGATATTC | | |
| | | TGCTGTGCAAATCCATGCAAAACTGACT | | |
| Pre-miR-92a- | 50 | GGCCGGATCCTAATACGACTCACTATAGGG | Dicer Inhibition | IDT |
| 1 Fwd. | | CACAGGTTGGGATCGGTT | | |
| Pre-miR-92a- | 51 | AACAGGCCGGGACAAGT | Dicer Inhibition | IDT |
| 1 Rev | | | | |
| Pre-miR-92a- | 52 | CACAGGTTGGGATCGGTTGCAATGCTGTGTT | Dicer Inhibition | IDT |
| 1 Template | | TCTGTATGGTATTGCACTTGTCCCGGCCTGT | | |
| | | T | | |
| PD-L1 | 53 | TGGACAAGCAGTGACCATCAA | RT-qPCR | IDT |
| Forward | | | | |
| PD-L1 | 54 | GGATGTGCCAGAGGTAGTTC | RT-qPCR | IDT |
| Reverse | | | | |
| Pre-miR-18a | 55 | TAAGGTGCATCTAGTGCAGATAG | RT-qPCR | IDT |
| Fwd. | | | | |
| Pre-miR-18a | 56 | GAAGGAGCACTTAGGGCAGT | RT-qPCR | IDT |
| Rev | | | | |
| T7-miR-17 | 57 | TAATACGACTCACTATAGGCAAAGTGCTTA | Transcription | IDT |
| template | | CAGTGCAGGTAG | | |
| T7-miR-18a | 58 | TAATACGACTCACTATAGGTAAGGTGCATC | Transcription | IDT |
| template | | TAGTGCAGATAG | | |
| T7-miR-20a | 59 | TAATACGACTCACTATAGGTAAAGTGCTTAT | Transcription | IDT |
| template | | AGTGCAGGTAG | | |
| T7-miR-19a | 60 | TAATACGACTCACTATAGGTGTGCAAATCT | Transcription | IDT |
| template | | ATGCAAAACTGA | | |
| T7-miR-19b- | 61 | TAATACGACTCACTATAGGTGTGCAAATCC | Transcription | IDT |
| l template | | ATGCAAAACTGA | | |
| T7-miR-92a- | 62 | TAATACGACTCACTATAGGTATTGCACTTGT | Transcription | IDT |
| l template | | CCCGGCCTGT | | |
| Pri-miR-17- | 63 | TAATACGACTCACTATAGGAATTAATTGCTG | Transcription | IDT |
| 92 5′ Fwd | | TTAGGAGGTTGGAAAATAGCAAATATAG | | |
| Pri-miR-17- | 64 | TTAGGAGGTTGGAAAATAGCAAATATAGAT | Transcription | IDT |
| 92 5′ | | TTGGACGGTGGTAGTAATTTTGAGCAAATA | | |
| Template | | ATGTTTTATCTTTTTTTTCCTTAT | | |
| Pri-miR-17- | 65 | AGGTCCACGTGTATGACTGGAATAGGGAAA | Transcription | IDT |
| 92 5′ Rev | | AATAAGGAAAAAAAAGATAAAACATTAT | | |

TABLE 2
Secondary structures of RNA isoforms and the miR-21 hairpin precursor

| | SEQ | | |
|---|---|---|---|
| Mo- | ID | | |
| tif | NO | miRNA | Secondary Structure |
| 5′ GAU/ 3′ C_A | 66 | hsa- miR- 363 | 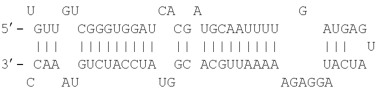 |
| | 67 | hsa- miR- 3945 | 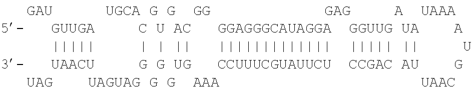 |
| | 68 | hsa- miR- 4435- 1 | 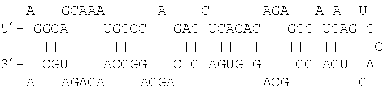 |
| | 69 | hsa- miR- 196a- 2 | 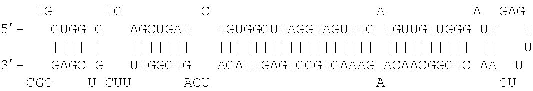 |
| | 70 | hsa- miR- 3168 | 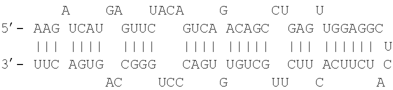 |
| | 71 | hsa- miR- 4640 | 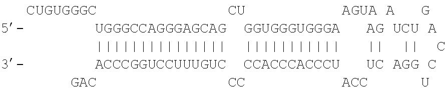 |
| | 72 | hsa- miR- 101- 1 | 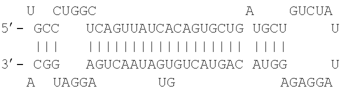 |
| | 73 | hsa- miR- 4700 | 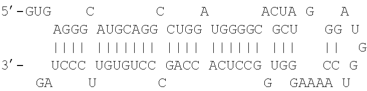 |
| | 74 | hsa- miR- 1226 | 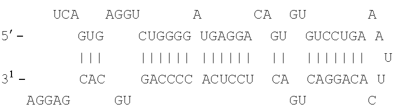 |
| | 75 | hsa- miR- 155 | 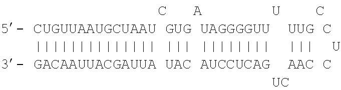 |
| | 76 | hsa- miR- 4273 | 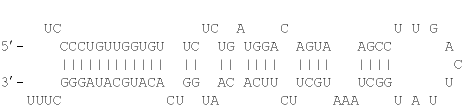 |
| | 77 | hsa- miR- 4454 | 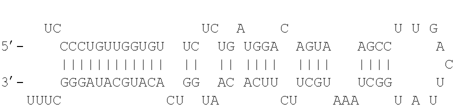 |
| | 78 | hsa- miR- 4435- 2 | 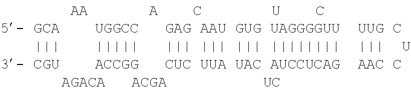 |
| | 79 | hsa- let- 7g | 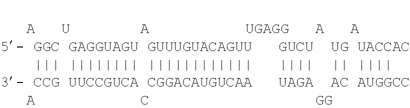 |
| 5′ G_ U/3′ CUA | 80 | hsa- miR- 539 | UG A G UUU 5′- AUACU AGGAGAAAUU UCCUUG UGUG UUCG C A \|\|\|\|\| \|\|\|\|\|\|\|\|\|\| \|\|\|\|\|\| \|\|\|\| \|\|\|\| \| U 3′- UAUGA UUUUCUUUAA AGGAAC AUAC AAGU G A GU C U A UAU |
| | 81 | hsa- miR- 4267 | 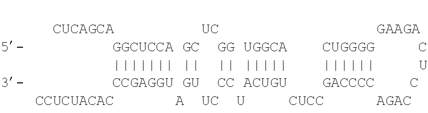 |
| | 82 | hsa- miR- 571 | 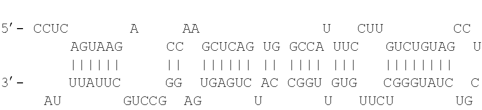 |
| | 83 | hsa- miR- 153- 1 | 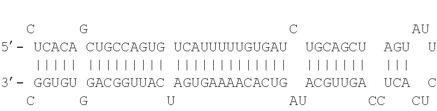 |
| | 84 | hsa- miR- 153- 2 | 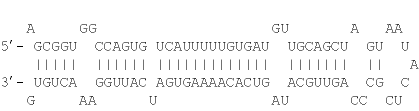 |
| | 85 | hsa- miR- 222 | 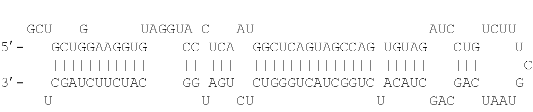 |
| | 86 | hsa- miR- 3180- 4 | 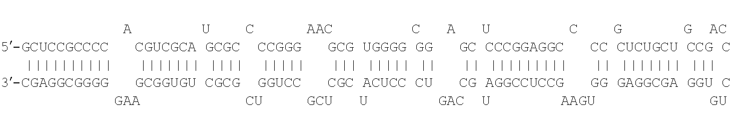 |
| | 87 | hsa- miR- 19b- 2 | 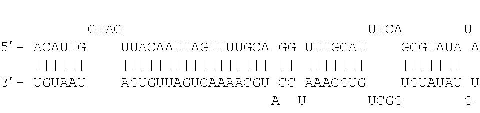 |
| | 88 | hsa- miR- 487a | 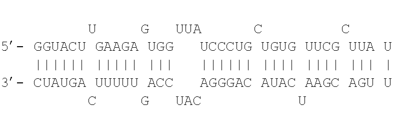 |
| 5′ GGU/ 3′ C_A | 89 | hsa- miR- 658 | 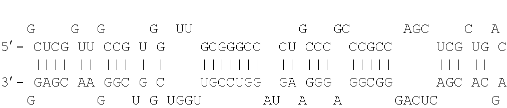 |
| | 90 | hsa- miR- 1197 | 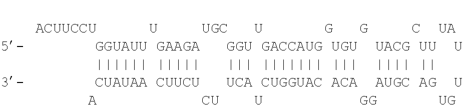 |
| | 91 | hsa- miR- 662 | 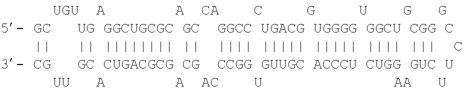 |
| | 92 | hsa- miR- 93 | 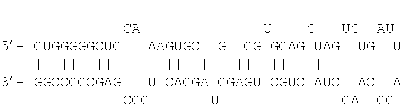 |
| | 93 | hsa- miR- 548a- 2 | 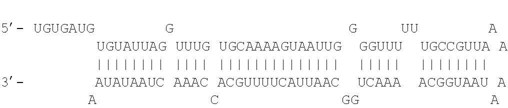 |
| | 94 | hsa- miR- 27b | 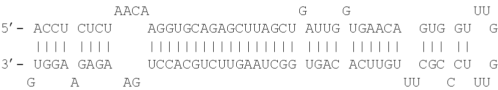 |
| | 95 | hsa- miR- 3178 | 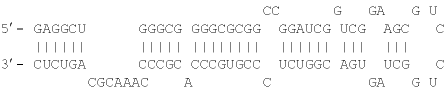 |
| | 96 | hsa- miR- 1324 | 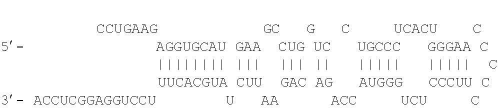 |
| | 97 | hsa- miR- 106a | 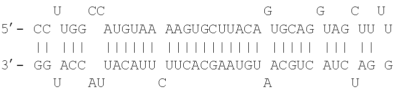 |
| | 98 | hsa- miR- 21 | A A A U AA 5′-UGUCGGGUAGCUUAUC GACUG UGUUG CUGU G U 3′-ACAGUCUGUCGGGUAG-CUGAC ACAAC-GGUA-C C UC |

### Cleavage of the Pri-miR-17-92 Cluster by 5 in DU-145 Prostate Cancer Cells.

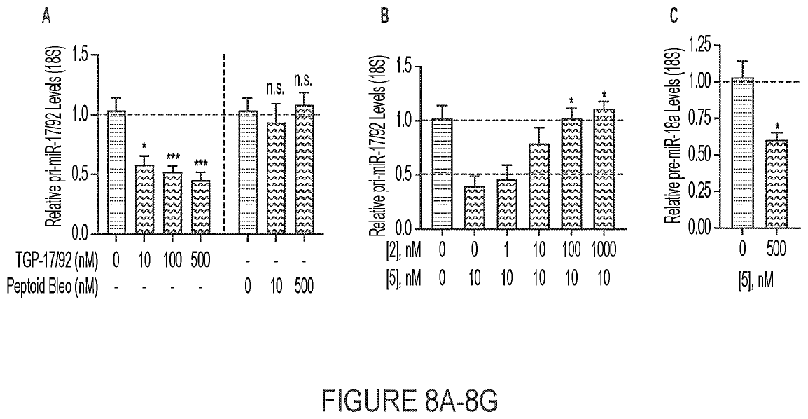

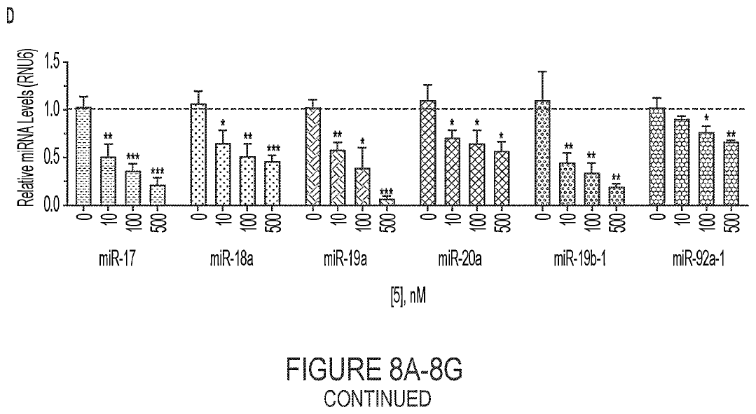

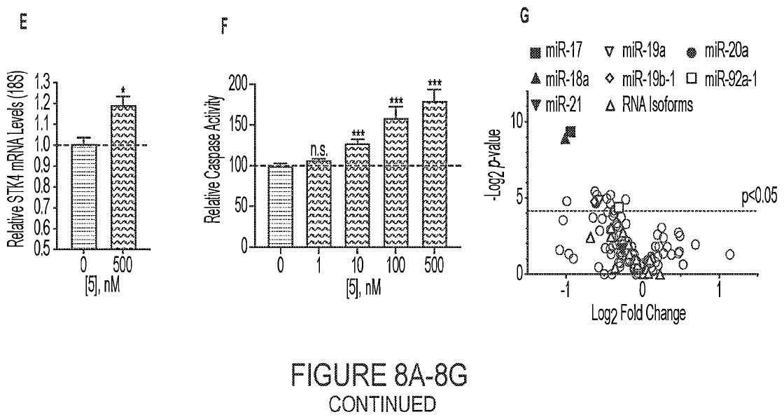

Treatment of DU-145 cells with 5 decreased the levels of pri-miR-17-92 dose-dependently, with significant cleavage observed with as little as 10 nM of compound. No change in pri-miR-17-92 levels were observed with control compound 6 up to the highest concentration tested (500 nM), indicating selective cleavage of the cluster by 5 (FIG. 8A). Competitive cleavage studies (completed at a constant concentration of 10 nM of 5, the approximate IC₅₀) showed that 2 restored pri-miR-17-92 levels in a dose dependent fashion, validating that they both bind the same site(s) within the target (FIG. 8B). Furthermore, 5 also diminished pre-miR-18a levels by 40(±4)% at 100 nM (FIG. 8C), and levels of all miRNAs within the cluster with treatment with as little as 10 nM compound, except for miR-92a-1 where inhibitory effects were significant at 100 and 500 nM concentrations (FIG. 8D). In agreement with studies of the pri-miRNA, 6 did not affect levels of mature miRNAs within the cluster except miR-19a, which decreased by ˜50% at 500 nM (FIG. 20A), consistent with results obtained from absolute quantification (FIG. 20B). This is likely due to the stretches of AU pairs in its hairpin precursor, which were shown previously to be a target of bleomycin A5 itself.⁵⁰

The effect of the 5 on the levels of miR-18a's downstream target (STK4) in DU-145 cells and on phenotype was also assessed. Indeed, application of 5 (500 nM) increased levels of Stk4 mRNA by 19(±4)% (FIG. 8E) and protein levels by 2.6-fold (FIG. 20C). Further, the compound triggered apoptosis in DU-145 cells, as measured via induction of Caspase 3/7, with statistically significant effects observed with as little as 10 nM compound (FIG. 8F), consistent with its effect on pri-miR-17-92 levels (FIG. 8A). Control compound 6 did not induce apoptosis, as expected (FIG. 20D). By comparing the IC₅₀'s of 2 and 5 for knockdown of mature miR-18a or induction of apoptosis, enabling small molecules to cleave the desired target increased potency by ˜10-fold (FIGS. 4A vs. 8D and 4E vs. 8F).

Similar to the studies completed for 2 (FIG. 17C), a study was conducted to determine whether 5's rescue of phenotype is due to inhibiting the miR-18a-STK4 circuit via cleavage of the primary transcript. Indeed, overexpression of the cluster from a plasmid attenuated 5's activity, as assessed by cleavage of pri-miR-17-92, de-repression of STK4 mRNA, and activation of Caspase 3/7 (FIG. 20E-20G). Interestingly, while the STK4 shRNA had no effect on 5's ability to cleave pri-miR-17-92, it ablated de-repression of Stk4 mRNA and activation of Caspase 3/7 (FIG. 20H-20J). Taken together, these data support that 5's activity is a direct result of downregulating the pri-miR-17-92 cluster and that induction of apoptosis is dependent on de-repression of STK4.

### Selectivity of 5 in DU-145 Cells.

To assess the selectivity of 5 cleavage on the miRNome, the levels of all expressed miRNAs in DU-145 cells (n=373) were measured upon treatment with 500 nM of 5 by RT-qPCR. As shown in FIGS. 8G and S11K, the most affected miRNAs were those derived from the cluster, with no statistically significant effects on non-cluster miRNAs, RNA isoforms, or miR-21. As mentioned above, none of these RNAs has two bulges separated by the same distance as in pre-miR-17, -18a, or -20a. Collectively, these studies suggest that 5 is a selective cleaver of the miR-17-92 cluster amongst the miRNome.

Next, it was assessed whether 5 has selective effects on the proteome. Global proteomics analysis showed that only 9/3730 (0.24%) proteins were significantly affected by 5 treatment (FIG. 21A), and only four were correlated with changes in mRNA levels (FIG. 21B). Among downregulated proteins, zinc finger CCCH domain-containing protein 7A (ZC3H7A) was found to be abnormally expressed in pancreatic ductal adenocarcinoma (PDAC) and mutation of ZC3H7A is related to metastasis of PDAC.⁵² Interestingly, programmed cell death 1 ligand 1 (PDL1), a known to be a target of miR-17,^{53,54} was upregulated, although its cell surface expression was not affected, as assessed by immunohistochemical staining and flow cytometry (FIG. 21C-21E). The BRI3 binding protein (BRI3BP), a pro-apoptotic protein that increases drug-induced apoptosis by increasing cytochrome c release and improving Caspase-3 activity,^{55,56} was also upregulated. Please see the experimental section for a detailed discussion of this proteomics analysis (FIGS. 21A-21E).

Design and Evaluation of a Pri-miR-17-92 Cluster and Pre-miR-17-, miR-18a-, and miR-20a-Targeting RIBOTAC.

An alternative strategy was developed to target members of the miR-17-92 cluster for destruction by recruiting an endogenous nuclease via a ribonuclease targeting chimera (RIBOTAC).^{3,57,58} A RIBOTAC comprises an RNA-binding small molecule, in this case 2, conjugated to an RNase L-recruiting module, in this case a recently discovered heterocycle, affording RIBOTAC 7 (FIG. 9A).⁵⁷ RNase L functions in innate immunity and is expressed at minute levels in all cells as an inactive monomer. It is activated and dimerized upon viral infection and has inherent substrate specificity, preferring 5′UA and 5′UU steps.^{59,60} RIBOTACs locally recruit RNase L to the desired target to effect selective cleavage.^{3,57,58}

Therefore, a study was conducted to determine whether the pri-miRNA, pre-miRNAs, or both are targeted for degradation by RIBOTAC 7. Interestingly, fluorescence microscopy showed that 7 was dispersed throughout the cell, but notably in the cytoplasm where the pre-miRNA targets and RNase L also reside (FIG. 14). These results suggest that 7 could trigger cleavage of pre-miR-17, -18a, and 20a, which bind 2, but not the cleavage of pri-miR-17-92 as RNase L is not detectable in the nucleus. That is, cleavage only occurs in the cytoplasm where all three components of the ternary complex are present, i.e., 7, its RNA targets, and RNase L. Notably, 7 was taken up to a similar extent as 2 in both DU-145 and MDA-MB-231 cells (FIG. 9B).

To study if this is indeed the case, the levels were measured for mature miRNAs, pre-miRNAs, and the primary transcript of the 17/92 cluster in MDA-MB-231 TNBC cells upon treatment with 7. Indeed, 7 reduced the levels of mature miR-17, miR-18a, and miR-20a dose dependently, with no effect on the levels of the other three mature miRNAs in the cluster (FIG. 9C). Consistent with the hypothesis, 7 (500 nM) decreased levels of pre-miR-17, miR-18a, and miR-20a by 31(±3)%, 19(±4)%, and 36(±3)%, respectively (FIG. 9D), while pri-miR-17/92 levels were unchanged (FIG. 9E). To corroborate these data, the effect of 7 on levels of the cluster, whether mature, pre-, or pri-miRNAs, was also measured in DU-145 cells. The same trends were observed; that is, only the pre-miRNAs that bind 2 were degraded, as evidenced by reduced levels of their mature (FIG. 9F) and pre-miRNAs (FIG. 9D). (No effect on pri-miR-17/92 levels was observed (FIG. 9E).) The changes observed in both cell lines were verified by absolute quantification (FIGS. 22A-22B).

### Mechanism of Action and Medical Treatment

In certain embodiments, the invention is directed to methods of inhibiting, suppressing, derepressing and/or managing biolevels of the miRNA-X's, pre-miRNA-X's (X being a designator for group of specific numbers of the miRNA's encompassed according to the invention such as miR-17 and miR-20a), and/or the corresponding pri-miR-17-92 cluster, pre-miR-17, pre-miR-18a and/or pre-miR-20a and/or any mixture thereof as well as these RNA entities present in oncologic cell lines and in animals and humans having such oncologic cells and present in polycystic cell lines and in animals and humans have polycystic disease. The Compounds 1, 1D, 2, 5, and/or 7 as embodiments of the invention for use in the methods disclosed herein bind to the above identified RNA entities as well in the above identified cell lines, animals and humans.

Embodiments of the Compounds applied in methods of the invention and their pharmaceutical compositions are capable of acting as “inhibitors”, suppressors and or modulators of the above identified RNA entities which means that they are capable of blocking, suppressing or reducing the expression of the RNA entities. An inhibitor can act with competitive, uncompetitive, or noncompetitive inhibition. An inhibitor can bind reversibly or irreversibly.

The compounds useful for methods of the invention and their pharmaceutical compositions function as therapeutic agents in that they are capable of preventing, ameliorating, modifying and/or affecting a disorder or condition. The characterization of such compounds as therapeutic agents means that, in a statistical sample, the compounds reduce the occurrence of the disorder or condition in the treated sample relative to an untreated control sample, or delays the onset or reduces the severity of one or more symptoms of the disorder or condition relative to the untreated control sample.

The ability to prevent, ameliorate, modify and/or affect in relation to a condition, such as a local recurrence (e.g., pain), a disease known as a polycystic disease including but not limited to polycystic kidney disease or an oncologic disease such as but not limited to breast cancer and/or prostate cancer or any other neoplastic and/or oncologic disease or condition, especially having etiology similar to breast and/or prostate cancer may be accomplished according to the embodiments of the methods of the invention and includes administration of a composition as described above which reduces, or delays or inhibits or retards the oncologic medical condition in a subject relative to a subject which does not receive the composition.

The compounds of the invention and their pharmaceutical compositions are capable of functioning prophylactically and/or therapeutically and include administration to the host/patient of one or more of the subject compositions. If it is administered prior to clinical manifestation of the unwanted condition (e.g., disease or other unwanted state of the host animal/patient) then the treatment is prophylactic, (i.e., it protects the host against developing the unwanted condition), whereas if it is administered after manifestation of the unwanted condition, the treatment is therapeutic, (i.e. it is intended to diminish, ameliorate, or stabilize the existing unwanted condition or side effects thereof).

The compounds of the invention and their pharmaceutical compositions are capable of prophylactic and/or therapeutic treatments. If a compound or pharmaceutical composition is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof). As used herein, the term “treating” or “treatment” includes reversing, reducing, or arresting the symptoms, clinical signs, and underlying pathology of a condition in manner to improve or stabilize a subject's condition.

The compounds of the invention and their pharmaceutical compositions can be administered in “therapeutically effective amounts” with respect to the subject method of treatment. The therapeutically effective amount is an amount of the compound(s) in a pharmaceutical composition which, when administered as part of a desired dosage regimen (to a mammal, preferably a human) alleviates a symptom, ameliorates a condition, or slows the onset of disease conditions according to clinically acceptable standards for the disorder or condition to be treated, e.g., at a reasonable benefit/risk ratio applicable to any medical treatment.

### Administration

Compounds of the invention and their pharmaceutical compositions prepared as described herein can be administered according to the methods described herein through use of various forms, depending on the disorder to be treated and the age, condition, and body weight of the patient, as is well known in the art. As is consistent, recommended and required by medical authorities and the governmental registration authority for pharmaceuticals, administration is ultimately provided under the guidance and prescription of an attending physician whose wisdom, experience and knowledge control patient treatment.

For example, where the compounds are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route or other similar transmucosal route, they may be formulated as drops or ointments.

These formulations for administration orally or by a transmucosal route can be prepared by conventional means, and if desired, the active ingredient may be mixed with any conventional additive or excipient, such as a binder, a disintegrating agent, a lubricant, a corrigent, a solubilizing agent, a suspension aid, an emulsifying agent, a coating agent, a cyclodextrin, and/or a buffer. Although the dosage will vary depending on the symptoms, age and body weight of the patient, the gender of the patient, the nature and severity of the disorder to be treated or prevented, the route of administration and the form of the drug, in general, a daily dosage of from 0.0001 to 2000 mg, preferably 0.001 to 1000 mg, more preferably 0.001 to 500 mg, especially more preferably 0.001 to 250 mg, most preferably 0.001 to 150 mg of the compound is recommended for an adult human patient, and this may be administered in a single dose or in divided doses. Alternatively, a daily dose can be given according to body weight such as 1 nanogram/kg (ng/kg) to 200 mg/kg, preferably 10 ng/kg to 100 mg/kg, more preferably 10 ng/kg to 10 mg/kg, most preferably 10 ng/kg to 1 mg/kg. The amount of active ingredient which can be combined with a carrier material to produce a single dosage form will generally be that amount of the compound which produces a therapeutic effect.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

The phrase “pharmaceutically acceptable” is employed herein to refer to those excipients, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### Pharmaceutical Compositions Incorporating Compounds 1, 1D, 2, 4FL, 5 and 7

The pharmaceutical compositions of the invention incorporate embodiments of Compounds 1, 1D, 2, 5 and/or 7 useful for methods of the invention and a pharmaceutically acceptable carrier. The compositions and their pharmaceutical compositions can be administered orally, topically, parenterally, by inhalation or spray or rectally in dosage unit formulations. The term parenteral is described in detail below. The nature of the pharmaceutical carrier and the dose of these Compounds depend upon the route of administration chosen, the effective dose for such a route and the wisdom and experience of the attending physician.

A “pharmaceutically acceptable carrier” is a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted (3-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of a compound of the invention as an active ingredient. A composition may also be administered as a bolus, electuary, or paste.

In solid dosage form for oral administration (capsules, tablets, pills, dragees, powders, granules, and the like), a compound of the invention is mixed with one or more pharmaceutically acceptable carriers, such as sodium citrate or dicalcium phosphate, and/or any of the following:

(1) fillers or extenders, such as starches, cyclodextrins, lactose, sucrose, glucose, mannitol, and/or silicic acid;

(2) binders, such as, for example, carboxymethylcellulose, alginates, gelatin, polyvinyl pyrrolidone, sucrose, and/or acacia;

(3) humectants, such as glycerol;

(4) disintegrating agents, such as agar-agar, calcium carbonate, potato or tapioca starch, alginic acid, certain silicates, and sodium carbonate;

(5) solution retarding agents, such as paraffin;

(6) absorption accelerators, such as quaternary ammonium compounds;

(7) wetting agents, such as, for example, acetyl alcohol and glycerol monostearate;

(8) absorbents, such as kaolin and bentonite clay;

(9) lubricants, such a talc, calcium stearate, magnesium stearate, solid polyethylene glycols, sodium lauryl sulfate, and mixtures thereof; and

(10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor(s) moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. A compound of the invention can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more inhibitor(s) with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the active agent.

Formulations which are suitable for vaginal administration also include pessaries, tampons, creams, gels, pastes, foams, or spray formulations containing such carriers as are known in the art to be appropriate.

Dosage forms for the topical or transdermal administration of an inhibitor(s) include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to a compound of the invention, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound of the invention, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

A compound useful for application of methods of the invention can be alternatively administered by aerosol. This is accomplished by preparing an aqueous aerosol, liposomal preparation, or solid particles containing the composition. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of a compound of the invention together with conventional pharmaceutically acceptable carriers and stabilizers. The carriers and stabilizers vary with the requirements of the particular composition, but typically include nonionic surfactants (Tweens, Pluronics, sorbitan esters, lecithin, Cremophors), pharmaceutically acceptable co-solvents such as polyethylene glycol, innocuous proteins like serum albumin, oleic acid, amino acids such as glycine, buffers, salts, sugars, or sugar alcohols. Aerosols generally are prepared from isotonic solutions.

Transdermal patches have the added advantage of providing controlled delivery of a compound of the invention to the body. Such dosage forms can be made by dissolving or dispersing the agent in the proper medium. Absorption enhancers can also be used to increase the flux of the inhibitor(s) across the skin. The rate of such flux can be controlled by either providing a rate controlling membrane or dispersing the inhibitor(s) in a polymer matrix or gel.

Pharmaceutical compositions of this invention suitable for parenteral administration comprise one or more compounds of the invention in combination with one or more pharmaceutically acceptable sterile aqueous or nonaqueous solutions, dispersions, suspensions or emulsions, or sterile powders which may be reconstituted into sterile injectable solutions or dispersions just prior to isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a compound useful for practice of methods of the invention, it is desirable to slow the absorption of the compound from subcutaneous or intramuscular injection. For example, delayed absorption of a parenterally administered drug form is accomplished by dissolving or suspending the drug in an oil vehicle. Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The pharmaceutical compositions may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The pharmaceutical compositions of the invention may be “systemically administered” “administered systemically,” “peripherally administered” and “administered peripherally” meaning the administration of a ligand, drug, or other material other than directly into the central nervous system, such that it enters the patient's system and thus, is subject to metabolism and other like processes, for example, subcutaneous administration.

The compound(s) useful for application of the methods of the invention may be administered to humans and other animals for therapy by any suitable route of administration, including orally, nasally, as by, for example, a spray, rectally, intravaginally, parenterally, intracisternally, and topically, as by powders, ointments or drops, including buccally and sublingually.

Regardless of the route of administration selected, the compound(s) useful for application of methods of the invention, which may be used in a suitable hydrated form, and/or the pharmaceutical compositions of the present invention, are formulated into pharmaceutically acceptable dosage forms by conventional methods known to those of skill in the art.

Actual dosage levels of the compound(s) useful for application of methods of the invention in the pharmaceutical compositions of this invention may be varied so as to obtain an amount of the active ingredient which is effective to achieve the desired therapeutic response for a particular patient, composition, and mode of administration, without being toxic to the patient.

The concentration of a compound useful for application of methods of the invention in a pharmaceutically acceptable mixture will vary depending on several factors, including the dosage of the compound to be administered, the pharmacokinetic characteristics of the compound(s) employed, and the route of administration.

In general, the compositions useful for application of methods of this invention may be provided in an aqueous solution containing about 0.1-10% w/v of a compound disclosed herein, among other substances, for parenteral administration. Typical dose ranges are those given above and may preferably be from about 0.001 to about 500 mg/kg of body weight per day, given in 1-4 divided doses. Each divided dose may contain the same or different compounds of the invention. The dosage will be an effective amount depending on several factors including the overall health of a patient, and the formulation and route of administration of the selected compound(s).

### EXPERIMENTAL SECTION

General Methods. Synthetic RNAs were obtained from Dharmacon. They were deprotected according to the manufacturers protocol and de-salted using PD-10 sephadex columns (GE Healthcare) per the manufacturers protocol. DNA templates and primers were obtained from IDT and used without further purification. Locked Nucleic Acid inhibitors were purchased from Exiqon and resuspended in TE buffer directly. HEK 293T, MDA-MB-231 and DU145 cells were obtained from ATCC and used directly. HEK-293T and WT-9-12 cells were maintained in 1×DMEM (Corning) supplemented with 1×glutaGRO (Corning-) Penicillin/Streptomycin (50 U/mL) and 10% (v/v) fetal bovine serum (Sigma) [growth medium]. Proper adherence of WT-9-12 cells required coating of dishes with PurCol Bovine collagen 3 mg/mL (Corning) at 37° C. for 30 min before seeding cells. MDA-MB-23 and DU145 cells were maintained in 1×RPMI 1640 (Corning) supplemented with Penicillin/Streptomycin (50 U/mL) and 10% Fetal Bovine Serum (Sigma) [growth medium]. All cells were grown at 37° C. with 5% CO₂. Chemicals were purchased from the following commercial sources: Combi blocks, Advanced Chem Tech, and Alfa Aesar.

Luciferase assays. HEK 293T cells were plated in six-well dishes (2×10⁵ cells per well) and co-transfected with 0.4 mg of pLS-Renilla-30-UTR plasmids and with 0.04 mg of the pGL3-Control plasmid using jetPrime per the manufacture's protocol for 4 h. Then, the cells were trypsinized and plated into 96-well plates (2*10⁴ cells per well) and allowed to adhere for 12 h after which, they were treated with the Dimer library or vehicle (DMSO) for 24 h. After treatment, Firefly and Renilla luciferase activities were measured by using the Dual-Luciferase Reporter Assay System (Promega Corp) according to the manufacturer's directions. Luminescence was measured on a Molecular Devices M5 plate reader with an integration time of 500 ms.

Binding Affinity Measurements. An in-solution fluorescence-based assay was used to determine the binding affinities of the best dimer to miR-17 and -18a by monitoring the change in fluorescence intensity of 4-FL as a function of RNA concentration. Briefly, the RNA of interest was folded in 1× Folding Buffer (8 mM Na₂HPO₄, pH 7.0, 185 mM NaCl, and 1 mM EDTA) at 60° C. for 5 min and then slowly cooled to room temperature. Then, the 4-FL was added into the RNA solution to a final concentration of 100 nM. Serial dilutions were completed using 1× Folding Buffer supplemented with 100 nM 4-FL compound. The solutions were incubated at room temperature for 30 min and then transferred to a black 384-well plate. Fluorescence intensity was measured using a Bio-Tek FLx800 plate reader with an excitation bandpass filter of 485/20 nm and an emission band pass filter of 528/20 nm. The change in fluorescence intensity as a function of the concentration of RNA was fit to equation 1:

I=I₀+0.5Δε{([FL]₀+[RNA]₀+K_{d})−(([FL]₀+[RNA]₀+K_{d})²−4[FL]₀[RNA]₀)^{1/2}} (1)

where I is the observed fluorescence intensity; I₀ is the fluorescence intensity in the absence of RNA; Δε is the difference between the fluorescence intensity in the absence of RNA and in the presence of infinite RNA concentration; [FL]₀ is the concentration of compound; [RNA]₀ is the concentration of the selected RNA; and K_{d} is the dissociation constant. Competitive binding assays were completed by incubating the RNA of interest with 100 nM 4-FL and increasing concentrations of 2. The resulting curves were fit to equation 2:

θ=1/2[C][K_t+K_t/K_d[C_t]+[RNA]+[C]]−{(K_t+K_t/K_d+[C_t]+[RNA]+[C])−4[C][RNA]} (2)

where θ is the percentage of 4-FL bound, [4-FL] is the concentration of 4-FL, Kt is the dissociation constant of RNA and 4-FL, [RNA] is the concentration of RNA, Ct is the concentration of 4-FL, K_{d} is the dissociation constant for 4, and A is a constant.

Dicer Inhibition assay. The RNA was folded in 1× Reaction Buffer (Genlantis) by heating at 60° C. for 5 min and slowly cooling to room temperature. The samples were then supplemented with 1 mM ATP and 2.5 mM MgCl₂. Serially diluted concentrations of 2 were added, and the samples were incubated at room temperature for 15 min. Next, 7 ng/μL of recombinant human Dicer was added followed by incubation at 37° C. overnight. Reactions were stopped by adding the manufacturer's supplied stop solution (Genlantis). A T1 ladder (cleaves G residues) was generated by heating the RNA in 1×RNA Sequencing Buffer (20 mM sodium citrate, pH 5.0, 1 mM EDTA, and 7 M urea) at 55° C. for 10 min followed by slowly cooling to room temperature. RNase T1 was then added to a final concentration of 10 U/μL, and the solution was incubated at room temperature for 20 min. An RNA hydrolysis ladder was generated by incubating RNA in 1×RNA Hydrolysis Buffer (50 mM NaHCO₃, pH 9.4, and 1 mM EDTA) at 95° C. for 5 min the sample was then snap cooled on ice. In all cases, the cleavage products were separated on a 0.7 mm denaturing 15% polyacrylamide gel and imaged using a Bio-Rad PMI phosphorimager.

RT-qPCR in DU145, MDA-MB-231, and WT-9-12 cells. DU145 cells were seeded into 12-well plates at ˜50% confluency (≈200,000 cells/well) and allowed to adhere for 12 h. After adhering, the cells were treated with compounds 2, 5, 6, or 7 (10, 100, and 500 nM) for 24 h. Total RNA was then harvested using a Zymo-Quick RNA Mini prep kit (Zymo Research) with DNase treatment according in the manufacturers protocol. Reverse transcription (RT) for mature miRNAs was done using the miScript II RT kit (Qiagen) with 200 ng of total RNA. To measure precursor and mRNA levels, RT was done using qScript (Quanta Bio) according to the manufacturers protocol on 1000 ng of total RNA. RT-qPCR was carried out on an Applied Biosystems 7900HT cycler under standard conditions (2 step PCR; 60° C. annealing/elongation, 95° C. melt) using the Power Sybr Master Mix (Applied Biosystems). Data were normalized to RNU6 for mature miRNAs and 18S ribosomal RNA for precursor miRNA's and mRNAs, with expression levels calculated using the ΔΔCt method.⁶ Similar to what was done in DU145 cells, MDA-MB-231 and WT-9-12 cells were cultured in 6 well or 12-well plates and treated with compounds 2, 5, 6, or 7 for 24 h. Total RNA was extracted in a similar manner and subjected to RT-qPCR as described above. RT for precursor and mRNAs in MDA-MB-231 cells was done using the High Flex buffer in the miScript II RT kit.

Western blotting. Cells were grown in 6-well plates to ˜50% confluency in complete growth medium and then incubated with 500 nM of 2 or 5 for 48 h. Total protein was extracted using M-PER Mammalian Protein Extraction Reagent (Pierce Biotechnology) supplemented with 1× Protease Inhibitor cocktail (Roche). Extracted total protein was quantified using a Micro BCA Protein Assay Kit (Pierce Biotechnology). Approximately 10 μg of total protein was resolved using an 8% SDS-polyacrylamide gel and then transferred to a PVDF membrane for 80 min at 350 mA current (25 mM Tris, pH 8.5, 200 mM glycine and 20% (v/v) Methanol). The membrane was briefly washed with 1× Tris-buffered saline (TBS; 50 mM Tris-Cl, pH 7.5. 150 mM NaCl) and blocked with 5% milk in 1×TBST (1×TBS containing 0.05% Tween-20) for 1 h at room temperature. The membrane was then incubated with 1:1000 ZBTB4 primary antibody (Life Technologies) in 1×TBST containing 5% milk overnight at 4° C. The membrane was washed with 1× Tris Buffered a Saline with 0.1% Tween-20 (TBST: 20 mM Tris-Base pH 7.6; 150 mM NaCl, 0.1% (v/v) Tween-20) and incubated with 1:2000 antirabbit IgG horseradish-peroxidase (Cell Signaling) secondary antibody conjugate in 1×TBST for 1 h at room temperature. After washing with 1×TBST, protein expression was quantified using SuperSignal West Pico Chemiluminescent Substrate (Pierce Biotechnology) per the manufacturer's protocol and exposed to X-Ray film. The membrane was then stripped using 1× Stripping Buffer (200 mM glycine, 1% Tween-20, and 0.1% SDS, pH 2.2) followed by washing in 1×TBST. The membrane was blocked and probed for β-actin following the same procedure described above using 1:5000 3-actin primary antibody (Cell Signaling) in 1×TBST containing 5% milk overnight at 4° C. The membrane was washed with 1×TBST and incubated with 1:10,000 anti-rabbit IgG horseradish-peroxidase secondary antibody conjugate (Cell Signaling) in 1×TBST for 1 h at room temperature. ImageJ software from the National Institutes of Health was used to quantify band intensities.

Using a similar method as mentioned above, STK4 (MST-1) levels were investigated in DU145 cells. Approximately 10 μg of protein was resolved on a 12.5% Bis-Tris polyacrylamide gel pH 6.8 with a 4% Bis-Tris pH 6.8 stacking layer at 150 V in 1× Running Buffer (50 mM MOPS, 50 mM Tris, pH 7.7, 1 mM EDTA, and 1% (w/v) SDS). The proteins were transferred to a PVDF membrane using the wet transfer method at 350 mA for 1 h. Membranes were blocked with 1×TBST containing 5% milk and then probed with 1:400 of Rabbit anti-Human STK4 (Cell Signaling—D889Q) overnight in TBST with 5% Milk followed by washing and probing with 1:5000 anti-rabbit-HRP (Cell Signaling) for 2 h at room temp. Bands were visualized as mentioned earlier. After stripping, j-Actin was probed as described earlier, and imaged. PD-L1 was probed in a similar manner using 1:1000 Rabbit anti-Human PD-L1 (Cell Signaling-E1L3N®) and 1:5000 anti-rabbit HRP.

Caspase 3/7 Glo Assay. DU145 cells were seeded into 96-well black clear bottom plates (Corning—89091-014) at 50% confluency (≈20,000 cells/well) and allowed to adhere overnight. The cells were then treated with 2, 5, or 6 at 1, 10, 100, and 500 nM or LNAs targeting the cluster and a Scrambled LNA at 50 nM for 24 h. LNAs were obtained from Qiagen with the miRCURY Power LNA backbone and uptake tag, and were treated to the cells directly without transfection. Caspase 3/7 activity was measured by using the Caspase 3/7 glow reagent (Promega) according to the manufacturers protocol. Luminescence was measured on a Molecular Devices M5 plate reader with an integration time of 500 ms.

Invasion assay. A Boyden chamber assay was used to assess invasion of MDA-MB-231 cells. Transwell inserts were coated with 100 μL of 0.5 mg/mL Matrigel (Fisher Scientific: CB40234) diluted with serum free growth media at 37° C. for 30 min. MDA-MB-231 cells (5×10⁴) pre-treated with vehicle, LNA, Scramble 2 or 5 in serum free growth medium were seeded at the upper chamber with complete growth medium at the bottom. After incubating at 37° C. for 16 h, medium in the bottom wells and inserts was removed. The inserts and bottom wells were washed twice with PBS and excess liquid was removed with cotton swabs. To the bottom well was added 400 μL of 4% paraformaldehyde and incubated at room temperature for 20 min. The wells and inserts were washed twice with PBS and then stained for 20 min by adding 400 μL of 0.1% (w/v) crystal violet solution (dissolved in 4% aqueous MeOH). The wells and inserts were washed twice with water and twice with 1×PBS. After drying, the invaded cells were imaged using a Leica DMI3000 B upright fluorescent microscope and counted manually.

In vitro Bleomycin cleavage assay. The template used for pre-miR-17 (SEQ ID NO:1 TCAAAGTGCTTACAGTGCAGGTAGTGATATGTGCATCTACTGCAGTGAAGGCACTTG TAGC) was PCR-amplified in 1×PCR Buffer, 2 μM forward primer (SEQ ID NO:2 GGCCGGATCCTAATACGACTCACTATAGGTCAAAGTGCTTACAGTGCAGG), 2 μM reverse primer(SEQ ID NO:3 GCTACAAGTGCCTTCACTG), 4.25 mM MgCl₂, 330 μM dNTPs, and 2 μL of Taq DNA polymerase in a 50 μL reaction. Cycling conditions were 95° C. for 30 s, 55° C. for 30° C., and 72° C. for 60 s. Pre-miR-17 was folded in 5 mM NaH₂PO₄ at 60° C. for 5 min and then cooled down slowly to room temperature on the benchtop. Different concentrations (10, 20, 50, 100, 200, 500 or 1000 nM) of 5 were preincubated with Fe²⁺ and added to the folded RNA. After the first addition, a second and third aliquot of Fe²⁺ was added at 30 and 60 min of incubation respectively at 37° C. The mixture was then incubated at 37° C. for 24 h and the final cleavage products were separated on a 15% denaturing polyacrylamide gel and imaged using a Bio-Rad PMI phosphorimager.

In vitro Bleomycin cleavage of DNA plasmid. Compound 5, 6 or bleomycin A5 (0, 10, 100, 500 or 1000 nM) were pre-activated with 1 eq of (NH₄)₂Fe(SO₄)₂.6H₂O and then 500 ng of a plasmid was added to a final volume of 20 μL. Another equivalent of (NH₄)₂Fe(SO₄)₂.6H₂O was added after 30 min and 60 min respectively. The mixture was loaded on 1% agarose with 6× loading dye and stained with ethidium bromide. Bands were quantified using ImageJ image analysis software.

Overexpression of the miR-17-92a-1 cluster. DU145 or MDA-MB-231 cells were grown to 80% confluency in a 100 mm dish followed by transfection with 2000 ng of a pcDNA-miR-17-92a-1 or empty pcDNA vector as described previously.⁷ After transfection, cells were seeded into 6-well or 12-well plates and allowed to adhere for 12 h before being treated with 2 or 5 for 24 h for analysis of RNA expression. Total RNA was extracted and analyzed as described above.

Lentiviral transduction of MDA-MB-231 or DU145 cells with shRNAs. DU145 or MDA-MB-231 cells were transduced to express shRNAs targeting STK4 or ZBTB4 respectively. The lentiviral particles were generated by co-transfection of HEK 293T cells with (i) anti-STK4 (NM_020899.3—Genecopoeia) or anti-ZBTB4 (NM_006282.4—Genecopoeia); (ii) packaging plasmid (psPAX2-Addgene); and (iii) envelop plasmid (pmD2.G—Addgene) using Lipofectamine 3000 according to the manufacturers protocol in a ratio of (1.0:0.55:1.3 pmol). After removal of transfection media, media supernatants were harvested at 12, 24, and 48 h. Virus particles were concentrated using the Lenti-X Concentrator (Takara Biosciences) according to the manufacturers protocol. The viral pellet was resuspended in 1 mL of 1×DPBS and 300 μL was added to DU145 or MDA-MB-231 cells (˜50% confluency), which were allowed to grow for 48 h. Cells were split twice and then sorted using a BD-FACS Aria Fusion™ cell sorter to isolate mCherry positive cells. These cells were then grown for RT-qPCR, Western, and Caspase 3/7 analysis of shRNA expression's effect on compound efficacy and phenotype.

Chem-CLIP/Competitive-Chem-CLIP. DU145 cells were grown in 100 mm dishes to ˜80% confluency in complete growth medium. They were then treated with 3 or 4 for 6 h at 37° C. followed by washing once with 1×DPBS and then irradiated with 365 nm light for 10 min in ice fold DPBS. Cells were then scraped from the dish, pelleted, and the supernatants removed. Total RNA was extracted using the miRNeasy Mini kit (Qiagen) with DNase treatment according to the manufacturers protocol. To add a biotin handle onto RNA that has reacted with 3 or 4, 60 μg of total RNA was treated with 200 μL of Disulfide Azide Agarose beads (Click Chemistry Tools—1238-2) washed with 1×HEPES buffer (25 mM, pH 7) and 30 μL of (1:1:1) of 250 mM sodium ascorbate, 10 mM CuSO₄, 50 mM THPTA added in that order to a 500 μL final volume in 1×HEPES buffer. Tubes were incubated at 37° C. for 2 h followed by centrifugation. The beads were then washed six times with 1×Wash Buffer (10 mM Tris-HCl, pH 7.0, 4 M NaCl, 1 mM EDTA, and 0.2% (v/v) Tween-20) followed by two washes with nano pure water. Bound RNA was cleaved by treating the beads with 200 μL of 1:1 TCEP (200 mM) pre-reduced with K₂CO₃ (600 mM) for 30 min at 37° C. followed by quenching with 1 volume of iodoacetamide (400 mM) for 30 min at room temperature. The supernatants were removed, and the beads washed once with Nano pure water and combined with the supernatants, which were then concentrated by vacuum to 100 μL and the RNA cleaned up using RNA clean XP beads per the manufacturer's protocol. This RNA was then subjected to RT-qPCR analysis to measure enrichment of pri-miR-17-92 and pre-miR-17, which was calculated as the ratio of levels after pulldown to before pulldown described previously.⁸

Proteomics analysis of DU145 cells treated with 5. DU145 cells were grown in 100 mm dishes in growth medium and treated with 5 at 500 nM or vehicle (DMSO) for 24 h. After the treatment period, the cells were scraped from the dish and pelleted. The cells were re-suspended in 1×DPBS and pelleted; this step was repeated. The cells were lysed in 1×DPBS by sonication using Digital Sonifier SFX 150 (Branson). Protein concentration in lysates was measured using the Bradford assay (BioRad). An equal amount of protein from each sample (30 μg) was then denatured in 6 M urea in 50 mM NH₄HCO₃ (pH 8), reduced with 10 mM tris(2-carboxyethyl)phosphine hydrochloride (TCEP) for 30 min, and alkylated with 25 mM iodoacetamide for 30 min; the alkylation step was completed in the dark. Samples were diluted to 2 M urea with 50 mM NH₄HCO₃, and digested with trypsin (Thermo Scientific, 1.5 μL of 0.5 μg/μL) in the presence of 1 mM CaCl₂) for 12 h at 37° C. Samples were acidified with acetic acid to a final concentration of 5% (v/v), desalted over a self-packed C18 spin column, and dried using micro IR vacuum concentrator (CentriVap). Samples were analyzed by LC-MS/MS (see below), and the MS data were processed with MaxQuant (see below).

LC-MS/MS Analysis. Peptides were resuspended in water with 0.1% (v/v) formic acid (FA) and analyzed using EASY-nLC 1200 nano-UHPLC coupled to Q Exactive HF-X Quadrupole-Orbitrap mass spectrometer (Thermo Scientific). The chromatography column consisted of a 50 cm long, 75 μm i.d. microcapillary capped by a 5 μm tip and packed with ReproSil-Pur 120 C18-AQ 2.4 μm beads (Dr. Maisch GmbH). LC solvents were 0.1% FA in H₂O (Buffer A) and 0.1% FA in 90% MeCN:10% H₂O (Buffer B). Peptides were eluted into the mass spectrometer at a flow rate of 300 nL/min over a 240 min linear gradient (5-35% Buffer B) at 65° C. Data were acquired in data-dependent mode (top-20, NCE 28, R=7500) after full MS scan (R=60000, m/z 400-1300). Dynamic exclusion was set to 10 s, peptide match set to prefer, and isotope exclusion was enabled.

MaxQuant Analysis. The mass spectrometer data were analyzed with MaxQuant⁹ (V1.6.1.0) and searched against the human proteome (Uniprot) and a common list of contaminants (included in MaxQuant). The first peptide search tolerance was set at 20 ppm; 10 ppm was used for the main peptide search, and fragment mass tolerance was set to 0.02 Da. The false discovery rate for peptides, proteins, and sites identification was set to 1%. The minimum peptide length was set to six amino acids, and peptide re-quantification, label-free quantification (MaxLFQ), and “match between runs” were enabled. The minimal number of peptides per protein was set to 2. Methionine oxidation was searched as a variable modification, and carbamidomethylation of cysteines was searched as a fixed modification.

PD-L1 Overexpression Analysis. DU145 cells were seeded into 60 mm dishes and grown to a ˜70% confluency. Then, they were transfected with 200, 1000, 2000, or 4000 ng of the pGIPZ-PD-L1-EGFP plasmid to overexpress PD-L1 for 24 h. Total RNA was extracted to assess the change in mRNA levels required to alter surface PD-L1 expression. Cell surface expression was measured by scraping transfected cells from the 60 mm dish and then washing them once with 1×DPBS. They were then resuspended in Buffer 1 (1×DPBS containing 5% (v/v) FBS and 1% (w/v) NaN₃) Next, 1 volume of 1×DPBS with 5% BSA was added, and the cells were incubated for 15 min followed by addition of anti-PD-L1-Alexa 647 conjugate (Cell Signaling-417265; final dilution of 1:50). The cells were incubated at room temperature in the dark with the antibody for 1.5 h followed by three washes with 1×DPBS before resuspension in Buffer 1 for Fluorescence Assisted Cell Sorting (FACS) analysis. Cells were analyzed on a BD-FACS LSRII using standard laser parameters for Alexa-647 expression. FACS data and plots were analyzed on FlowJo 6, and the mean at maximum intensity was used for plotting the data.

Cellular uptake analysis. DU145 and MDA-MB-231 cells were seeded into a 96-well white clear bottom plate at 10,000 cells/well and allowed to adhere overnight. Once adhered, the cells were grown to ˜50% confluency and then treated with 2, 5, or 7 at 5 μM for 24 h while also leaving untreated wells for generation of a standard curve. This concentration was chosen to allow for adequate signal above noise. After 24 h, cells were lysed in 100 μL of RNA lysis buffer (Zymo Research) for 5 min. Compound 2, 5, or 7 were spiked into untreated samples at 100, 10, 1, 0.1, and 0.01 nM to create a standard curve of compound fluorescence. Using a Biotek FLX-800 fluorescence plate reader (excitation: 360/340; emission 460/440; sensitivity=90) the fluorescence of 2, 5, and 7 was measured. Concentrations were determined by extrapolating from the standard curves mentioned above.

Cellular localization of 2, 5, and 7 in DU145 and MDA-MB-231 cells. DU145 and MDA-MB-231 cells were seeded into a poly-D-lysine coated glass bottom 35 mm dishes (MatTek). Cells were then treated with 2, 5, or 7 (5 μM) for 24 h. After incubation, cells were washed with PBS twice and the nucleus stained with Syto 82 for 20 min in 1× indicator free RPMI 1640 (Gibco). Images were taken on an Olympus FluoView 1000 confocal microscope at 100× magnification in 1× indicator free RPMI 1640 and images were overlayed in the Olympus FluoView software to determine co-localization of compounds with cellular compartments. Brightness and Contrast were adjusted to settings of 84 and −49, respectively, in Adobe Photoshop for all images.

Absolute quantification of pri-, pre-, and mature miRNAs. Transcripts of pre-miR-17, pre-miR-18a, and the corresponding 5p mature sequences were transcribed in vitro and purified as described above. Precursor miRNAs (1×10¹⁴ copies) were reverse transcribed using QScript RT (Quanta bio) in a total volume of 40 μL. Mature miRNAs (1×10¹⁴ copies) were reverse transcribed using the miScript II RT Kit (Qiagen) in a total volume of 40 μL reaction. Serial dilutions of the RT reactions (1:10) were used to create a standard curve of copy number versus Cₜ which was used to calculate copy numbers of each transcript in DU-145 and MDA-MB-231 cells.

### Synthetic Methods and Characterization

### Abbreviations

### DCM: Dichloromethane

### DIC: N,N′-Diisopropylcarbodiimide

DIEA: Diisopropyl ethyl amine

### DMF: Dimethylformamide

DMSO: Dimethyl sulfoxide
EDTA: Ethylenediaminetetraacetic acid
HATU: Hexafluorophosphate azabenzotriazole tetremethyl uronium
HOAt: 1-hydroxy-7-azabenzotriazole

### HPLC: High-performance Liquid Chromatography

### MeOH: Methanol

### TFA: Trifluoro Acetic Acid

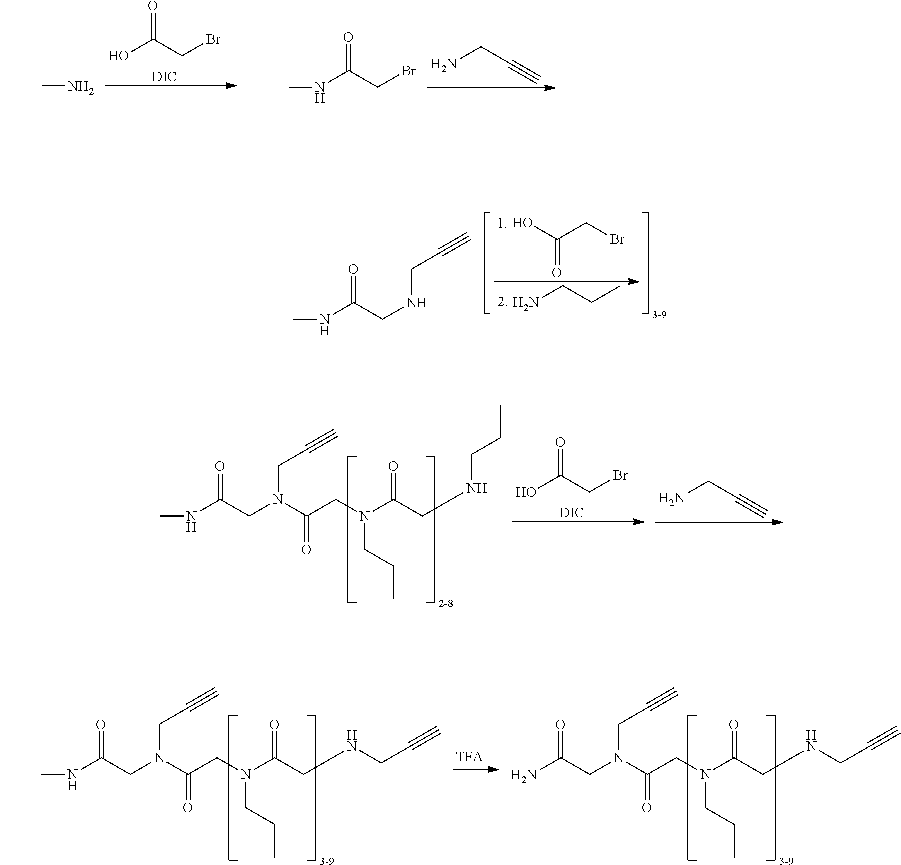

General Protocol for Peptoid Synthesis: Peptoids were synthesized via standard resin-supported oligomerization protocol. Rink resin (555 mg, 0.6 mmol) was activated with 20% piperidine in DMF for 30 min. After that, solvent was removed and washed with DMF and DCM for 3 times respectively.

Coupling Step: To the resin was added 3 mL of 1 M bromoacetic acid in DCM (3 mmol, 5 eq) and DIC (3.0 mmol, 519 μL). The resin was shaken at room temperature for 2 h. Then the solvent was removed, and the resin was washed with DMF for three times.

Displacement step: To the resin was added 5 mL DMF and propargylamine. The resin was shaken at room temperature for 2 h. Then the solvent was removed, and the resin was washed with DMF for three times.

Peptoid Chain Extension: a) To the resin was added 5 mL DMF, bromoacetic acid and DIC. The resin was shaken at room temperature for 2 h. Then the solvent was removed, and the resin was washed with DMF for three times. b) To the resin was added 5 mL DMF and propyl amine. The resin was shaken at room temperature for 2 h. Then the solvent was removed, and the resin was washed with DMF for three times. Steps a) and b) were repeated for another 2-9 times.

Cleavage of the peptoid: The resin was treated with 30% TFA in DCM and shaken at room temperature for 30 min. The solution was collected and concentrated in vacuo. The residue was purified by HPLC.

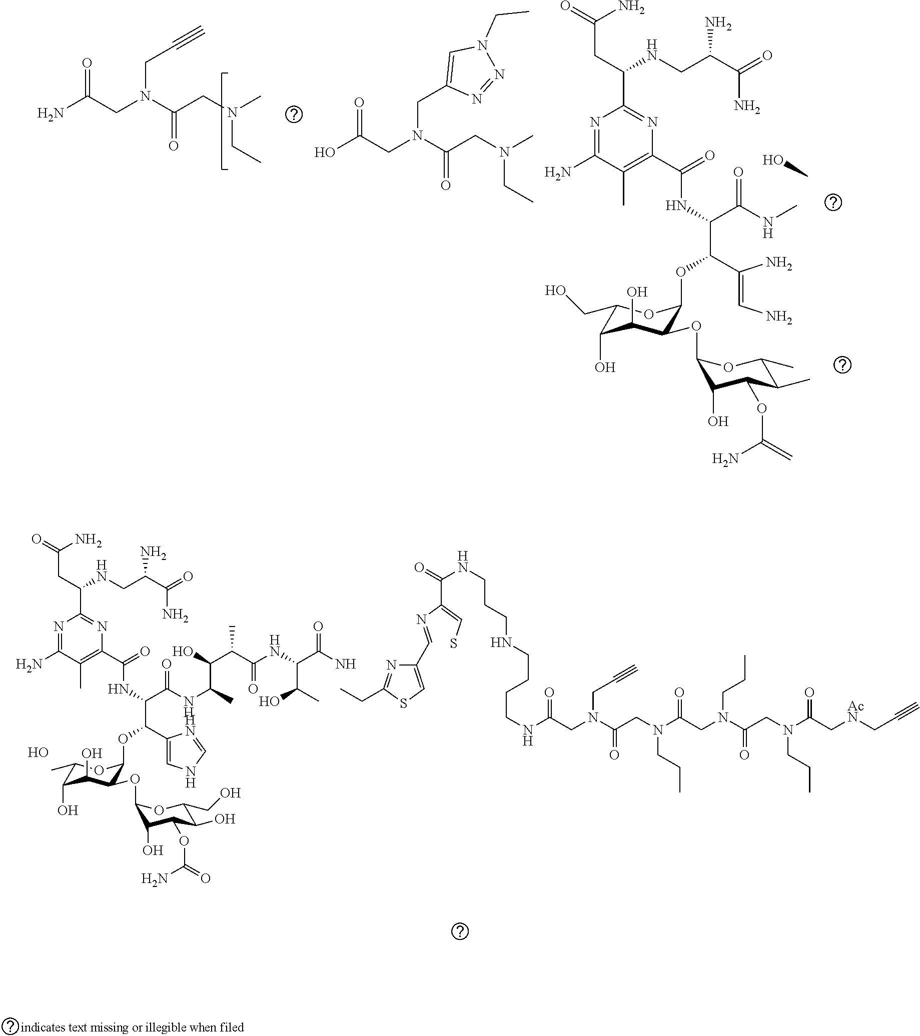

General procedure for the click chemistry: A solution of the peptoid (1 eq), Monomer (2 eq), CuSO₄.5H₂O (2 eq) and ascorbic acid (2 eq) in DMF was stirred at room temperature overnight. The resulting mixture was purified by HPLC to afford the corresponding dimer.

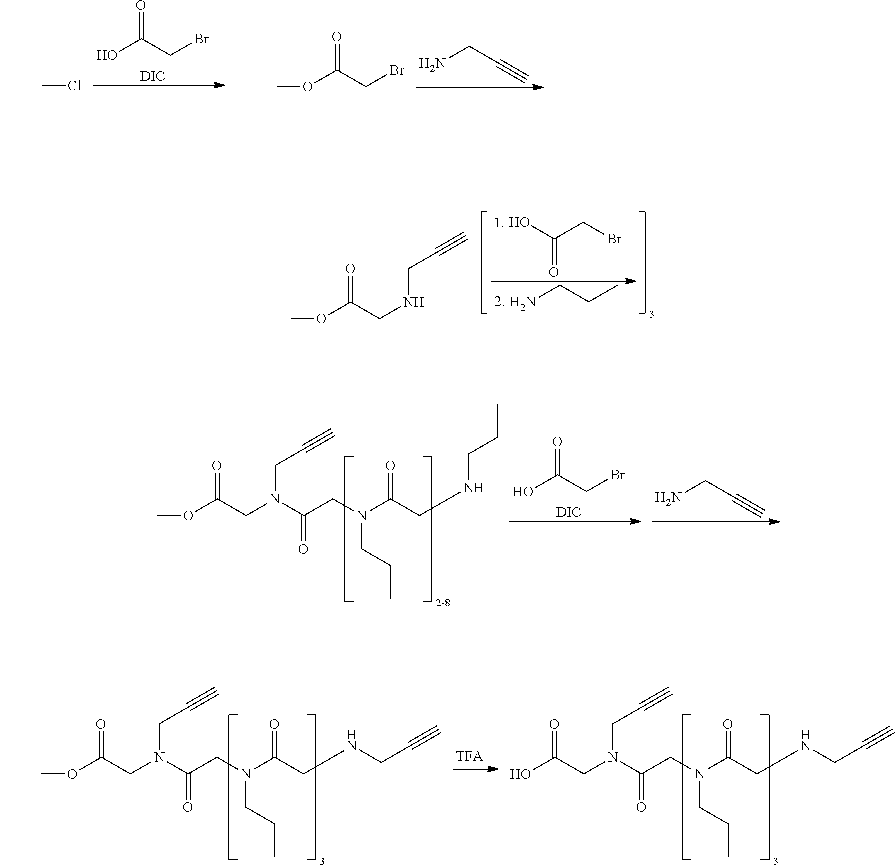

General Protocol for Peptoid Synthesis: Peptoids were synthesized via standard resin-supported oligomerization protocol. Chloro trityl resin (555 mg, 0.6 mmol) was activated with 1 M HCl/dioxane in DCM (4 M HCl dioxane was diluted with DCM) for 30 min. After that, solvent was removed and washed with DMF and DCM for 3 times respectively.

Coupling Step: To the resin was added 3 mL of 1 M bromoacetic acid in DCM (3 mmol, 5 eq) and DIC (3.0 mmol, 519 μL). The resin was shaken at room temperature for 2 h. The solvent was removed, and the resin was washed with DMF for three times.

Displacement step: To the resin was added 5 mL DMF and propargylamine. The resin was shaken at room temperature for 2 h. Then the solvent was removed, and the resin was washed with DMF for three times.

Peptoid Chain Extension: a) To the resin was added 5 mL DMF, bromoacetic acid and DIC. The resin was shaken at room temperature for 2 h. Then the solvent was removed, and the resin was washed with DMF for three times. b) To the resin was added 5 mL DMF and propylamine. The resin was shaken at room temperature for 2 h. Then the solvent was removed, and the resin was washed with DMF for three times. Steps a) and b) were repeated twice.

Cleavage of the peptoid: The resin was treated with 30% TFA in DCM and shaken at room temperature for 30 min. The solution was collected and concentrated in vacuo. The residue was purified by HPLC.

### Synthesis of 5 and 6

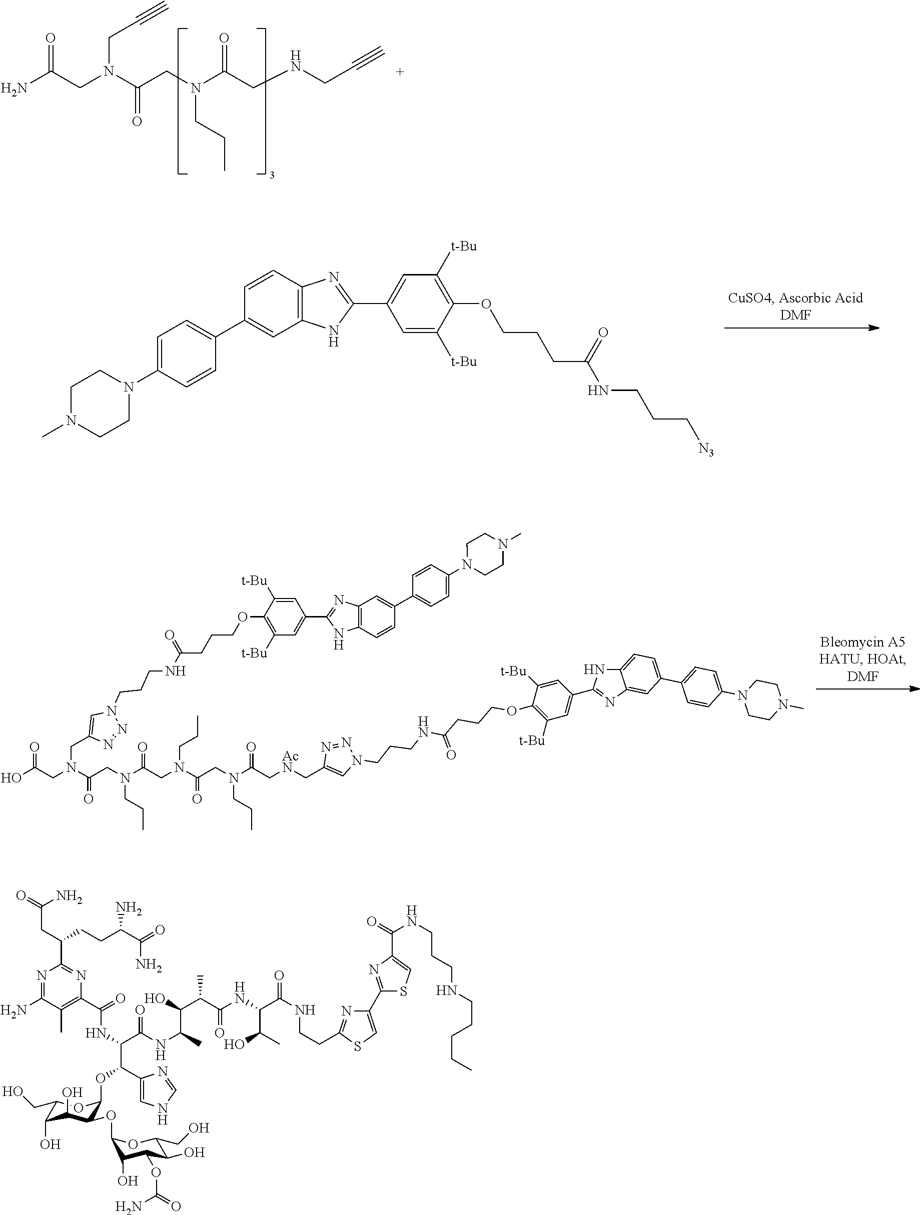

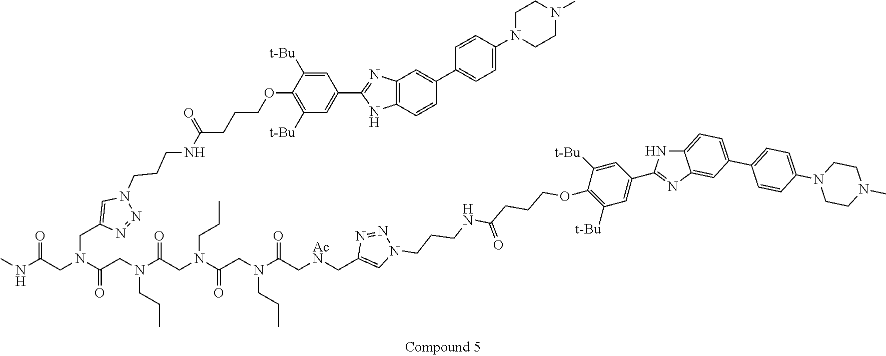

The Dimer acid was obtained by the general click reaction as described above. The acid was preincubated with HATU (1.5 equiv.), HOAt (1.5 equiv.) and DIEA (1.5 equiv.) in DMF for 10 min. Then a solution of Bleomycin A5 (3 equiv.) in DMSO was added. The mixture was stirred at room temperature for 2 h and then the mixture was subjected to HPLC purification. After injection of the sample, the column was washed with 50 mM EDTA (pH 6.7) for 15 min to remove copper ion and then water for another 15 min to remove EDTA. 5 was purified with a linear gradient from 0 to 100% B (MeOH+0.1% TFA) in A (water+0.1% TFA) over 60 min at a flow rate of 5 mL/min. MALDI: [M+H]+ calculated: 3297.7249, [M+H]+ observed: 3298.9922. Synthesis of 2-FAM

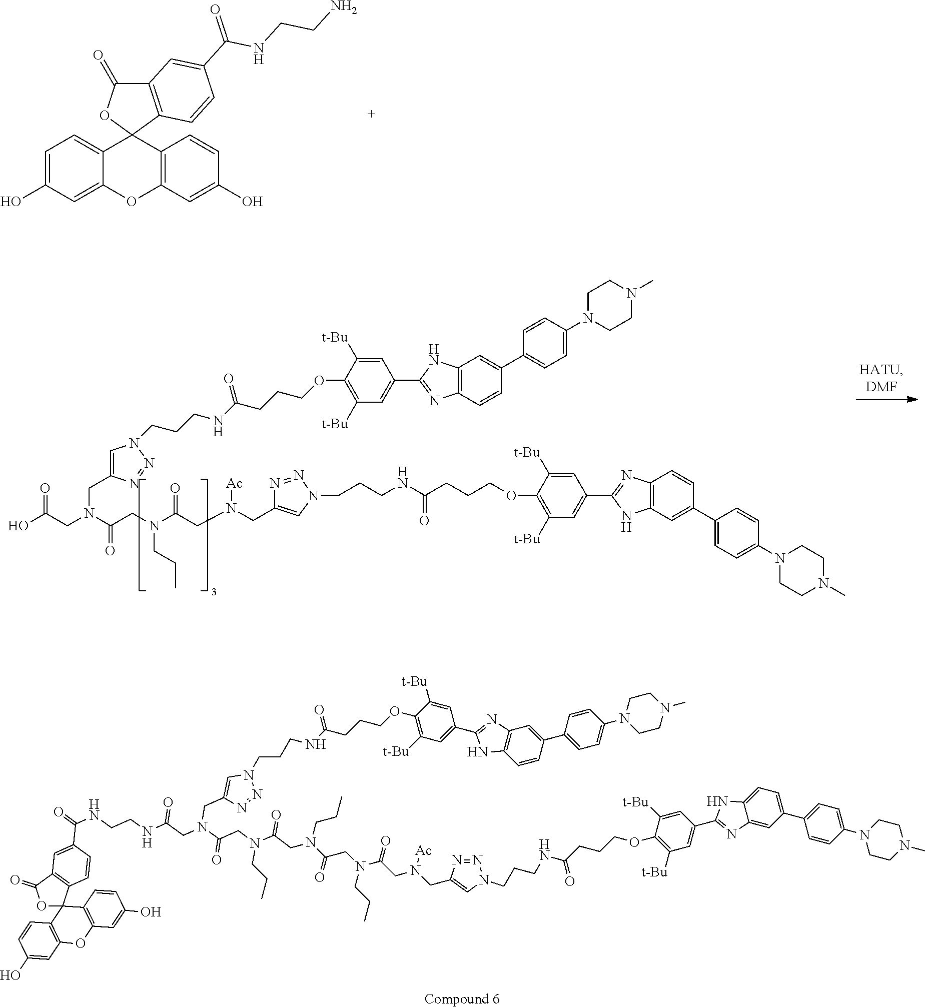

A solution of the Dimer acid was incubated with HATU (1.5 equiv.) at room temperature for 10 min and then the FAM amine was added followed by the addition of 5 equiv. of DIEA and the mixture was stirred at room temperature for another 2 h. 2-FAM was purified by HPLC. MALDI: [M+K]+ calculated: 2316.9388, [M+K]⁺ observed: 2317.3967.

### Synthesis of 7

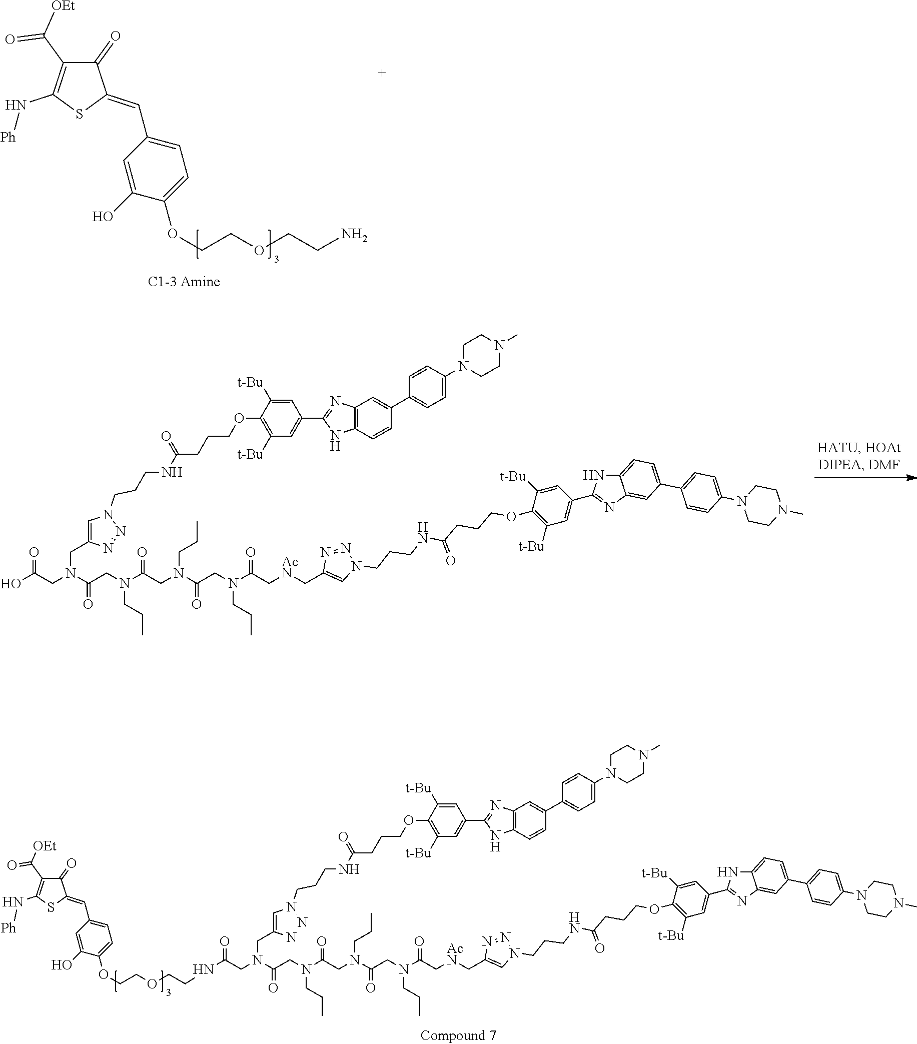

To a solution of the Dimer acid in DMSO (12 mM, 90 μL, 1.08 μmol) was added a mixture of HATU (0.62 mg, 1.5 μmol) and HOAt (0.22 mg, 1.5 μmol) in DMF (5 μL), and the solution was stirred for 10 min at room temperature. A mixture of C1-3 amine (1.2 mg, 2.02 μmol), synthesized as previously described,¹⁰ and DIPEA (0.94 μL, 5.4 μmol) in DMF (12 μL) was added to the solution and stirred overnight. After dilution with 30% MeOH/H₂O (0.1% TFA), the product was purified by HPLC (70-90% MeOH/H₂O in 30 min, 0.1% TFA) to give 7 (0.3 mg, 0.12 μmol, 11%). HR-MS (ESI) calculated. for C₁₃₃H₁₇₆N₂₃O₁₈S⁻ [M−H]⁻: 2415.3290; observed: 2415.3236.

### HR MS and HPLC Characterizing Data For Compounds 1D, 2, 4FL, 5 and 7

Compound 2 (also Compound 1D, n=1) HR MS=1834.0452; HPLC=42 min. (minutes, HPLC conditions described above)
Compound 1D, n=2; HR MS=1933.1505 mw; HPLC=43.4 min.
Compound 1D, n=3; HR MS=2032.2262 mw; HPLC=42.4 min.
Compound 1D, n=4; HR MS=2131.3394 mw; HPLC=42.2 min.
Compound 1D, n=5; HR MS=2230.2078 mw; HPLC=43.6 min.
Compound 1D, n=6; HR MS=2329.3686 mw; HPLC=44 min.
Compound 1D, n=7; HR MS=2428.3708 mw; HPLC=44 min.

### Compound 5; HR MS=3298.9922; HPLC=41.2 min.

### Compound 4FL; HR MS=2317.3967 mw; HPLC=44.2 min.

### Compound 6; HR MS=1970.0596; HPLC=28.25 min.

Compound 7; FBF (Counts v. mass to charge) 2416.32755 mw; HPLC=46.0

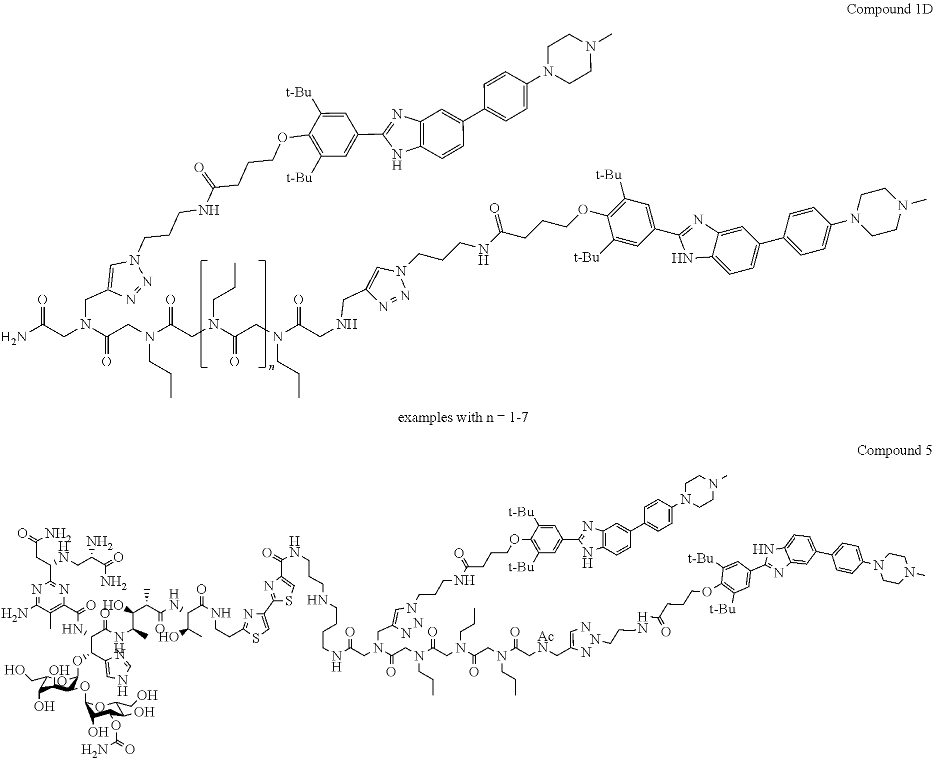

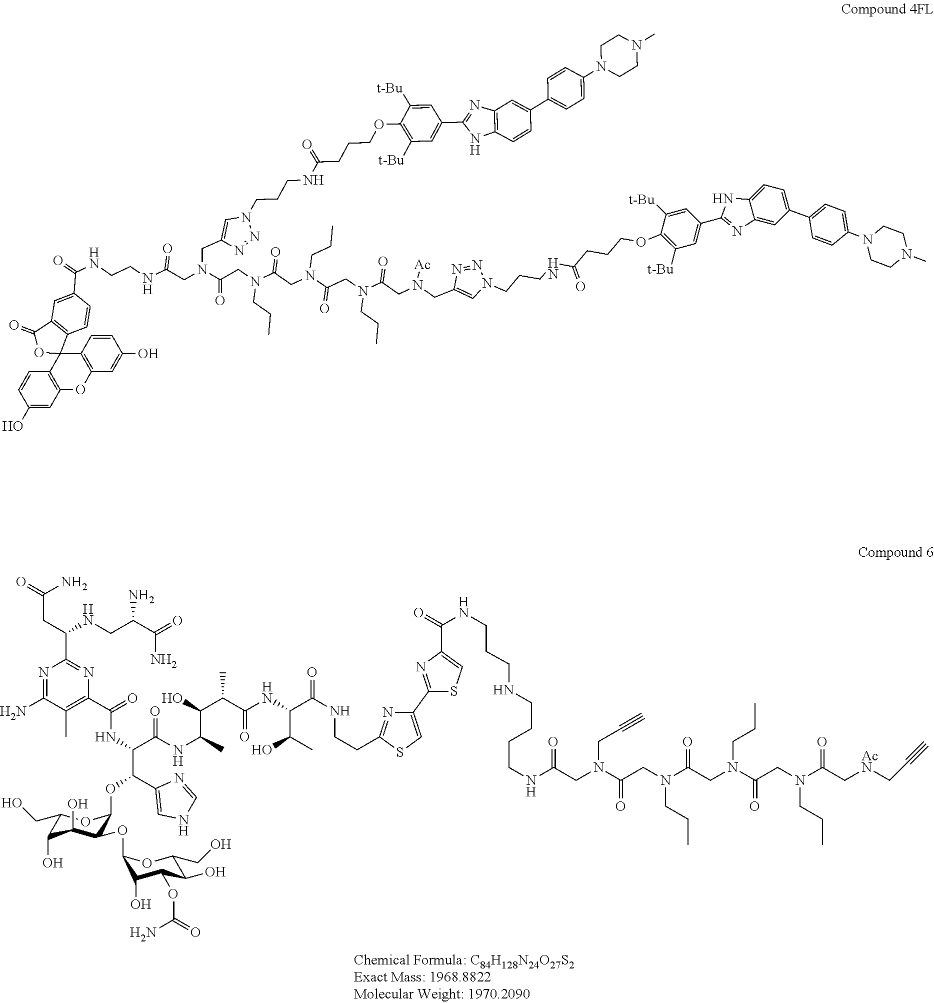

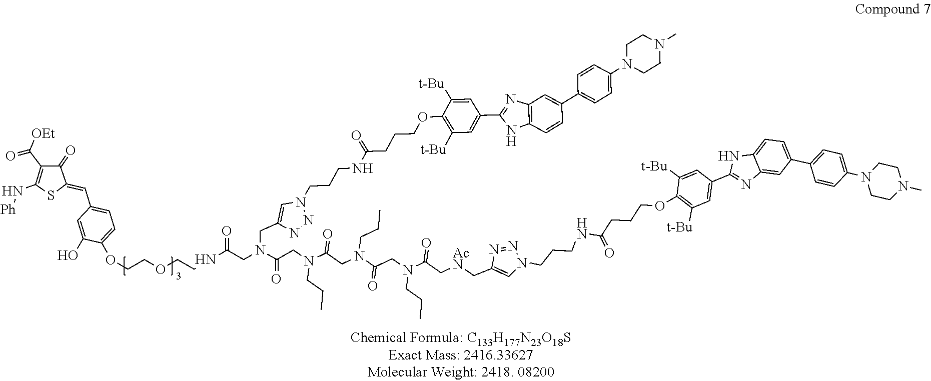

### REFERENCES

1. Agrawal, S.; Goodchild, J.; Civeira, M. P.; Thornton, A. H.; Sarin, P. S.; Zamecnik, P. C., Oligodeoxynucleoside phosphoramidates and phosphorothioates as inhibitors of human immunodeficiency virus. Proc. Natl. Acad. Sci. U.S.A. 1988, 85 (19), 7079-83.
2. Zamecnik, P. C.; Stephenson, M. L., Inhibition of Rous sarcoma virus replication and cell transformation by a specific oligodeoxynucleotide. Proc. Natl. Acad. Sci. U.S.A. 1978, 75 (1), 280-4.
3. Costales, M. G.; Matsumoto, Y.; Velagapudi, S. P.; Disney, M. D., Small molecule targeted recruitment of a nuclease to RNA. J. Am. Chem. Soc. 2018, 140 (22), 6741-6744.
4. Davis, M. E.; Zuckerman, J. E.; Choi, C. H.; Seligson, D.; Tolcher, A.; Alabi, C. A.; Yen, Y.; Heidel, J. D.; Ribas, A., Evidence of RNAi in humans from systemically administered siRNA via targeted nanoparticles. Nature 2010, 464 (7291), 1067-70.
5. Stein, C. A.; Castanotto, D., FDA-approved oligonucleotide therapies in 2017. Mol. Ther. 2017, 25 (5), 1069-1075.
6. Abudayyeh, O. O.; Gootenberg, J. S.; Essletzbichler, P.; Han, S.; Joung, J.; Belanto, J. J.; Verdine, V.; Cox, D. B. T.; Kellner, M. J.; Regev, A.; Lander, E. S.; Voytas, D. F.; Ting, A. Y.; Zhang, F., RNA targeting with CRISPR-Cas13. Nature 2017, 550 (7675), 280-284.
7. Lima, W. F.; Vickers, T. A.; Nichols, J.; Li, C.; Crooke, S. T., Defining the factors that contribute to on-target specificity of antisense oligonucleotides. PLoS One 2014, 9 (7), e101752.
8. Stombaugh, J.; Zirbel, C. L.; Westhof, E.; Leontis, N. B., Frequency and isostericity of RNA base pairs. Nucleic Acids Res. 2009, 37 (7), 2294-312.
9. Crews, L. A.; Balaian, L.; Delos Santos, N. P.; Leu, H. S.; Court, A. C.; Lazzari, E.; Sadarangani, A.; Zipeto, M. A.; La Clair, J. J.; Villa, R.; Kulidjian, A.; Storb, R.; Morris, S. R.; Ball, E. D.; Burkart, M. D.; Jamieson, C. H. M., RNA splicing modulation selectively impairs leukemia stem cell maintenance in secondary human AML. Cell Stem Cell 2016, 19 (5), 599-612.
10. Tor, Y., Targeting RNA with small molecules. Chembiochem 2003, 4 (10), 998-1007.
11. Disney, M. D., Targeting RNA with small molecules to capture opportunities at the intersection of chemistry, biology, and medicine. J. Am. Chem. Soc. 2019, 141 (17), 6776-6790.
12. Connelly, C. M.; Moon, M. H.; Schneekloth, J. S., Jr., The emerging role of RNA as a therapeutic target for small molecules. Cell Chem Biol 2016, 23 (9), 1077-1090.
13. Leon, B.; Kashyap, M. K.; Chan, W. C.; Krug, K. A.; Castro, J. E.; La Clair, J. J.; Burkart, M. D., A challenging pie to splice: drugging the spliceosome. Angew. Chem. Int. Ed. Engl. 2017, 56 (40), 12052-12063.
14. Disney, M. D.; Winkelsas, A. M.; Velagapudi, S. P.; Southern, M.; Fallahi, M.; Childs-Disney, J. L., Inforna 2.0: A Ppatform for the sequence-based design of small molecules targeting structured RNAs. ACS Chem. Biol. 2016, 11 (6), 1720-8.
15. Velagapudi, S. P.; Gallo, S. M.; Disney, M. D., Sequence-based design of bioactive small molecules that target precursor microRNAs. Nat. Chem. Biol. 2014, 10 (4), 291-+.
16. Wang, Z. F.; Ursu, A.; Childs-Disney, J. L.; Guertler, R.; Yang, W. Y.; Bernat, V.; Rzuczek, S. G.; Fuerst, R.; Zhang, Y. J.; Gendron, T. F.; Yildirim, I.; Dwyer, B. G.; Rice, J. E.; Petrucelli, L.; Disney, M. D., The Hairpin Form of r(G4C2)(exp) in c9ALS/FTD Is Repeat-Associated Non-ATG Translated and a Target for Bioactive Small Molecules. Cell Chem Biol. 2019, 26 (2), 179-190 e12.
17. Costales, M. G.; Haga, C. L.; Velagapudi, S. P.; Childs-Disney, J. L.; Phinney, D. G.; Disney, M. D., Small molecule inhibition of microRNA-210 reprograms an oncogenic hypoxic circuit. J. Am. Chem. Soc. 2017, 139 (9), 3446-3455.
18. Velagapudi, S. P.; Costales, M. G.; Vummidi, B. R.; Nakai, Y.; Angelbello, A. J.; Tran, T.; Haniff, H. S.; Matsumoto, Y.; Wang, Z. F.; Chatterjee, A. K.; Childs-Disney, J. L.; Disney, M. D., Approved anti-cancer drugs target oncogenic non-coding RNAs. Cell Chem Biol. 2018, 25 (9), 1086-1094 e7.
19. Angelbello, A. J.; Rzuczek, S. G.; McKee, K. K.; Chen, J. L.; Olafson, H.; Cameron, M. D.; Moss, W. N.; Wang, E. T.; Disney, M. D., Precise small-molecule cleavage of an r(CUG) repeat expansion in a myotonic dystrophy mouse model. Proc. Natl. Acad. Sci. U.S.A. 2019, 116 (16), 7799-7804.
20. Velagapudi, S. P.; Cameron, M. D.; Haga, C. L.; Rosenberg, L. H.; Lafitte, M.; Duckett, D. R.; Phinney, D. G.; Disney, M. D., Design of a small molecule against an oncogenic noncoding RNA. Proc. Natl. Acad. Sci. U.S.A. 2016, 113 (21), 5898-903.
21. Mu, P.; Han, Y. C.; Betel, D.; Yao, E.; Squatrito, M.; Ogrodowski, P.; de Stanchina, E.; D'Andrea, A.; Sander, C.; Ventura, A., Genetic dissection of the miR-17-92 cluster of microRNAs in Myc-induced B-cell lymphomas. Genes Dev. 2009, 23 (24), 2806-11.
22. Conkrite, K.; Sundby, M.; Mukai, S.; Thomson, J. M.; Mu, D.; Hammond, S. M.; MacPherson, D., miR-17-92 cooperates with RB pathway mutations to promote retinoblastoma.

Genes. Dev. 2011, 25 (16), 1734-45.
23. Huang, G.; Nishimoto, K.; Zhou, Z.; Hughes, D.; Kleinerman, E. S., miR-20a encoded by the miR-17-92 cluster increases the metastatic potential of osteosarcoma cells by regulating Fas expression. Cancer Res. 2012, 72 (4), 908-16.
24. Kim, K.; Chadalapaka, G.; Lee, S. O.; Yamada, D.; Sastre-Garau, X.; Defossez, P. A.; Park, Y. Y.; Lee, J. S.; Safe, S., Identification of oncogenic microRNA-17-92/ZBTB4/specificity protein axis in breast cancer. Oncogene 2012, 31 (8), 1034-44.
25. Li, Y.; Chen, M.; Liu, J.; Li, L.; Yang, X.; Zhao, J.; Wu, M.; Ye, M., Upregulation of microRNA 18b contributes to the development of colorectal cancer by inhibiting CDKN2B. Mol. Cell Biol. 2017, 37 (22), e00391-17.
26. Topkara, V. K.; Mann, D. L., Role of microRNAs in cardiac remodeling and heart failure. Cardiovasc. Drugs Ther. 2011, 25 (2), 171-82.
27. Mogilyansky, E.; Rigoutsos, I., The miR-17/92 cluster: a comprehensive update on its genomics, genetics, functions and increasingly important and numerous roles in health and disease. Cell Death Differ. 2013, 20 (12), 1603-14.
28. Kabekkodu, S. P.; Shukla, V.; Varghese, V. K.; J, D. S.; Chakrabarty, S.; Satyamoorthy, K., Clustered miRNAs and their role in biological functions and diseases. Biol. Rev. Camb. Philos. Soc. 2018, 93 (4), 1955-1986.
29. He, L.; Thomson, J. M.; Hemann, M. T.; Hernando-Monge, E.; Mu, D.; Goodson, S.; Powers, S.; Cordon-Cardo, C.; Lowe, S. W.; Hannon, G. J.; Hammond, S. M., A microRNA polycistron as a potential human oncogene. Nature 2005, 435 (7043), 828-33.
30. Thayanithy, V.; Sarver, A. L.; Kartha, R. V.; Li, L.; Angstadt, A. Y.; Breen, M.; Steer, C. J.; Modiano, J. F.; Subramanian, S., Perturbation of 14q32 miRNAs-cMYC gene network in osteosarcoma. Bone 2012, 50 (1), 171-81.
31. Sage, J.; Ventura, A., miR than meets the eye. Genes Dev. 2011, 25 (16), 1663-1667.
32. Bonauer, A.; Carmona, G.; Iwasaki, M.; Mione, M.; Koyanagi, M.; Fischer, A.; Burchfield, J.; Fox, H.; Doebele, C.; Ohtani, K.; Chavakis, E.; Potente, M.; Tjwa, M.; Urbich, C.; Zeiher, A. M.; Dimmeler, S., MicroRNA-92a controls angiogenesis and functional recovery of ischemic tissues in mice. Science 2009, 324 (5935), 1710-3.
33. Tsitsiou, E.; Lindsay, M. A., microRNAs and the immune response. Curr. Opin. Pharmacol. 2009, 9 (4), 514-20.
34. Velagapudi, S. P.; Disney, M. D., Two-dimensional combinatorial screening enables the bottom-up design of a microRNA-10b inhibitor. Chem Commun. 2014, 50 (23), 3027-9.
35. Velagapudi, S. P.; Luo, Y.; Tran, T.; Haniff, H. S.; Nakai, Y.; Fallahi, M.; Martinez, G. J.; Childs-Disney, J. L.; Disney, M. D., Defining RNA-small molecule affinity landscapes enables design of a small molecule inhibitor of an oncogenic noncoding RNA. ACS Cent. Sci. 2017, 3 (3), 205-216.
36. Chan, T. R.; Hilgraf, R.; Sharpless, K. B.; Fokin, V. V., Polytriazoles as copper(I)-stabilizing ligands in catalysis. Org. Lett. 2004, 6 (17), 2853-5.
37. Kolb, H. C.; Finn, M. G.; Sharpless, K. B., Click chemistry: diverse chemical function from a few good reactions. Angew. Chem. Int. Ed. Engl. 2001, 40 (11), 2004-2021.
38. Childs-Disney, J. L.; Tsitovich, P. B.; Disney, M. D., Using modularly assembled ligands to bind RNA internal loops separated by different distances. Chembiochem 2011, 12 (14), 2143-6.
39. Hajarnis, S.; Lakhia, R.; Yheskel, M.; Williams, D.; Sorourian, M.; Liu, X. Q.; Aboudehen, K.; Zhang, S. R.; Kersjes, K.; Galasso, R.; Li, J.; Kaimal, V.; Lockton, S.; Davis, S.; Flaten, A.; Johnson, J. A.; Holland, W. L.; Kusminski, C. M.; Scherer, P. E.; Harris, P. C.; Trudel, M.; Wallace, D. P.; Igarashi, P.; Lee, E. C.; Androsavich, J. R.; Patel, V., MicroRNA-17 family promotes polycystic kidney disease progression through modulation of mitochondrial metabolism. Nat. Commun. 2017, 8, 14395.
40. McGhee, J. D.; von Hippel, P. H., Theoretical aspects of DNA-protein interactions: co-operative and non-co-operative binding of large ligands to a one-dimensional homogeneous lattice. J. Mol. Biol. 1974, 86 (2), 469-89.
41. Chakraborty, S.; Mehtab, S.; Patwardhan, A.; Krishnan, Y., Pri-miR-17-92a transcript folds into a tertiary structure and autoregulates its processing. RNA (New York, N.Y.) 2012, 18 (5), 1014-28.
42. Chaulk, S. G.; Thede, G. L.; Kent, O. A.; Xu, Z.; Gesner, E. M.; Veldhoen, R. A.; Khanna, S. K.; Goping, I. S.; MacMillan, A. M.; Mendell, J. T.; Young, H. S.; Fahlman, R. P.; Glover, J. N., Role of pri-miRNA tertiary structure in miR-17-92 miRNA biogenesis. RNA Biol. 2011, 8 (6), 1105-14.
43. Lewis, B. P.; Burge, C. B.; Bartel, D. P., Conserved seed pairing, often flanked by adenosines, indicates that thousands of human genes are microRNA targets. Cell 2005, 120 (1), 15-20.
44. Loghman-Adham, M.; Nauli, S. M.; Soto, C. E.; Kariuki, B.; Zhou, J., Immortalized epithelial cells from human autosomal dominant polycystic kidney cysts. Am. J. Physiol. Renal. Physiol. 2003, 285 (3), F397-412.
45. Hsu, T. I.; Hsu, C. H.; Lee, K. H.; Lin, J. T.; Chen, C. S.; Chang, K. C.; Su, C. Y.; Hsiao, M.; Lu, P. J., MicroRNA-18a is elevated in prostate cancer and promotes tumorigenesis through suppressing STK4 in vitro and in vivo. Oncogenesis 2014, 3, e99.
46. Guan, L.; Disney, M. D., Covalent small-molecule-RNA complex formation enables cellular profiling of small-molecule-RNA interactions. Angew. Chem. Int. Ed. Engl. 2013, 52 (38), 10010-3.
47. Su, Z.; Zhang, Y.; Gendron, T. F.; Bauer, P. O.; Chew, J.; Yang, W. Y.; Fostvedt, E.; Jansen-West, K.; Belzil, V. V.; Desaro, P.; Johnston, A.; Overstreet, K.; Oh, S. Y.; Todd, P. K.; Berry, J. D.; Cudkowicz, M. E.; Boeve, B. F.; Dickson, D.; Floeter, M. K.; Traynor, B. J.; Morelli, C.; Ratti, A.; Silani, V.; Rademakers, R.; Brown, R. H.; Rothstein, J. D.; Boylan, K. B.; Petrucelli, L.; Disney, M. D., Discovery of a biomarker and lead small molecules to target r(GGGGCC)-associated defects in c9FTD/ALS. Neuron 2014, 83 (5), 1043-50.
48. Rzuczek, S. G.; Colgan, L. A.; Nakai, Y.; Cameron, M. D.; Furling, D.; Yasuda, R.; Disney, M. D., Precise small-molecule recognition of a toxic CUG RNA repeat expansion. Nat. Chem. Biol. 2017, 13 (2), 188-193.
49. Li, Y.; Disney, M. D., Precise small molecule degradation of a noncoding RNA identifies cellular binding sites and modulates an oncogenic phenotype. ACS Chem. Biol. 2018, 13 (11), 3065-3071.
50. Angelbello, A. J.; Disney, M. D., Bleomycin Can Cleave an Oncogenic Noncoding RNA. 2018, 19 (1), 43-47.
51. Krichevsky, A. M.; Gabriely, G., miR-21: a small multi-faceted RNA. J. Cell. Mol. Med. 2009, 13 (1), 39-53.
52. Zhou, B.; Irwanto, A.; Guo, Y. M.; Bei, J. X.; Wu, Q.; Chen, G.; Zhang, T. P.; Lei, J. J.; Feng, Q. S.; Chen, L. Z.; Liu, J.; Zhao, Y. P., Exome sequencing and digital PCR analyses reveal novel mutated genes related to the metastasis of pancreatic ductal adenocarcinoma. Cancer Biol. Ther. 2012, 13 (10), 871-9.
53. Casey, S. C.; Tong, L.; Li, Y.; Do, R.; Walz, S.; Fitzgerald, K. N.; Gouw, A. M.; Baylot, V.; Gutgemann, I.; Eilers, M.; Felsher, D. W., MYC regulates the antitumor immune response through CD47 and PD-L1. Science 2016, 352 (6282), 227-231.
54. Audrito, V.; Serra, S.; Stingi, A.; Orso, F.; Gaudino, F.; Bologna, C.; Neri, F.; Garaffo, G.; Nassini, R.; Baroni, G.; Rulli, E.; Massi, D.; Oliviero, S.; Piva, R.; Taverna, D.; Mandala, M.; Deaglio, S., PD-Li up-regulation in melanoma increases disease aggressiveness and is mediated through miR-17-5p. Oncotarget 2017, 8 (9), 15894-15911.
55. Yamazaki, T.; Sasaki, N.; Nishi, M.; Yamazaki, D.; Ikeda, A.; Okuno, Y.; Komazaki, S.; Takeshima, H., Augmentation of drug-induced cell death by ER protein BRI3BP. Biochem. Biophys. Res. Commun. 2007, 362 (4), 971-5.
56. Mendes, M.; Perez-Hernandez, D.; Vazquez, J.; Coelho, A. V.; Cunha, C., Proteomic changes in HEK-293 cells induced by hepatitis delta virus replication. J. Proteomics 2013, 89, 24-38.
57. Costales, M. G.; Aikawa, H.; Li, Y.; Childs-Disney, J. L.; Abegg, D.; Hoch, D. G.; Pradeep Velagapudi, S.; Nakai, Y.; Khan, T.; Wang, K. W.; Yildirim, I.; Adibekian, A.; Wang, E. T.; Disney, M. D., Small-molecule targeted recruitment of a nuclease to cleave an oncogenic RNA in a mouse model of metastatic cancer. Proc. Natd. Acad. Sci. U.S.A. 2020, 117 (5), 201914286.
58. Costales, M. G.; Suresh, B.; Vishnu, K.; Disney, M. D., Targeted degradation of a hypoxia-associated non-coding RNA enhances the selectivity of a small molecule interacting with RNA. Cell Chem. Biol. 2019, 26 (8), 1180-1186.e5.
59. Floyd-Smith, G.; Slattery, E.; Lengyel, P., Interferon action: RNA cleavage pattern of a (2′-5′)oligoadenylate-dependent endonuclease. Science 1981, 212 (4498), 1030-2.
60. Wreschner, D. H.; McCauley, J. W.; Skehel, J. J.; Kerr, I. M., Interferon action-sequence specificity of the ppp(A2′p)nA-dependent ribonuclease. Nature 1981, 289 (5796), 414-7.
61. Lima, W. F.; Monia, B. P.; Ecker, D. J.; Freier, S. M., Implication of RNA structure on antisense oligonucleotide hybridization kinetics. Biochemistry 1992, 31 (48), 12055-61.
62. Zaug, A.; Cech, T., The intervening sequence RNA of Tetrahymena is an enzyme. Science 1986, 231 (4737), 470-475.
63. Doudna, J. A., Structural genomics of RNA. Nat. Struct. Biol. 2000, 7 Suppl (11), 954-6.
64. Ding, Y.; Tang, Y.; Kwok, C. K.; Zhang, Y.; Bevilacqua, P. C.; Assmann, S. M., In vivo genome-wide profiling of RNA secondary structure reveals novel regulatory features. Nature 2014, 505 (7485), 696-700.

### EXPERIMENTAL SECTION REFERENCES

1. Angelbello, A. J.; Disney, M. D., Bleomycin can cleave an oncogenic noncoding RNA. Chembiochem 2018, 19 (1), 43-47.
2. Zou, W. P.; Wolchok, J. D.; Chen, L. P., PD-L1 (B7-H1) and PD-1 pathway blockade for cancer therapy: Mechanisms, response biomarkers, and combinations. Sci. Transl. Med. 2016, 8 (328), 328rv4-328rv4.
3. Boussiotis, V. A., Molecular and biochemical aspects of the PD-1 checkpoint pathway. N. Engl. J. Med. 2016, 375 (18), 1767-1778.
4. Zhang, J.; Bu, X.; Wang, H.; Zhu, Y.; Geng, Y.; Nihira, N. T.; Tan, Y.; Ci, Y.; Wu, F.; Dai, X.; Guo, J.; Huang, Y.-H.; Fan, C.; Ren, S.; Sun, Y.; Freeman, G. J.; Sicinski, P.; Wei, W., Cyclin D-CDK4 kinase destabilizes PD-L1 via cullin 3-SPOP to control cancer immune surveillance. Nature 2017, 553, 91.
5. Gotwals, P.; Cameron, S.; Cipolletta, D.; Cremasco, V.; Crystal, A.; Hewes, B.; Mueller, B.; Quaratino, S.; Sabatos-Peyton, C.; Petruzzelli, L.; Engelman, J. A.; Dranoff, G., Prospects for combining targeted and conventional cancer therapy with immunotherapy. Nat. Rev. Cancer 2017, 17, 286.
6. Rao, X.; Huang, X.; Zhou, Z.; Lin, X., An improvement of the 2\hat{ }(−delta delta CT) method for quantitative real-time polymerase chain reaction data analysis. Biostat. Bioinforma. Biomath. 2013, 3 (3), 71-85.
7. Velagapudi, S. P.; Luo, Y.; Tran, T.; Haniff, H. S.; Nakai, Y.; Fallahi, M.; Martinez, G. J.; Childs-Disney, J. L.; Disney, M. D., Defining RNA-small molecule affinity landscapes enables design of a small molecule inhibitor of an oncogenic noncoding RNA. ACS Cent. Sci. 2017, 3 (3), 205-216.
8. Velagapudi, S. P.; Cameron, M. D.; Haga, C. L.; Rosenberg, L. H.; Lafitte, M.; Duckett, D. R.; Phinney, D. G.; Disney, M. D., Design of a small molecule against an oncogenic noncoding RNA. Proc. Natl. Acad. Sci. U.S.A 2016, 113 (21), 5898-903.
9. Cox, J.; Mann, M., MaxQuant enables high peptide identification rates, individualized p.p.b.-range mass accuracies and proteome-wide protein quantification. Nat. Biotechnol. 2008, 26 (12), 1367-72.
10. Costales, M. G.; Aikawa, H.; Li, Y.; Childs-Disney, J. L.; Abegg, D.; Hoch, D. G.; Velagapudi, S. P.; Nakai, Y.; Khan, T.; Wang, K. W.; Yildirim, I.; Adibekian, A.; Wang, E. T.; Disney, M. D., Small-molecule targeted recruitment of a nuclease to cleave an oncogenic RNA in a mouse model of metastatic cancer. Proc. Natl. Acad. Sci. U.S.A. 2020, doi/10.1073/pnas.1914286117.

### SUMMARY STATEMENTS

The inventions, examples, biological assays and results described and claimed herein have may attributes and embodiments include, but not limited to, those set forth or described or referenced in this application.

All patents, publications, scientific articles, web sites and other documents and material references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, textbook or other referenced material or document.

The written description of this patent application includes all claims. All claims including all original claims are hereby incorporated by reference in their entirety into the written description portion of the specification and the right is reserved to physically incorporated into the written description or any other portion of the application any and all such claims. Thus, for example, under no circumstances may the patent be interpreted as allegedly not providing a written description for a claim on the assertion that the precise wording of the claim is not set forth in haec verba in written description portion of the patent.

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred nonlimiting embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

1. A method for targeting an RNA entity comprising contacting the RNA entity with a binding composition wherein
the RNA entity is a pre-miRNA-X selected from one or more of pre-miR-17, pre-miR-18a, pre-miR-19a, pre-miR-19b-1, pre-miR-20a, and pre-miR-92a-1 or any combination thereof, pri-miR-17-92 cluster, an oncogenic cell line containing one or more of pre-miRNA-X and/or pri-R-17-92 cluster, the cell line being TNBC breast cancer cell line, MDA-MD-231 breast cancer cell line or DU-145 prostate cancer cell line, WT 9-12 polycystic kidney cell line, an animal host having the oncogenic cell line, or a human having the oncogenic malignancy; or an animal host having polycystic kidney disease, or a human having polycystic kidney disease; or any disease in which members of the pri-miR-17-92 cluster and/or pre-RNA-X are causative or contributive; and, the binding composition is selected from one or more of Compound 1D, Compound 2, Compound 5 or Compound 7 or any combination thereof, wherein

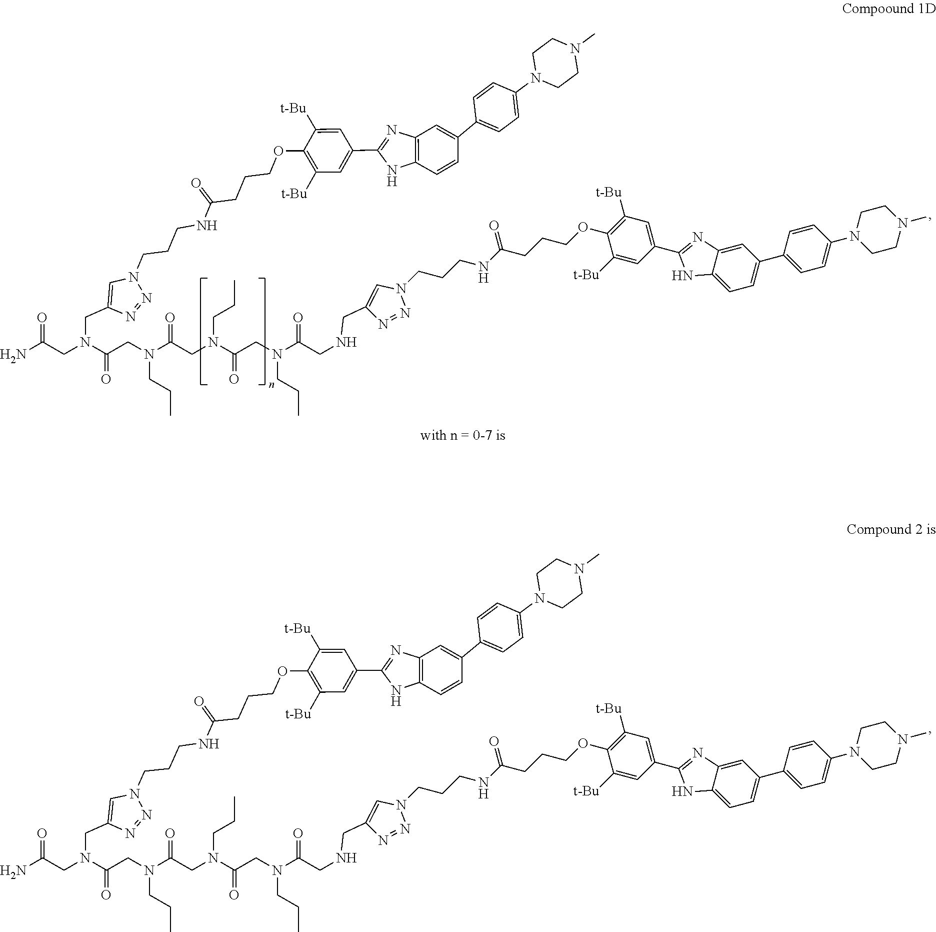

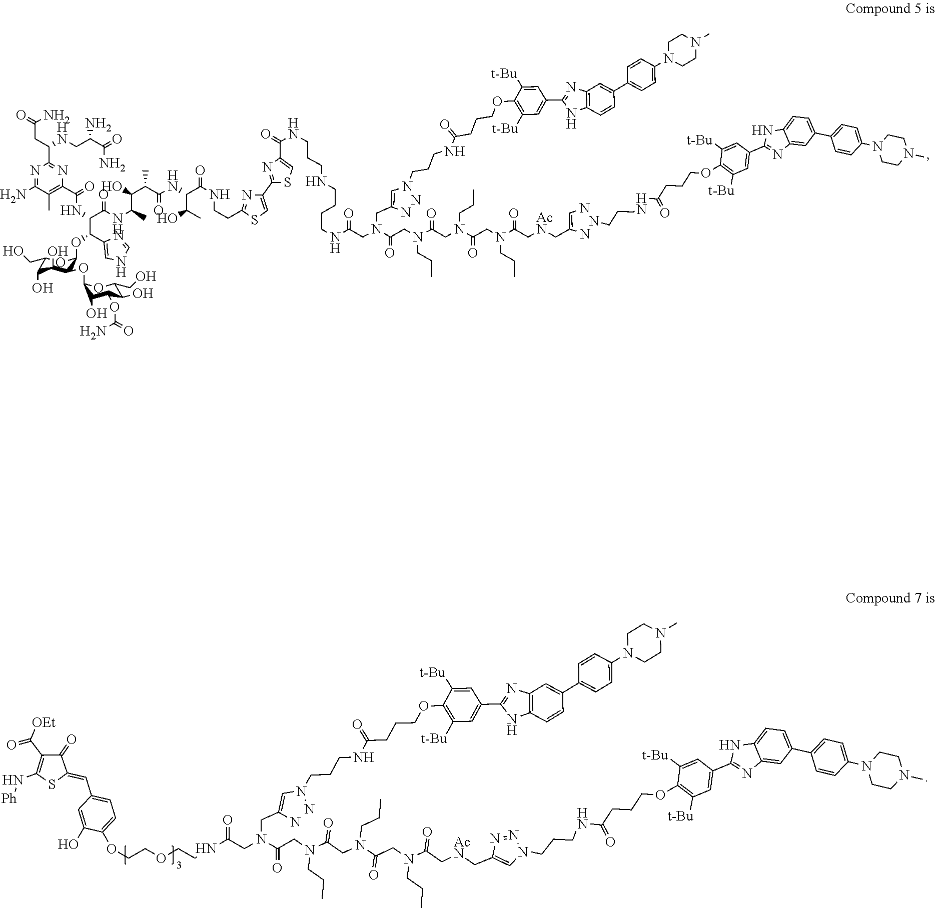

2. A method according to claim 1 for targeting at least one or more pre-miRNA-X's or targeting one or more pre-miRNA-X's embedded within the pri-miR-17-92 cluster or targeting the cluster itself comprising contacting Compound 1D wherein n is 0-7 with one or more of pre-miRNA-X's selected from pre-miR-17, pre-miR-18a, pre-miR-20a or a mixture thereof.

3. (canceled)

4. (canceled)

5. A method according to claim 2, wherein Compound 1D with n as 3 is Compound 2.

6.-9. (canceled)

10. A method according to claim 1 for targeting a pre-miRNA comprising contacting a pre-miRNA with Compound 2 wherein the pre-miRNA comprises one or more of pre-miR-17, pre-miR-18a, pre-miR-20a, a mixture thereof, or as part of pri-miR17-92.

11.-13. (canceled)

14. A method according to claim 1 wherein a TNBC breast cell cancer line is treated by contacting the cell line with Compound 2 and the cellular levels of one or more of mature miR-17, miR-18a, miR-20a or a mixture thereof are decreased.

15. (canceled)

16. A method according to claim 1 wherein an MDA-MB-231 breast cancer cell line is treated by contacting the cell line with Compound 2.

17.-21. (canceled)

22. A method according to claim 1 wherein a prostate cell cancer line is treated by contacting the cell line with Compound 2 and the cellular levels of one or more of mature miR-17, miR-18a, miR-20a or a mixture thereof are decreased.

23. (canceled)

24. A method according to claim 1 wherein a DU-145 prostate cancer cell line is treated by contacting the cell line with Compound 2.

25.-31. (canceled)

32. A method according to claim 1 for treating a polycystic kidney cell line comprising contacting the cell line with Compound 2.

33.-37. (canceled)

38. A method according to claim 1 for treating prostate and/or breast cancer cells and or polycystic kidney disease cells comprising contacting the cells with Compound 2.

39. A method according to claim 38 wherein the prostate and breast cancer cells are DU-145 prostate cancer cells and MDA-MB-231 or TNBC breast cancer cells and the polycystic kidney disease cells are WT 9-12.

40.-42. (canceled)

43. A method according to claim 1 comprising affecting degradation of the pri-miR-17-92 cluster or one or more pre-miRNAs by contacting the one or more pre-miRNAs or the pri-miRNA with Compound 5 wherein the one or more pre-miRNAs are selected from one or more of pre-miR-17, pre-miR-18a, pre-miR-20a or a mixture thereof.

44. A method according to claim 1 for treating TNBC breast cancer cells comprising contacting the TNBC cells with Compound 5.

45.-47. (canceled)

48. A method according to claim 1 for treating MDA-MB-231 breast cancer cells comprising contacting the cells with Compound 5 so as to diminish the invasive characteristic of the cells and/or to cause apoptosis of the cells.

49.-51. (canceled)

52. A method according to claim 1 for treating DU-145 prostate cancer cells comprising contacting the prostate cancer cells with Compound 5 thereby decreasing the level of the pri-miR-17-92 cluster, the pre-miRNAs therein, and the encoded mature miRNAs of the cells.

53.-55. (canceled)

56. A method according to claim 1 for treating MDA-MB-231 and/or TNBC cancer cells or polycystic kidney cells comprising contacting the cells with Compound 7.

57. A method according to claim 1 for treating DU-145 prostate cancer cells comprising contacting the cells with Compound 7.

58.-61. (canceled)

62. A composition comprising Compound 1D, 2, 4FL, 5 or 7 or any combination thereof wherein

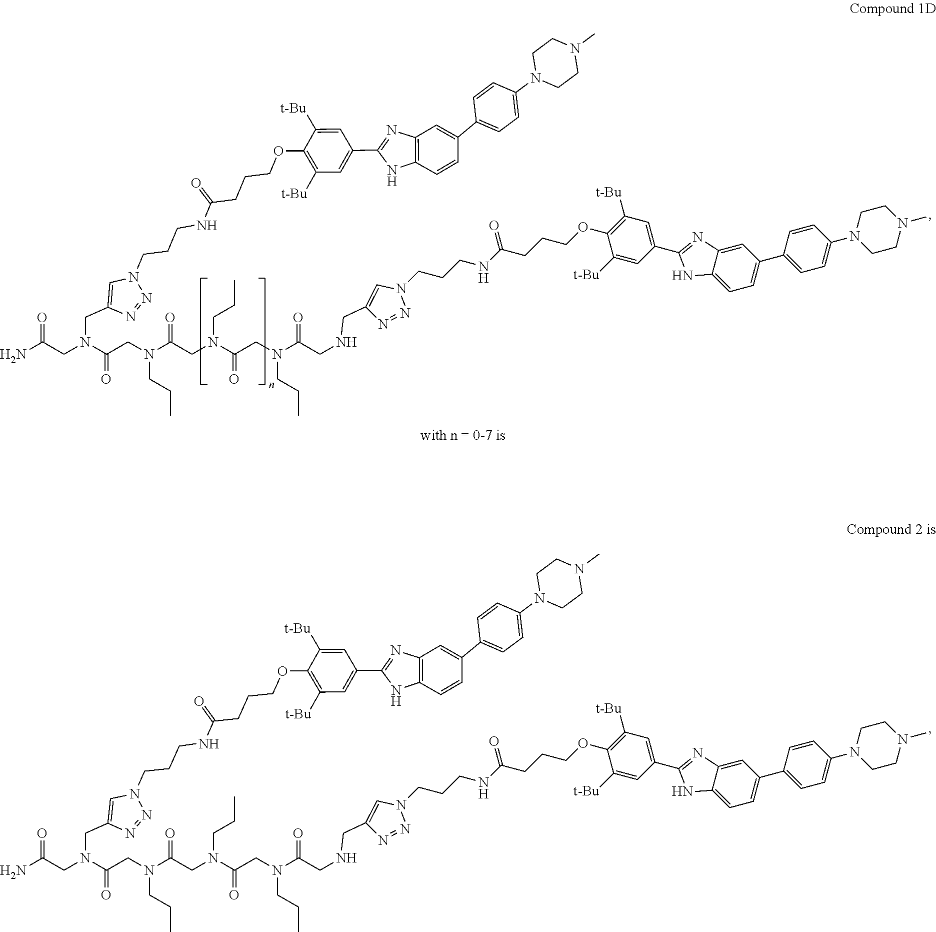

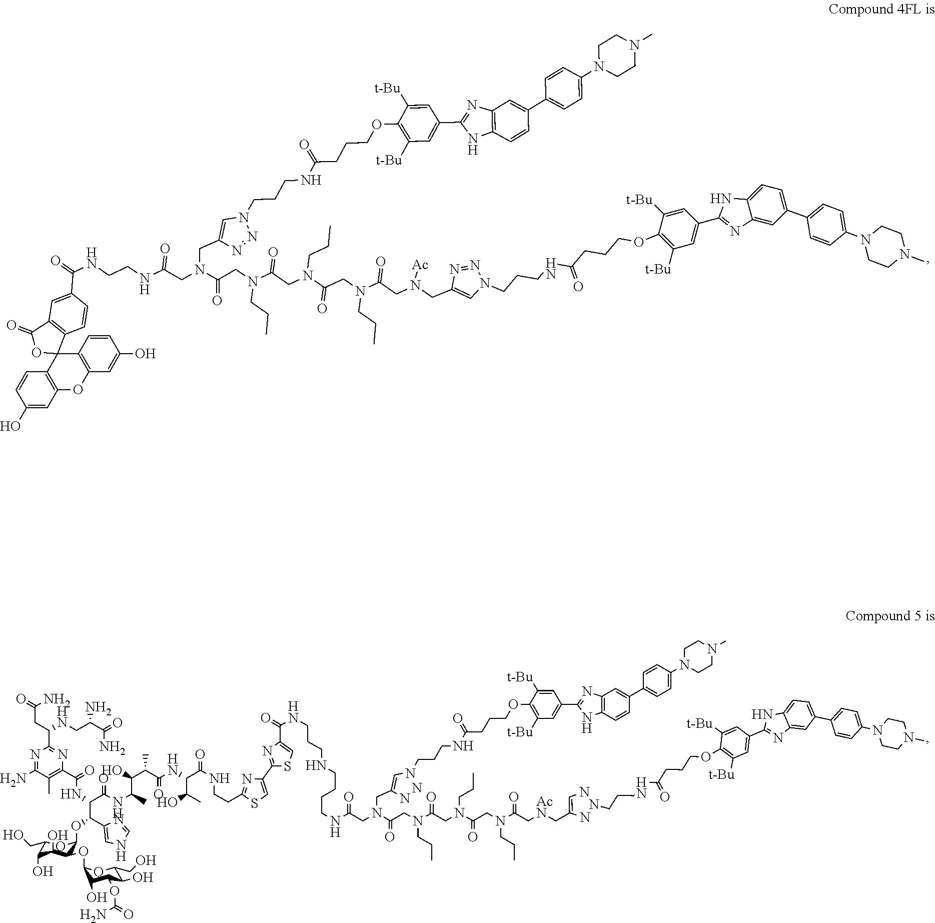

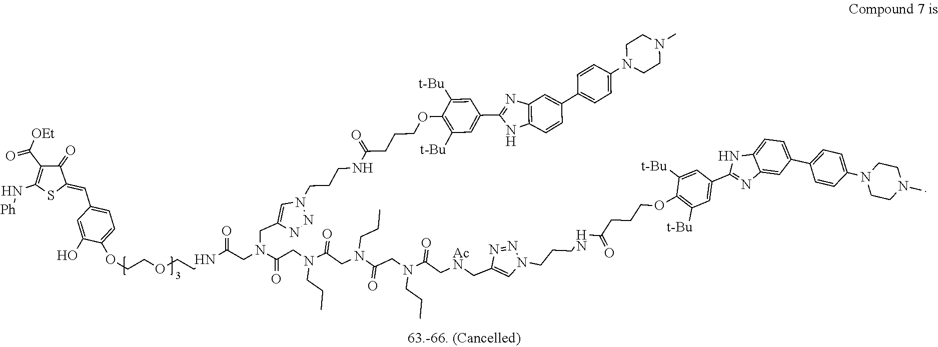

67. A pharmaceutical composition comprising a composition of claim 62 and a pharmaceutically acceptable carrier.

68. A method for treatment of prostate or breast cancer cells or polycystic kidney cells comprising contacting the cells with a pharmaceutical composition of claim 67.

69.-80. (canceled)
